(19) United States
(12) Patent Application Publication
(10) Pub. No.: US 2024/0374602 A1
(43) Pub. Date: Nov. 14, 2024

(54) METHODS FOR TREATING NEUROGENERATIVE DISEASES

(71) Applicant: PTC THERAPEUTICS, INC., South Plainfield, NJ (US)

(72) Inventors: Scott J. Barraza, Piscataway, NJ (US); Jeffrey K. Trimmer, San Carlos, CA (US); Yong Yu, Westfield, NJ (US); Xiaoyan Zhang, Belle Mead, NJ (US)

(21) Appl. No.: 18/689,680

(22) PCT Filed: Sep. 6, 2022
(86) PCT No.: PCT/US2022/075969

§ 371 Date: Mar. 6, 2024

Related U.S. Application Data
(60) Provisional application No. 63/260,944, filed on Sep. 7, 2021.

(51) Int. Cl. A61K 31/519; A61K 31/5355

(52) U.S. Cl. CPC A61K 31/519; A61K 31/5355

(57) ABSTRACT

The present description relates to methods of treating neurodegenerative diseases characterized by the accumulation of aberrant forms of the microtubule associated protein Tau (MAPT) using substituted thieno[3,2-d]pyrimidine compounds, forms, and pharmaceutical compositions thereof.

### CROSS-REFERENCE TO RELATED APPLICATIONS

This application claims the benefit of, and priority to U.S. Provisional Patent Application No. 63/260,944 filed on Sep. 7, 2021, the contents of which are herein incorporated by reference in its entirety for all purposes.

### TECHNICAL FIELD

The present description relates to methods of treating neurodegenerative diseases characterized by the accumulation of aberrant forms of the microtubule associated protein Tau (MAPT) using substituted thieno[3,2-d]pyrimidine compounds, forms, and pharmaceutical compositions thereof.

### BACKGROUND

Tauopathies are a group of neurodegenerative diseases characterized by the accumulation and aggregation of aberrant forms of the microtubule associated protein Tau (MAPT) leading to the formation of neurofibrillary tangles (NFT) and paired helical filaments (PHF) in neurons and glia of the affected brain regions. Accumulation and aggregation of tau is the main pathological hallmark of more than 18 irreversible neurodegenerative diseases, collectively referred to as tauopathies. These diseases include frontotemporal dementia (FTD), progressive supranuclear palsy (PSP) and Alzheimer's disease (AD), and can be either sporadic or inherited when caused by mutations in the MAPT gene 1. It is estimated that Tauopathies account for 10-20% of all dementia cases; affecting ˜55,000 people in the United States. Currently, there are no effective disease-modifying therapies and few experimental drugs focused on Tau are undergoing clinical trials.

The tau protein is encoded by the MAPT gene located on chromosome 17q21 and is required for the stabilization and assembly of microtubules. Microtubules are important for axonal transport and for maintaining the structural integrity of the cell. In the adult brain, tau is located within neurons, predominantly within axons. Tau is also found in oligodendrocytes and astrocytes in which its function is similar to its function in neurons. The binding of tau to the microtubule can induce a conformational change in the protein. In its normal form, tau is unfolded and phosphorylated. In the brains of patients with primary tauopathies, tau is hyperphosphorylated and with a folded β-pleated sheet conformation resulting in aggregation.

The binding of tau to microtubules is regulated by the ratio of
phosphorylation/dephosphorylation. Tau hyperphosphorylation results in a loss of microtubule interaction, leading to microtubule dysfunction and impaired axonal transport as well as to tau fibrillization. Recently, it has been suggested that the proportion of tau sequences that are phosphorylated, as opposed to the number of phosphorylated epitopes on each tau sequence, predicts the degree of aggregation and formation of NFTs.

There are 6 isoforms of tau expressed in the adult brain. These 6 isoforms are derived from the alternative splicing of 3 N-terminal exons in the tau gene: exon 2, exon 3, and exon 10. Three of the 6 isoforms are due to the splicing in of exon 10, whereas the other 3 isoforms are a result of the splicing out of exon 10. The splicing in of exon 10 results in isoforms with 4 repeated microtubule-binding domains (4R tau), whereas the splicing out of exon 10 results in isoforms with 3 repeated microtubule-binding domains (3R tau). This is important because although the healthy human brain consists of equal amounts of tau with 3 and 4 repeated microtubule-binding domains, some primary tauopathies are characterized by a predominance of isoforms with 4 repeated microtubule-binding domains (4R tauopathies), some by a predominance of isoforms with 3 repeated microtubule-binding domains (3R tauopathies), and some by an approximately equal mix of isoforms with 3 and 4 repeated microtubule-binding domains (3R+4R tauopathies). Tau neurofibrillary tangles in disease have different isoforms' composition, suggesting that splicing is an important target potential therapeutics.

MAPT mutations that affect splicing of exons 2 and 3 are very rare, while pathogenic mutations in the exon 10 and exon10-intron10 boundary regions are more common, representing ˜27% of all known Tau mutations. Most of the pathogenic mutations result in mis-splicing which in most cases increases the inclusion of exon 10 and 4R-Tau expression.

The present description relates to the use of a compound of Formula (I) or a form or composition thereof for treating tauopathies. These sets of compounds induce exon 10 skipping in the MAPT pre-mRNA during the splicing process. Exon 10 skipping of MAPT mRNA changes the open reading frame (ORF) and creates premature termination codons (PTCs) in the MAPT4R exon 10-skipped mRNA (ΔE4 mRNA). It has been shown that such exon skipping splicing events could serve to reduce gene expression by creating mRNAs with premature termination codons, thus signaling the mRNAs to be degraded rather than translated into proteins. Similarly, MAPT ΔE4 mRNA produced in the presence of these compounds will undergo mRNA degradation resulting in decreased levels of MAPT 4R mRNA, resulting in MAPT4R protein lowering.

International Publication No. WO2016/115434 discloses kinetin derivatives useful for improving mRNA splicing in a cell, and in particular for improving mRNA splicing in genes having at least one exon ending in the nucleotide sequence CAA such as the TKBKAP gene. The compounds disclosed may be used for treating diseases of the central nervous system such as familial dysautonomia.

To date, there are no disease-modifying therapies available for tauopathies, and there exists a need for improved methods and compositions for treating tauopathies and the symptoms associated therewith. International Publication No. WO2020/167624 discloses substituted pyrrolo[2,3-d]pyrimidine compounds useful for therapeutically targeting pre-mRNA splicing mechanisms in the IKBKAP gene and for the treatment of familial dysautonomia. Neither application discloses compounds that induce exon 10 skipping in MAPT pre-mRNA splicing. In addition, neither application discloses compounds that result in MAPT protein lowering, in particular MAPT protein lowering due to mRNA degradation of MAPT 4R mRNA produced in the presence of the compounds. Furthermore, neither application discloses compounds that are useful for treating tauopathies.

The compounds described herein represent potential MAPT pre-mRNA splicing compounds that could be used as a disease-modifying treatment for a variety of tauopathies.

All other documents referred to herein are incorporated by reference into the present application as though fully set forth herein.

### SUMMARY

The present description relates to a method or use of a compound for treating neurodegenerative diseases characterized by the accumulation of aberrant forms of the microtubule associated protein Tau (MAPT) in a subject in need thereof comprising administering to the subject an effective amount of a compound of Formula (I):

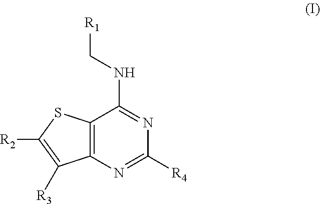

or a form thereof, wherein R₁, R₂, R₃, and R₄ are defined herein.

### DETAILED DESCRIPTION

An aspect of the present description relates to method or use of a compound for treating neurodegenerative diseases characterized by the accumulation of aberrant forms of the microtubule associated protein Tau (MAPT) in a subject in need thereof comprising administering to the subject an effective amount of a compound of Formula (I):

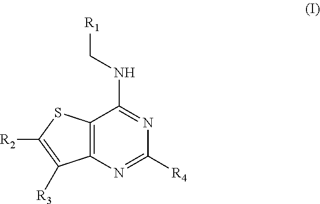

or a form thereof, wherein:
R₁ is selected from the group consisting of phenyl and heteroaryl,
wherein heteroaryl is a 5-8 membered monocyclic or bicyclic aromatic carbon atom ring structure radical containing 1-3 heteroatoms selected from N, O, and S, and
wherein phenyl or heteroaryl is optionally substituted with one, two, three, or four, independently selected R₁ₐ substituents; R₁ₐ is independently selected from the group consisting of cyano, halo, hydroxy,
C₁₋₆alkyl, halo-C₁₋₆alkyl, deutero-C₁₋₆alkyl, and C₁₋₆alkoxy;
R₂ is selected from the group consisting of hydrogen, C₁₋₆alkyl, C₂₋₆alkenyl, C₂₋₆alkynyl, C₃₋₁₀cycloalkyl, phenyl, heterocyclyl, and heteroaryl,
wherein heterocyclyl is a 3-7 membered monocyclic carbon atom ring structure radical containing 1-3 heteroatoms selected from N, O, and S,
wherein heteroaryl is a 5-8 membered monocyclic or bicyclic aromatic carbon atom ring structure radical containing 1-3 heteroatoms selected from N, O, and S,
wherein C₁₋₆alkyl, C₂₋₆alkenyl, and C₂₋₆alkynyl optionally contain a chiral carbon having an (R) or (S) configuration, and
wherein C₁₋₆alkyl, C₂₋₆alkenyl, C₂₋₆alkynyl, C₃₋₁₀cycloalkyl, phenyl, heterocyclyl, or heteroaryl are optionally substituted with one, two, three, or four independently selected R₂ₐ substituents;
R₂ₐ is independently selected from the group consisting of cyano, halo, hydroxy, oxo, C₁₋₆alkyl, halo-C₁₋₆alkyl, deutero-C₁₋₆alkyl, C₂₋₆alkenyl, C₂₋₆alkynyl, C₁₋₆alkoxy, halo-C₁₋₆alkoxy, carboxyl, amino, C₁₋₆alkyl-amino, halo-C₁₋₆alkyl-amino, deutero-C₁₋₆alkyl-amino, (C₁₋₆alkyl)₂-amino, C₃₋₁₀cycloalkyl-amino, phenyl-amino, heterocyclyl-amino, heteroaryl-amino, C₁₋₆alkyl-thio, C₁₋₆alkyl-sulfonyl, C₃₋₁₀cycloalkyl, phenyl, heterocyclyl, and heteroaryl,
wherein heterocyclyl is a 3-7 membered monocyclic carbon atom ring structure radical containing 1-3 heteroatoms selected from N, O, and S,
wherein heteroaryl is a 5-8 membered monocyclic or bicyclic aromatic carbon atom ring structure radical containing 1-3 heteroatoms selected from N, O, and S, and
wherein each instance of C₃₋₁₀cycloalkyl, phenyl, heterocyclyl or heteroaryl is optionally substituted with one, two, three or four independently selected R_{2a′} substituents;
R_{2a′} is independently selected from the group consisting of cyano, halo, hydroxy, oxo, C₁₋₆alkyl, halo-C₁₋₆alkyl, deutero-C₁₋₆alkyl, and C₁₋₆alkoxy;
R₃ is selected from the group consisting of hydrogen, cyano, halo, hydroxy, C₁₋₆alkyl, halo-C₁₋₆alkyl, C₁₋₆alkoxy, amino, C₁₋₆alkyl-amino, (C₁₋₆alkyl)₂-amino, C₃₋₁₀cycloalkyl, phenyl, heterocyclyl, and heteroaryl,
wherein heterocyclyl is a 3-7 membered monocyclic carbon atom ring structure radical containing 1-3 heteroatoms selected from N, O, and S, and
wherein heteroaryl is a 5-8 membered monocyclic or bicyclic aromatic carbon atom ring structure radical containing 1-3 heteroatoms selected from N, O, and S, and
wherein C₁₋₆alkyl, C₃₋₁₀cycloalkyl, phenyl, heterocyclyl, or heteroaryl is optionally substituted with one, two, three, or four independently selected R₃ₐ substituents;
R₃ₐ is independently selected from the group consisting of cyano, halo, hydroxy, C₁₋₆alkyl, halo-C₁₋₆alkyl, and C₁₋₆alkoxy;
R₄ is selected from the group consisting of hydrogen, cyano, halo, hydroxy, C₁₋₆alkyl, halo-C₁₋₆alkyl, C₁₋₆alkoxy, carbamoyl, C₃₋₁₀cycloalkyl, phenyl, and heterocyclyl, and
wherein heterocyclyl is a 3-7 membered monocyclic carbon atom ring structure radical containing 1-3 heteroatoms selected from N, O, and S; and
wherein the form of the compound is selected from the group consisting of a salt, hydrate, solvate, and tautomer form thereof.

One aspect of the method or use includes a compound of Formula (I), wherein R₁ is selected from the group consisting of phenyl and heteroaryl,
wherein heteroaryl is a 5-8 membered monocyclic or bicyclic aromatic carbon atom ring structure radical containing 1-3 heteroatoms selected from N, O, and S, and
wherein phenyl or heteroaryl are optionally substituted with one, two, three, or four, independently selected R₁ₐ substituents.

Another aspect of the method or use includes a compound of Formula (I), wherein R₁ is selected from the group consisting of phenyl and heteroaryl,
wherein heteroaryl is a 5-8 membered monocyclic or bicyclic aromatic carbon atom ring structure radical containing 1-3 heteroatoms selected from N, O, and S, and
wherein phenyl or heteroaryl is optionally substituted with one or two independently selected R₁ₐ substituents.

Another aspect of the method or use includes a compound of Formula (I), wherein R₁ is phenyl, and wherein phenyl is optionally substituted with one, two, three, or four independently selected R₁ₐ substituents.

Another aspect of the method or use includes a compound of Formula (I), wherein R₁ is phenyl, and wherein phenyl is substituted with one R₁ₐ substituent.

Another aspect of the method or use includes a compound of Formula (I), wherein R₁ is heteroaryl,
wherein heteroaryl is a 5-8 membered monocyclic or bicyclic aromatic carbon atom ring structure radical containing 1-3 heteroatoms selected from N, O, and S, and
wherein heteroaryl is optionally substituted with one, two, three, or four independently selected R₁ₐ substituents.

Another aspect of the method or use includes a compound of Formula (I), wherein R₁ is heteroaryl,
wherein heteroaryl is a 5-8 membered monocyclic or bicyclic aromatic carbon atom ring structure radical containing 1-3 heteroatoms selected from N, O, and S, and
wherein heteroaryl is optionally substituted with one or two independently selected R₁ₐ substituents.

Another aspect of the method or use includes a compound of Formula (I), wherein R₁ is heteroaryl selected from the group consisting of furanyl, thiophenyl, 1H-pyrrolyl, 1H-pyrazolyl, 1H-imidazolyl, 1H-1,2,3-triazolyl, 2H-1,2,3-triazolyl, 1H-tetrazolyl, 1,2-thiazolyl, 1,3-thiazolyl, 1,2-oxazolyl, 1,3-oxazolyl, 1,2,4-oxadiazolyl, 1,3,4-oxadiazolyl, 1,2,3-thiadiazolyl, pyridinyl, pyrimidinyl, pyrazinyl, pyridazinyl, benzofuranyl, and quinolinyl, and
wherein heteroaryl is optionally substituted with one, two, three, or four independently selected R₁ₐ substituents.

Another aspect of the method or use includes a compound of Formula (I), wherein R₁ is heteroaryl selected from the group consisting of furanyl, thiophenyl, 1H-pyrrolyl, 1H-pyrazolyl, 1H-imidazolyl, 1H-1,2,3-triazolyl, 2H-1,2,3-triazolyl, 1H-tetrazolyl, 1,2-thiazolyl, 1,3-thiazolyl, 1,2-oxazolyl, 1,3-oxazolyl, 1,2,4-oxadiazolyl, 1,3,4-oxadiazolyl, 1,2,3-thiadiazolyl, pyridinyl, pyrimidinyl, pyrazinyl, pyridazinyl, benzofuranyl, and quinolinyl, and
wherein heteroaryl is optionally substituted with one or two independently selected R₁ₐ substituents.

Another aspect of the method or use includes a compound of Formula (I), wherein R₁ is heteroaryl selected from the group consisting of furanyl, thiophenyl, 1H-pyrrolyl, 1H-pyrazolyl, 1H-imidazolyl, 1H-1,2,3-triazolyl, 2H-1,2,3-triazolyl, 1H-tetrazolyl, 1,2-thiazolyl, 1,3-thiazolyl, 1,2-oxazolyl, 1,3-oxazolyl, 1,2,4-oxadiazolyl, 1,3,4-oxadiazolyl, 1,2,3-thiadiazolyl, pyridinyl, pyrimidinyl, pyrazinyl, pyridazinyl, benzofuranyl, and quinolinyl, and
wherein heteroaryl is optionally substituted with one R₁ₐ substituent.

Another aspect of the method or use includes a compound of Formula (I), wherein R₁ is heteroaryl selected from the group consisting of furanyl, thiophenyl, 1H-pyrrolyl, 1H-pyrazolyl, 1H-imidazolyl, 1H-1,2,3-triazolyl, 2H-1,2,3-triazolyl, 1H-tetrazolyl, 1,2-thiazolyl, 1,3-thiazolyl, 1,2-oxazolyl, 1,3-oxazolyl, 1,2,4-oxadiazolyl, 1,3,4-oxadiazolyl, 1,2,3-thiadiazolyl, pyridinyl, pyrimidinyl, pyrazinyl, pyridazinyl, benzofuranyl, and quinolinyl, and
wherein heteroaryl is optionally substituted with two independently selected R₁ₐ substituents.

Another aspect of the method or use includes a compound of Formula (I), wherein R₁ is heteroaryl selected from the group consisting of furanyl, thiophenyl, 1H-pyrrolyl, 1H-pyrazolyl, 1H-imidazolyl, 2H-1,2,3-triazolyl, 1H-tetrazolyl, 1,2-thiazolyl, 1,3-thiazolyl, 1,2-oxazolyl, 1,3-oxazolyl, 1,2-thiazolyl, 1,3-thiazolyl, 1,2-oxazolyl, 1,3-oxazolyl, pyridinyl, pyrimidinyl, and pyrazinyl, and
wherein heteroaryl is optionally substituted with one, two, three, or four independently selected R₁ₐ substituents.

Another aspect of the method or use includes a compound of Formula (I), wherein R₁ is heteroaryl selected from the group consisting of furanyl, thiophenyl, 1H-pyrrolyl, 1H-pyrazolyl, 1H-imidazolyl, 2H-1,2,3-triazolyl, 1H-tetrazolyl, 1,2-thiazolyl, 1,3-thiazolyl, 1,2-oxazolyl, 1,3-oxazolyl, 1,2-thiazolyl, 1,3-thiazolyl, 1,2-oxazolyl, 1,3-oxazolyl, pyridinyl, pyrimidinyl, and pyrazinyl, and
wherein heteroaryl is optionally substituted with one or two independently selected R₁ₐ substituents.

Another aspect of the method or use includes a compound of Formula (I), wherein R₁ is heteroaryl selected from the group consisting of furanyl, thiophenyl, 1H-pyrrolyl, 1H-pyrazolyl, 1H-imidazolyl, 2H-1,2,3-triazolyl, 1H-tetrazolyl, 1,2-thiazolyl, 1,3-thiazolyl, 1,2-oxazolyl, 1,3-oxazolyl, 1,2-thiazolyl, 1,3-thiazolyl, 1,2-oxazolyl, 1,3-oxazolyl, pyridinyl, pyrimidinyl, and pyrazinyl, and
wherein heteroaryl is optionally substituted with one R₁ₐ substituents.

Another aspect of the method or use includes a compound of Formula (I), wherein R₁ is heteroaryl selected from the group consisting of furanyl, thiophenyl, 1H-pyrrolyl, 1H-pyrazolyl, 1H-imidazolyl, 2H-1,2,3-triazolyl, 1H-tetrazolyl, 1,2-thiazolyl, 1,3-thiazolyl, 1,2-oxazolyl, 1,3-oxazolyl, 1,2-thiazolyl, 1,3-thiazolyl, 1,2-oxazolyl, 1,3-oxazolyl, pyridinyl, pyrimidinyl, and pyrazinyl, and
wherein heteroaryl is optionally substituted with two independently selected R₁ₐ substituents.

Another aspect of the method or use includes a compound of Formula (I), wherein R₁ is heteroaryl selected from the group consisting of furan-2-yl, furan-3-yl, thiophen-2-yl, thiophen-3-yl, 1H-pyrrol-2-yl, 1H-pyrrol-3-yl, 1H-pyrazol-1-yl, 1H-pyrazol-3-yl, 1H-pyrazol-4-yl, 1H-pyrazol-5-yl, 1H-imidazol-1-yl, 1H-imidazol-2-yl, 1H-imidazol-4-yl, 1H-imidazol-5-yl, 1H-1,2,3-triazol-1-yl, 1H-1,2,3-triazol-4-yl, 2H-1,2,3-triazol-2-yl, 2H-1,2,3-triazol-4-yl, 1H-tetrazol-1-yl, 1H-tetrazol-5-yl, 1,2-thiazol-3-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2-oxazol-4-yl, 1,2-oxazol-5-yl, 1,3-oxazol-2-yl, 1,3-oxazol-4-yl, 1,3-oxazol-5-yl, 1,2,4-oxadiazol-3-yl, 1,3,4-oxadiazol-2-yl, tetrazol-5-yl, 1,2,3-triazol-4-yl, 1,2,3-triazol-5-yl, 1,2,3-thiadiazol-4-yl, 1,2,3-thiadiazol-5-yl, pyridin-2-yl, pyridin-3-yl, pyridin-4-yl, pyrimidin-2-yl, pyrimidin-4-yl, pyrazin-2-yl, pyridazin-3-yl, pyridazin-4-yl, benzofuran-2-yl, benzofuran-5-yl, and quinoline-4-yl, and
wherein heteroaryl is optionally substituted with one, two, three, or four independently selected R₁ₐ substituents.

Another aspect of the method or use includes a compound of Formula (I), wherein R₁ is heteroaryl selected from the group consisting of furan-2-yl, furan-3-yl, thiophen-2-yl, thiophen-3-yl, 1H-pyrrol-2-yl, 1H-pyrrol-3-yl, 1H-pyrazol-1-yl, 1H-pyrazol-3-yl, 1H-pyrazol-4-yl, 1H-pyrazol-5-yl, 1H-imidazol-1-yl, 1H-imidazol-2-yl, 1H-imidazol-4-yl, 1H-imidazol-5-yl, 1H-1,2,3-triazol-1-yl, 1H-1,2,3-triazol-4-yl, 2H-1,2,3-triazol-2-yl, 2H-1,2,3-triazol-4-yl, 1H-tetrazol-1-yl, 1H-tetrazol-5-yl, 1,2-thiazol-3-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2-oxazol-4-yl, 1,2-oxazol-5-yl, 1,3-oxazol-2-yl, 1,3-oxazol-4-yl, 1,3-oxazol-5-yl, 1,2,4-oxadiazol-3-yl, 1,3,4-oxadiazol-2-yl, tetrazol-5-yl, 1,2,3-triazol-4-yl, 1,2,3-triazol-5-yl, 1,2,3-thiadiazol-4-yl, 1,2,3-thiadiazol-5-yl, pyridin-2-yl, pyridin-3-yl, pyridin-4-yl, pyrimidin-2-yl, pyrimidin-4-yl, pyrazin-2-yl, pyridazin-3-yl, pyridazin-4-yl, benzofuran-2-yl, benzofuran-5-yl, and quinoline-4-yl, and
wherein heteroaryl is optionally substituted with one or two independently selected R₁ₐ substituents.

Another aspect of the method or use includes a compound of Formula (I), wherein R₁ is heteroaryl selected from the group consisting of furan-2-yl, furan-3-yl, thiophen-2-yl, thiophen-3-yl, 1H-pyrrol-2-yl, 1H-pyrrol-3-yl, 1H-pyrazol-1-yl, 1H-pyrazol-3-yl, 1H-pyrazol-4-yl, 1H-pyrazol-5-yl, 1H-imidazol-1-yl, 1H-imidazol-2-yl, 1H-imidazol-4-yl, 1H-imidazol-5-yl, 1H-1,2,3-triazol-1-yl, 1H-1,2,3-triazol-4-yl, 2H-1,2,3-triazol-2-yl, 2H-1,2,3-triazol-4-yl, 1H-tetrazol-1-yl, 1H-tetrazol-5-yl, 1,2-thiazol-3-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2-oxazol-4-yl, 1,2-oxazol-5-yl, 1,3-oxazol-2-yl, 1,3-oxazol-4-yl, 1,3-oxazol-5-yl, 1,2,4-oxadiazol-3-yl, 1,3,4-oxadiazol-2-yl, tetrazol-5-yl, 1,2,3-triazol-4-yl, 1,2,3-triazol-5-yl, 1,2,3-thiadiazol-4-yl, 1,2,3-thiadiazol-5-yl, pyridin-2-yl, pyridin-3-yl, pyridin-4-yl, pyrimidin-2-yl, pyrimidin-4-yl, pyrazin-2-yl, pyridazin-3-yl, pyridazin-4-yl, benzofuran-2-yl, benzofuran-5-yl, and quinoline-4-yl, and
wherein heteroaryl is optionally substituted with one R₁ₐ substituent.

Another aspect of the method or use includes a compound of Formula (I), wherein R₁ is heteroaryl selected from the group consisting of furan-2-yl, furan-3-yl, thiophen-2-yl, thiophen-3-yl, 1H-pyrrol-2-yl, 1H-pyrrol-3-yl, 1H-pyrazol-1-yl, 1H-pyrazol-3-yl, 1H-pyrazol-4-yl, 1H-pyrazol-5-yl, 1H-imidazol-1-yl, 1H-imidazol-2-yl, 1H-imidazol-4-yl, 1H-imidazol-5-yl, 1H-1,2,3-triazol-1-yl, 1H-1,2,3-triazol-4-yl, 2H-1,2,3-triazol-2-yl, 2H-1,2,3-triazol-4-yl, 1H-tetrazol-1-yl, 1H-tetrazol-5-yl, 1,2-thiazol-3-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2-oxazol-4-yl, 1,2-oxazol-5-yl, 1,3-oxazol-2-yl, 1,3-oxazol-4-yl, 1,3-oxazol-5-yl, 1,2,4-oxadiazol-3-yl, 1,3,4-oxadiazol-2-yl, tetrazol-5-yl, 1,2,3-triazol-4-yl, 1,2,3-triazol-5-yl, 1,2,3-thiadiazol-4-yl, 1,2,3-thiadiazol-5-yl, pyridin-2-yl, pyridin-3-yl, pyridin-4-yl, pyrimidin-2-yl, pyrimidin-4-yl, pyrazin-2-yl, pyridazin-3-yl, pyridazin-4-yl, benzofuran-2-yl, benzofuran-5-yl, and quinoline-4-yl, and
wherein heteroaryl is optionally substituted with two independently selected R₁ₐ substituents.

Another aspect of the method or use includes a compound of Formula (I), wherein R₁ is heteroaryl selected from the group consisting of furan-2-yl, furan-3-yl, thiophen-2-yl, thiophen-3-yl, 1H-pyrrol-2-yl, 1H-pyrrol-3-yl, 1H-pyrazol-3-yl, 1H-pyrazol-4-yl, 1H-pyrazol-5-yl, 1H-imidazol-2-yl, 1H-imidazol-4-yl, 1H-imidazol-5-yl, 2H-1,2,3-triazol-4-yl, 1H-tetrazol-5-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2-oxazol-4-yl, 1,2-oxazol-5-yl, 1,3-oxazol-2-yl, 1,3-oxazol-4-yl, 1,3-oxazol-5-yl, pyridin-2-yl, pyridin-3-yl, pyridin-4-yl, pyrimidin-2-yl, pyrimidin-4-yl, and pyrazin-2-yl, and
wherein heteroaryl is optionally substituted with one, two, three, or four independently selected R₁ₐ substituents.

Another aspect of the method or use includes a compound of Formula (I), wherein R₁ is heteroaryl selected from the group consisting of furan-2-yl, furan-3-yl, thiophen-2-yl, thiophen-3-yl, 1H-pyrrol-2-yl, 1H-pyrrol-3-yl, 1H-pyrazol-3-yl, 1H-pyrazol-4-yl, 1H-pyrazol-5-yl, 1H-imidazol-2-yl, 1H-imidazol-4-yl, 1H-imidazol-5-yl, 2H-1,2,3-triazol-4-yl, 1H-tetrazol-5-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2-oxazol-4-yl, 1,2-oxazol-5-yl, 1,3-oxazol-2-yl, 1,3-oxazol-4-yl, 1,3-oxazol-5-yl, pyridin-2-yl, pyridin-3-yl, pyridin-4-yl, pyrimidin-2-yl, pyrimidin-4-yl, and pyrazin-2-yl, and
wherein heteroaryl is optionally substituted with one or two independently selected R₁ₐ substituents.

Another aspect of the method or use includes a compound of Formula (I), wherein R₁ is heteroaryl selected from the group consisting of furan-2-yl, furan-3-yl, thiophen-2-yl, thiophen-3-yl, 1H-pyrrol-2-yl, 1H-pyrrol-3-yl, 1H-pyrazol-3-yl, 1H-pyrazol-4-yl, 1H-pyrazol-5-yl, 1H-imidazol-2-yl, 1H-imidazol-4-yl, 1H-imidazol-5-yl, 2H-1,2,3-triazol-4-yl, 1H-tetrazol-5-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2-oxazol-4-yl, 1,2-oxazol-5-yl, 1,3-oxazol-2-yl, 1,3-oxazol-4-yl, 1,3-oxazol-5-yl, pyridin-2-yl, pyridin-3-yl, pyridin-4-yl, pyrimidin-2-yl, pyrimidin-4-yl, and pyrazin-2-yl, and
wherein heteroaryl is optionally substituted with one R₁ₐ substituent.

Another aspect of the method or use includes a compound of Formula (I), wherein R₁ is heteroaryl selected from the group consisting of furan-2-yl, furan-3-yl, thiophen-2-yl, thiophen-3-yl, 1H-pyrrol-2-yl, 1H-pyrrol-3-yl, 1H-pyrazol-3-yl, 1H-pyrazol-4-yl, 1H-pyrazol-5-yl, 1H-imidazol-2-yl, 1H-imidazol-4-yl, 1H-imidazol-5-yl, 2H-1,2,3-triazol-4-yl, 1H-tetrazol-5-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2-oxazol-4-yl, 1,2-oxazol-5-yl, 1,3-oxazol-2-yl, 1,3-oxazol-4-yl, 1,3-oxazol-5-yl, pyridin-2-yl, pyridin-3-yl, pyridin-4-yl, pyrimidin-2-yl, pyrimidin-4-yl, and pyrazin-2-yl, and
wherein heteroaryl is optionally substituted with two independently selected R₁ₐ substituents.

One aspect of the method or use includes a compound of Formula (I), wherein R₁ₐ is independently selected from the group consisting of cyano, halo, hydroxy, C₁₋₆alkyl, halo-C₁₋₆alkyl, deutero-C₁₋₆alkyl, and C₁₋₆alkoxy.

Another aspect of the method or use includes a compound of Formula (I), wherein R₁ₐ is independently selected from the group consisting of halo and C₁₋₆alkyl.

Another aspect of the method or use includes a compound of Formula (I), wherein R₁ₐ is halo, wherein halo is selected from the group consisting of fluoro, chloro, bromo, and iodo.

Another aspect of the method or use includes a compound of Formula (I), wherein R₁ₐ is fluoro.

Another aspect of the method or use includes a compound of Formula (I), wherein R₁ₐ is C₁₋₆alkyl selected from the group consisting of methyl, ethyl, propyl, butyl, pentyl, and hexyl.

Another aspect of the method or use includes a compound of Formula (I), wherein R₁ₐ is methyl.

One aspect of the method or use includes a compound of Formula (I), wherein R₂ is selected from the group consisting of hydrogen, C₁₋₆alkyl, C₂₋₆alkenyl, C₂₋₆alkynyl, C₃₋₁₀cycloalkyl, phenyl, heterocyclyl, and heteroaryl,
wherein heterocyclyl is a 3-7 membered monocyclic carbon atom ring structure radical containing 1-3 heteroatoms selected from N, O, and S,
wherein heteroaryl is a 5-8 membered monocyclic or bicyclic aromatic carbon atom ring structure radical containing 1-3 heteroatoms selected from N, O, and S,
wherein C₁₋₆alkyl, C₂₋₆alkenyl, and C₂₋₆alkynyl optionally contain a chiral carbon having an (R) or (S) configuration, and
wherein C₁₋₆alkyl, C₂₋₆alkenyl, C₂₋₆alkynyl, C₃₋₁₀cycloalkyl, phenyl, heterocyclyl, and heteroaryl are optionally substituted with one, two, three, or four independently selected R₂ₐ substituents.

Another aspect of the method or use includes a compound of Formula (I), wherein R₂ is hydrogen.

Another aspect of the method or use includes a compound of Formula (I), wherein R₂ is C₁₋₆alkyl,
wherein C₁₋₆alkyl optionally contains a chiral carbon having an (R) or (S) configuration, and
wherein C₁₋₆alkyl is optionally substituted with one, two, three, or four, independently selected R₂ₐ substituents.

Another aspect of the method or use includes a compound of Formula (I), wherein R₂ is C₁₋₆alkyl,
wherein C₁₋₆alkyl contains a chiral carbon having an (R) configuration, and
wherein C₁₋₆alkyl is optionally substituted with one, two, three, or four, independently selected R₂ₐ substituents.

Another aspect of the method or use includes a compound of Formula (I), wherein R₂ is C₁₋₆alkyl,
wherein C₁₋₆alkyl contains a chiral carbon having an (S) configuration, and
wherein C₁₋₆alkyl is optionally substituted with one, two, three, or four, independently selected R₂ₐ substituents.

Another aspect of the method or use includes a compound of Formula (I), wherein R₂ is C₁₋₆alkyl selected from the group consisting of methyl, ethyl, propyl, butyl, pentyl, and hexyl,
wherein C₁₋₆alkyl optionally contains a chiral carbon having an (R) or (S) configuration, and
wherein C₁₋₆alkyl is optionally substituted with one, two, three, or four, independently selected R₂ₐ substituents.

Another aspect of the method or use includes a compound of Formula (I), wherein R₂ is C₁₋₆alkyl selected from the group consisting of methyl, ethyl, propyl, butyl, pentyl, and hexyl,
wherein C₁₋₆alkyl contains a chiral carbon having an (R) configuration, and
wherein C₁₋₆alkyl is optionally substituted with one, two, three, or four, independently selected R₂ₐ substituents.

Another aspect of the method or use includes a compound of Formula (I), wherein R₂ is C₁₋₆alkyl selected from the group consisting of methyl, ethyl, propyl, butyl, pentyl, and hexyl,
wherein C₁₋₆alkyl contains a chiral carbon having an (S) configuration, and
wherein C₁₋₆alkyl is optionally substituted with one, two, three, or four, independently selected R₂ₐ substituents.

Another aspect of the method or use includes a compound of Formula (I), wherein R₂ is C₁₋₆alkyl selected from the group consisting of methyl, ethyl, propyl, butyl, and pentyl,
wherein C₁₋₆alkyl optionally contains a chiral carbon having an (R) or (S) configuration, and
wherein C₁₋₆alkyl is optionally substituted with one, two, three, or four, independently selected R₂ₐ substituents.

Another aspect of the method or use includes a compound of Formula (I), wherein R₂ is C₁₋₆alkyl selected from the group consisting of methyl, ethyl, propyl, butyl, and pentyl,
wherein C₁₋₆alkyl contains a chiral carbon having an (R) configuration, and
wherein C₁₋₆alkyl is optionally substituted with one, two, three, or four, independently selected R₂ₐ substituents.

Another aspect of the method or use includes a compound of Formula (I), wherein R₂ is C₁₋₆alkyl selected from the group consisting of methyl, ethyl, propyl, butyl, and pentyl,
wherein C₁₋₆alkyl contains a chiral carbon having an (S) configuration, and
wherein C₁₋₆alkyl is optionally substituted with one, two, three, or four, independently selected R₂ₐ substituents.

Another aspect of the method or use includes a compound of Formula (I), wherein R₂ is heterocyclyl,
wherein heterocyclyl is a 3-7 membered monocyclic carbon atom ring structure radical
containing 1-3 heteroatoms selected from N, O, and S, and
wherein heterocyclyl is optionally substituted with one, two, three, or four independently selected R₂ₐ substituents.

Another aspect of the method or use includes a compound of Formula (I), wherein R₂ is heterocyclyl,
wherein heterocyclyl is a 3-7 membered monocyclic carbon atom ring structure radical containing 1-3 heteroatoms selected from N, O, and S, and
wherein heterocyclyl is optionally substituted with one, two, three, or four independently selected R₂ₐ substituents.

Another aspect of the method or use includes a compound of Formula (I), wherein R₂ is heterocyclyl,
wherein heterocyclyl is a 3-7 membered monocyclic carbon atom ring structure radical containing 1-3 heteroatoms selected from N, O, and S, and
wherein heterocyclyl is optionally substituted with one, two, three, or four independently selected R₂ₐ substituents.

Another aspect of the method or use includes a compound of Formula (I), wherein R₂ is heterocyclyl selected from the group consisting of azetidinyl, oxetanyl, pyrazolidinyl, tetrahydrofuranyl, oxazolidinyl, thiazolidinyl, isothiazolidinyl, pyrrolidinyl, piperidinyl, piperazinyl, 2H-pyranyl, tetrahydropyranyl, morpholinyl, 1,3-oxazinanyl, 1,3-oxazinan-2-on-yl, and azepanyl, and
wherein heterocyclyl is optionally substituted with one, two, three, or four independently selected R₂ₐ substituents.

Another aspect of the method or use includes a compound of Formula (I), wherein R₂ is heterocyclyl selected from the group consisting of azetidinyl and pyrrolidinyl, and
wherein heterocyclyl is optionally substituted with one, two, three, or four independently selected R₂ₐ substituents.

Another aspect of the method or use includes a compound of Formula (I), wherein R₂ is heterocyclyl selected from the group consisting of azetidin-2-yl, azetidin-3-yl, oxetan-2-yl, oxetan-3-yl, pyrazolidin-1-yl, pyrazolidin-2-yl, pyrazolidin-3-yl, pyrazolidin-4-yl, pyrazolidin-5-yl, tetrahydrofuran-1-yl, tetrahydrofuran-2-yl, oxazolidin-2-yl, oxazolidin-4-yl, oxazolidin-5-yl, thiazolidin-2-yl, thiazolidin-4-yl, thiazolidin-5-yl, isothiazolidin-3-yl, isothiazolidin-4-yl, isothiazolidin-5-yl, pyrrolidin-2-yl, pyrrolidin-3-yl, piperidin-1-yl, piperidin-2-yl, piperidin-3-yl, piperidin-4-yl, piperazin-1-yl, piperazin-2-yl, piperazin-3-yl, 2H-pyran-2-yl, 2H-pyran-3-yl, 2H-pyran-4-yl, 2H-pyran-5-yl, 2H-pyran-6-yl, tetrahydropyran-2-yl, tetrahydropyran-3-yl, tetrahydropyran-4-yl, morpholin-2-yl, morpholin-3-yl, morpholin-4-yl, 1,3-oxazinan-2-yl, 1,3-oxazinan-3-yl, 1,3-oxazinan-4-yl, 1,3-oxazinan-2-on-6-yl, azepan-1-yl, azepan-2-yl, azepan-3-yl, and azepan-4-yl, and
wherein heterocyclyl is optionally substituted with one, two, three, or four independently selected R₂ₐ substituents.

Another aspect of the method or use includes a compound of Formula (I), wherein R₂ is heterocyclyl selected from the group consisting of azetidin-3-yl and pyrrolidin-3-yl, and
wherein heterocyclyl is optionally substituted with one, two, three, or four independently selected R₂ₐ substituents.

One aspect of the method or use includes a compound of Formula (I), wherein R₂ₐ is independently selected from the group consisting of cyano, halo, hydroxy, oxo, C₁₋₆alkyl, halo-C₁₋₆alkyl, deutero-C₁₋₆alkyl, C₂₋₆alkenyl, C₂₋₆alkynyl, C₁₋₆alkoxy, halo-C₁₋₆alkoxy, carboxyl, amino, C₁₋₆alkyl-amino, halo-C₁₋₆alkyl-amino, deutero-C₁₋₆alkyl-amino, (C₁₋₆alkyl)₂-amino, C₃₋₁₀cycloalkyl-amino, phenyl-amino, heterocyclyl-amino, heteroaryl-amino, C₁₋₆alkyl-thio, C₁₋₆alkyl-sulfonyl, C₃₋₁₀cycloalkyl, phenyl, heterocyclyl, and heteroaryl,
wherein heterocyclyl is a 3-7 membered monocyclic carbon atom ring structure radical containing 1-3 heteroatoms selected from N, O, and S,
wherein heteroaryl is a 5-8 membered monocyclic or bicyclic aromatic carbon atom ring structure radical containing 1-3 heteroatoms selected from N, O, and S, and
wherein each instance of C₃₋₁₀cycloalkyl, phenyl, heterocyclyl or heteroaryl is optionally substituted with one, two, three or four independently selected R_{2a′} substituents.

Another aspect of the method or use includes a compound of Formula (I), wherein R₂ₐ is independently selected from the group consisting of halo, hydroxy, C₁₋₆alkyl, C₁₋₆alkoxy, amino, C₁₋₆alkyl-amino, C₃₋₁₀cycloalkyl-amino, C₃₋₁₀cycloalkyl, or heterocyclyl,
wherein each instance of C₃₋₁₀cycloalkyl or heterocyclyl is optionally substituted with one, two, three or four independently selected R_{2a′} substituents.

Another aspect of the method or use includes a compound of Formula (I), wherein R₂ₐ is halo selected from the group consisting of fluoro, chloro, bromo, and iodo.

Another aspect of the method or use includes a compound of Formula (I), wherein R₂ₐ is fluoro.

Another aspect of the method or use includes a compound of Formula (I), wherein R₂ₐ is hydroxy.

Another aspect of the method or use includes a compound of Formula (I), wherein R₂ₐ is C₁₋₆alkyl selected from the group consisting of methyl, ethyl, propyl, butyl, pentyl, and hexyl.

Another aspect of the method or use includes a compound of Formula (I), wherein R₂ₐ is methyl.

Another aspect of the method or use includes a compound of Formula (I), wherein R₂ₐ is C₁₋₆alkoxy selected from the group consiting of methoxy, ethoxy, propoxy, isopropoxy, butoxy, and tert-butoxy.

Another aspect of the method or use includes a compound of Formula (I), wherein R₂ₐ is methoxy.

Another aspect of the method or use includes a compound of Formula (I), wherein R₂ₐ is amino.

Another aspect of the method or use includes a compound of Formula (I), wherein R₂ₐ is C₁₋₆alkyl-amino, and wherein C₁₋₆alkyl is selected from the group consisting of methyl, ethyl, propyl, isopropyl, butyl, sec-butyl, isobutyl, and tert-butyl.

Another aspect of the method or use includes a compound of Formula (I), wherein R₂ₐ is methyl-amino.

Another aspect of the method or use includes a compound of Formula (I), wherein R₂ₐ is C₃₋₁₀cycloalkyl-amino, wherein C₃₋₁₀cycloalkyl is selected from the group consisting of cyclopropyl, cyclobutyl, cyclopentyl, cyclohexyl, cycloheptyl, and cyclooctyl, and wherein C₃₋₁₀ cycloalkyl is optionally substituted with one, two, three or four independently selected R_{2a′} substituents.

Another aspect of the method or use includes a compound of Formula (I), wherein R₂ₐ is cyclobutyl-amino.

Another aspect of the method or use includes a compound of Formula (I), wherein R₂ₐ is C₃₋₁₀cycloalkyl, wherein C₃₋₁₀cycloalkyl is selected from cyclopropyl, cyclobutyl, cyclopentyl, cyclohexyl, cycloheptyl, and cyclooctyl, and wherein C₃₋₁₀cycloalkyl is optionally substituted with one, two, three or four independently selected R_{2a′} substituents.

Another aspect includes a compound of Formula (I), wherein R₂ₐ is cyclopropyl, optionally substituted with one, two, three or four independently selected R_{2a′} substituents.

Another aspect of the method or use includes a compound of Formula (I), wherein R₂ₐ is heterocyclyl selected from the group consisting of azetidinyl, oxetanyl, pyrazolidinyl, tetrahydrofuranyl, oxazolidinyl, thiazolidinyl, isothiazolidinyl, pyrrolidinyl, piperidinyl, piperazinyl, 2H-pyranyl, tetrahydropyranyl, morpholinyl, 1,3-oxazinanyl, 1,3-oxazinan-2-on-yl, and azepanyl, and
wherein heterocyclyl is optionally substituted with one, two, three, or four independently selected R_{2a′} substituents.

Another aspect of the method or use includes a compound of Formula (I), wherein R₂ₐ is 1,3-oxazinan-2-on-yl.

Another aspect of the method or use includes a compound of Formula (I), wherein R₂ₐ is heterocyclyl selected from the group consisting of azetidin-2-yl, azetidin-3-yl, oxetan-2-yl, oxetan-3-yl, pyrazolidin-1-yl, pyrazolidin-2-yl, pyrazolidin-3-yl, pyrazolidin-4-yl, pyrazolidin-5-yl, tetrahydrofuran-1-yl, tetrahydrofuran-2-yl, oxazolidin-2-yl, oxazolidin-4-yl, oxazolidin-5-yl, thiazolidin-2-yl, thiazolidin-4-yl, thiazolidin-5-yl, isothiazolidin-3-yl, isothiazolidin-4-yl, isothiazolidin-5-yl, pyrrolidin-2-yl, pyrrolidin-3-yl, piperidin-1-yl, piperidin-2-yl, piperidin-3-yl, piperidin-4-yl, piperazin-1-yl, piperazin-2-yl, piperazin-3-yl, 2H-pyran-2-yl, 2H-pyran-3-yl, 2H-pyran-4-yl, 2H-pyran-5-yl, 2H-pyran-6-yl, tetrahydropyran-2-yl, tetrahydropyran-3-yl, tetrahydropyran-4-yl, morpholin-2-yl, morpholin-3-yl, morpholin-4-yl, 1,3-oxazinan-2-yl, 1,3-oxazinan-3-yl, 1,3-oxazinan-4-yl, 1,3-oxazinan-2-on-6-yl, azepan-1-yl, azepan-2-yl, azepan-3-yl, and azepan-4-yl, and wherein heterocyclyl is optionally substituted with one, two, three, or four independently selected R_{2a′} substituents.

Another aspect of the method or use includes a compound of Formula (I), wherein R₂ₐ is 1,3-oxazinan-2-on-6-yl.

One aspect includes a compound of Formula (I), wherein R₃ is selected from the group consisting of hydrogen, cyano, halo, hydroxy, C₁₋₆alkyl, halo-C₁₋₆alkyl, C₁₋₆alkoxy, amino, C₁₋₆alkyl-amino, (C₁₋₆alkyl)₂-amino, C₃₋₁₀cycloalkyl, phenyl, heterocyclyl, and heteroaryl,
wherein heterocyclyl is a 3-7 membered monocyclic carbon atom ring structure radical containing 1-3 heteroatoms selected from N, O, and S, and
wherein heteroaryl is a 5-8 membered monocyclic or bicyclic aromatic carbon atom ring structure radical containing 1-3 heteroatoms selected from N, O, and S, and
wherein each instance of C₁₋₆alkyl, C₃₋₁₀cycloalkyl, phenyl, heterocyclyl, or heteroaryl is optionally substituted with one, two, three, or four independently selected R₃ₐ substituents.

Another aspect includes a compound of Formula (I), wherein R₃ is selected from the group consisting of hydrogen, cyano, halo, C₁₋₆alkyl, C₁₋₆alkoxy, C₃₋₁₀cycloalkyl, and phenyl, and
wherein C₁₋₆alkyl, C₃₋₁₀cycloalkyl, or phenyl is optionally substituted with one, two, three, or four independently selected R₃ₐ substituents.

Another aspect of the method or use includes a compound of Formula (I), wherein R₃ is hydrogen.

Another aspect of the method or use includes a compound of Formula (I), wherein R₃ is cyano.

Another aspect of the method or use includes a compound of Formula (I), wherein R₃ is halo selected from the group consisting of fluoro, chloro, bromo, and iodo.

Another aspect of the method or use includes a compound of Formula (I), wherein R₃ is bromo.

Another aspect of the method or use includes a compound of Formula (I), wherein R₃ is hydroxy.

Another aspect of the method or use includes a compound of Formula (I), wherein R₃ is selected from the group consisting of methyl, ethyl, propyl, butyl, pentyl, and hexyl, and
wherein C₁₋₆alkyl is optionally substituted with one, two, three, or four independently selected R₃ₐ substituents.

Another aspect of the method or use includes a compound of Formula (I), wherein R₃ is C₁₋₆alkyl selected from the group consisting of methyl and ethyl, and
wherein C₁₋₆alkyl is optionally substituted with one, two, three, or four independently selected R₃ₐ substituents.

Another aspect of the method or use includes a compound of Formula (I), wherein R₃ is C₁₋₆alkoxy selected from the group consisting of methoxy, ethoxy, propoxy, isopropoxy, butoxy, and tert-butoxy.

Another aspect of the method or use includes a compound of Formula (I), wherein R₃ is methoxy.

Another aspect of the method or use includes a compound of Formula (I), wherein R₃ is C₃₋₁₀cycloalkyl selected from the group consiting of cyclopropyl, cyclobutyl, cyclopentyl, cyclohexyl, cycloheptyl, and cyclooctyl, and
wherein C₃₋₁₀cycloalkyl is optionally substituted with one, two, three, or four independently selected R₃ₐ substituents.

Another aspect of the method or use includes a compound of Formula (I), wherein R₃ is cyclopropyl, optionally substituted with one, two, three, or four independently selected R₃ₐ substituents.

Another aspect of the method or use includes a compound of Formula (I), wherein R₃ is phenyl, optionally substituted with one, two, three, or four independently selected R₃ₐ substituents.

One aspect of the method or use includes a compound of Formula (I), wherein R₃ₐ is selected from the group consisting of cyano, halo, hydroxy, C₁₋₆alkyl, halo-C₁₋₆alkyl, and C₁₋₆alkoxy.

Another aspect of the method or use includes a compound of Formula (I), wherein R₃ₐ is selected from the group consisting of halo and C₁₋₆alkoxy.

Another aspect of the method or use includes a compound of Formula (I), wherein R₃ₐ is halo selected from the group consisting of fluoro, chloro, bromo, and iodo.

Another aspect of the method or use includes a compound of Formula (I), wherein R₃ₐ is chloro.

Another aspect of the method or use includes a compound of Formula (I), wherein R₃ₐ is C₁₋₆alkoxy selected from the group consisting of methoxy, ethoxy, propoxy, isopropoxy, butoxy, and tert-butoxy.

Another aspect of the method or use includes a compound of Formula (I), wherein R₃ₐ is methoxy.

One aspect of the method or use includes a compound of Formula (I), wherein R₄ is selected from the group consisting of hydrogen, cyano, halo, hydroxy, C₁₋₆alkyl, halo-C₁₋₆alkyl, C₁₋₆alkoxy, carbamoyl, C₃₋₁₀cycloalkyl, phenyl, and heterocyclyl, and
wherein heterocyclyl is a 3-7 membered monocyclic carbon atom ring structure radical containing 1-3 heteroatoms selected from N, O, and S.

Another aspect of the method or use includes a compound of Formula (I), wherein R₄ is selected from the group consisting of hydrogen, cyano, halo, C₁₋₆alkyl, halo-C₁₋₆alkyl, carbamoyl, C₃₋₁₀cycloalkyl, and phenyl.

Another aspect of the method or use includes a compound of Formula (I), wherein R₄ is hydrogen.

Another aspect of the method or use includes a compound of Formula (I), wherein R₄ is cyano.

Another aspect of the method or use includes a compound of Formula (I), wherein R₄ is halo selected from the group consisting of fluoro, chloro, bromo, and iodo.

Another aspect of the method or use includes a compound of Formula (I), wherein R₄ is halo selected the group consisting of chloro and bromo.

Another aspect of the method or use includes a compound of Formula (I), wherein R₄ is C₁₋₆alkyl selected from the group consisting of methyl, ethyl, propyl, butyl, pentyl, and hexyl.

Another aspect of the method or use includes a compound of Formula (I), wherein R₄ is C₁₋₆alkyl selected from the group consisting of methyl and ethyl.

Another aspect of the method or use includes a compound of Formula (I), wherein R₄ is halo-C₁₋₆alkyl, wherein C₁₋₆alkyl is selected from the group consisting of methyl, ethyl, propyl, butyl, pentyl, and hexyl, and
wherein C₁₋₆alkyl is partially or completely substituted with one or more halogen atoms where allowed by available valences.

Another aspect of the method or use includes a compound of Formula (I), wherein R₄ is halo-C₁₋₆alkyl, and wherein C₁₋₆alkyl is methyl substituted with three fluorine atoms.

Another aspect of the method or use includes a compound of Formula (I), wherein R₄ is carbamoyl.

Another aspect of the method or use includes a compound of Formula (I), wherein R₄ is C₃₋₁₀cycloalkyl selected from the group consisting of cyclopropyl, cyclobutyl, cyclopentyl, cyclohexyl, cycloheptyl, and cyclooctyl.

Another aspect of the method or use includes a compound of Formula (I), wherein R₄ is cyclopropyl.

Another aspect of the method or use includes a compound of Formula (I), wherein R₄ is phenyl.

One aspect of the method or use includes a compound of Formula (I) or a form thereof includes a compound selected from the group consisting of.

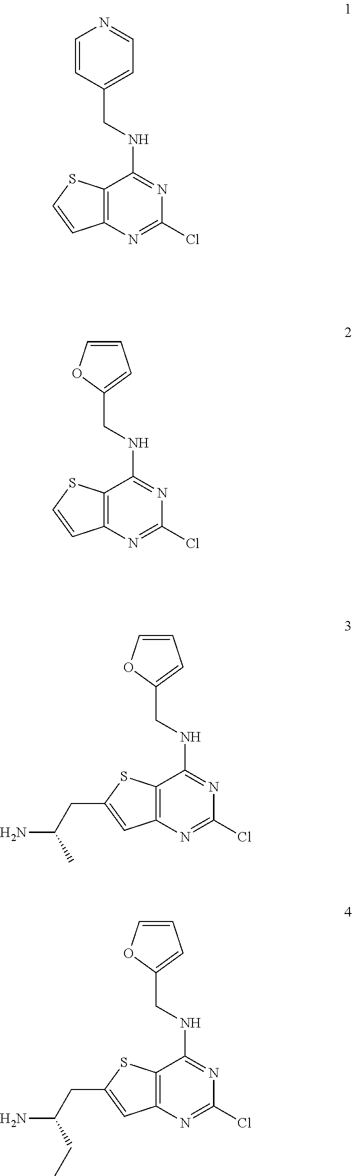 [SMILES: Clc1nc(NCc2ccncc2)c2sccc2n1 ; Clc1nc(NCc2ccco2)c2sccc2n1 ; C[C@H](N)Cc1cc2nc(Cl)nc(NCc3ccco3)c2s1 ; CC[C@H](N)Cc1cc2nc(Cl)nc(NCc3ccco3)c2s1]

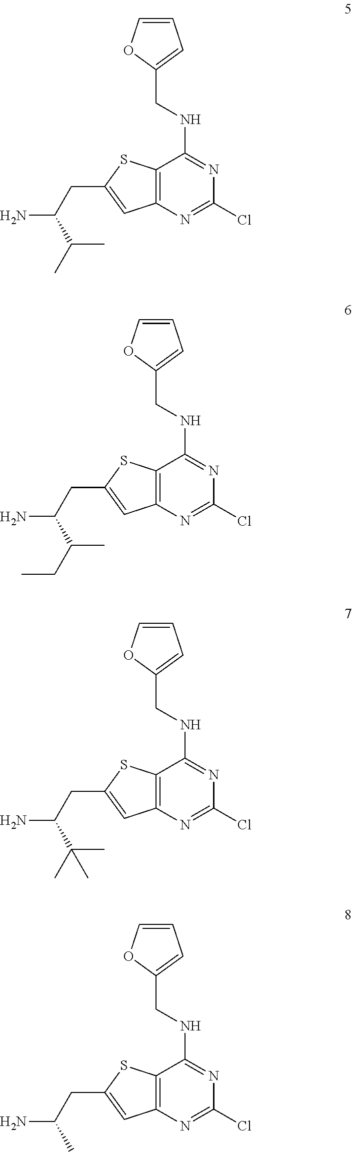 [SMILES: CC(C)[C@H](N)Cc1cc2nc(Cl)nc(NCc3ccco3)c2s1 ; CCC(C)[C@H](N)Cc1cc2nc(Cl)nc(NCc3ccco3)c2s1 ; CC(C)(C)[C@H](N)Cc1cc2nc(Cl)nc(NCc3ccco3)c2s1 ; C[C@H](N)Cc1cc2nc(Cl)nc(NCc3ccco3)c2s1]

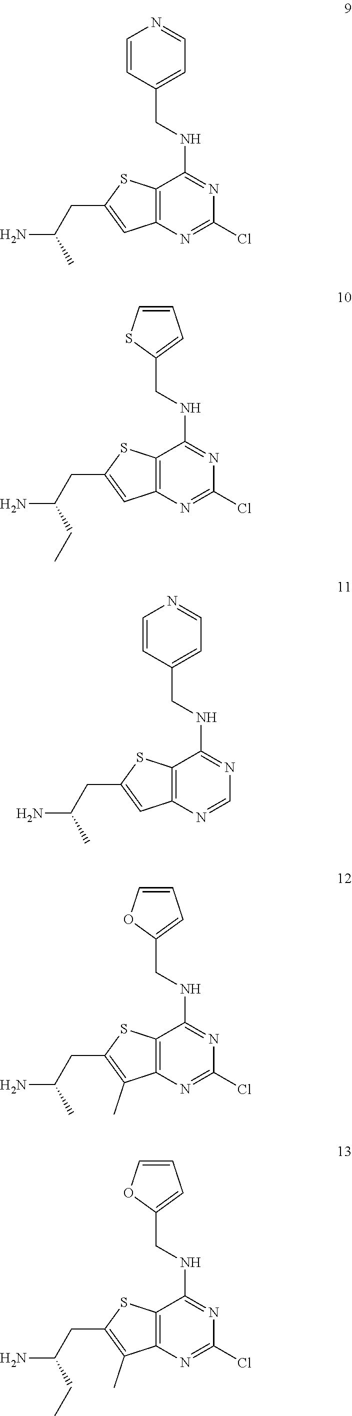 [SMILES: C[C@H](N)Cc1cc2nc(Cl)nc(NCc3ccncc3)c2s1 ; CC[C@H](N)Cc1cc2nc(Cl)nc(NCc3cccs3)c2s1 ; C[C@H](N)Cc1cc2ncnc(NCc3ccncc3)c2s1 ; Cc1c(C[C@H](C)N)sc2c(NCc3ccco3)nc(Cl)nc12 ; CC[C@H](N)Cc1sc2c(NCc3ccco3)nc(Cl)nc2c1C]

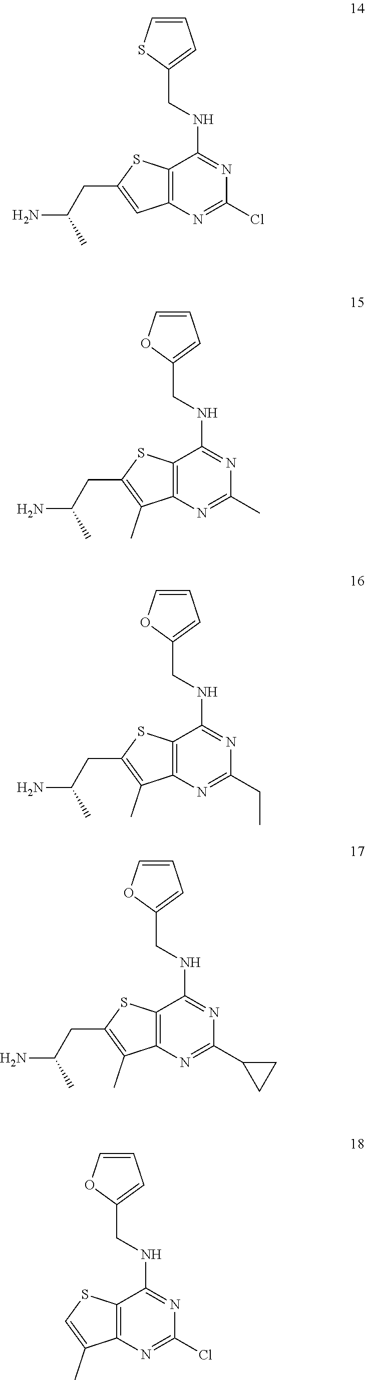

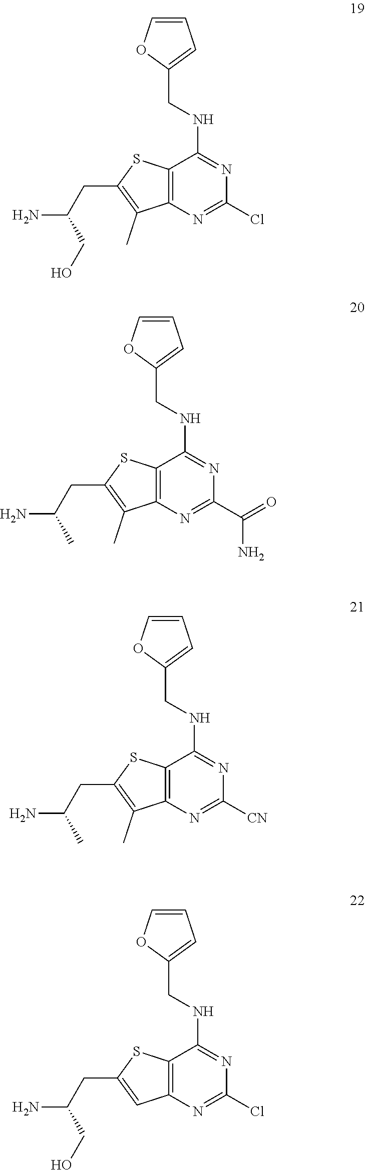 [SMILES: Cc1c(C[C@@H](N)CO)sc2c(NCc3ccco3)nc(Cl)nc12 ; Cc1c(C[C@H](C)N)sc2c(NCc3ccco3)nc(C(N)=O)nc12 ; Cc1c(C[C@H](C)N)sc2c(NCc3ccco3)nc(C#N)nc12 ; N[C@@H](CO)Cc1cc2nc(Cl)nc(NCc3ccco3)c2s1]

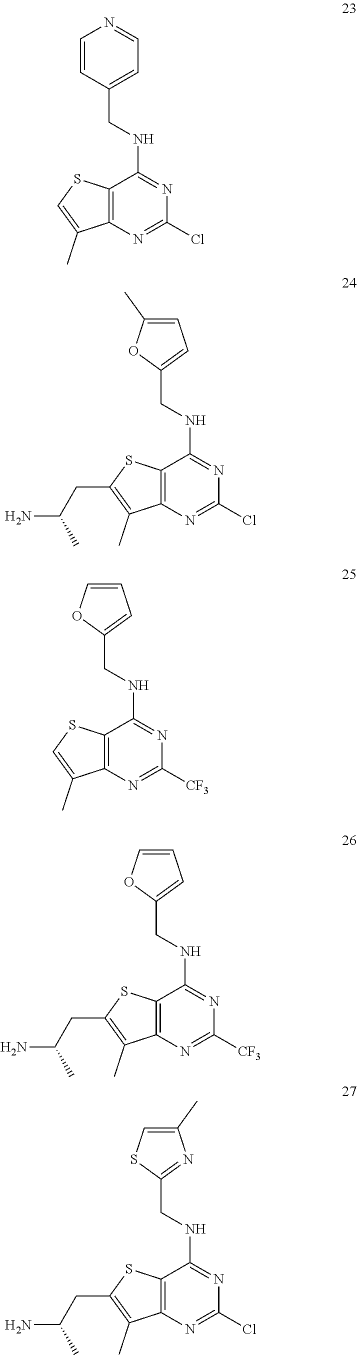

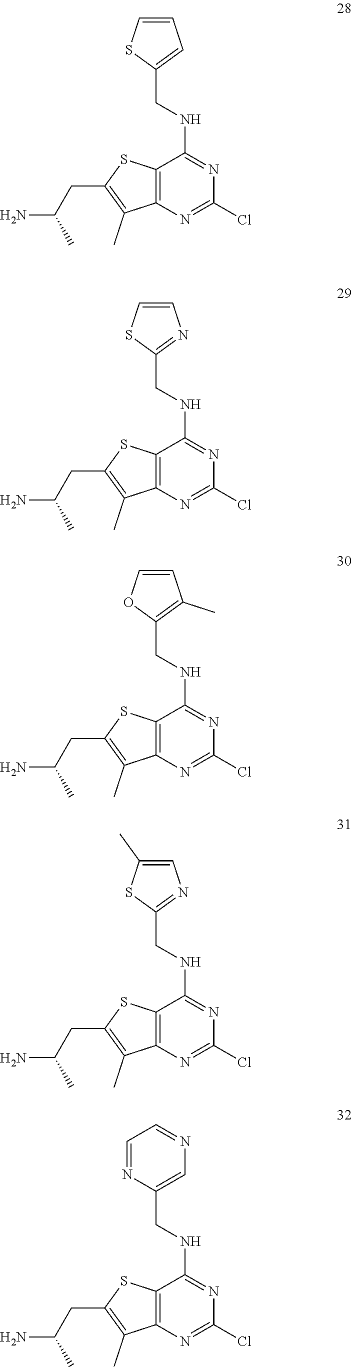

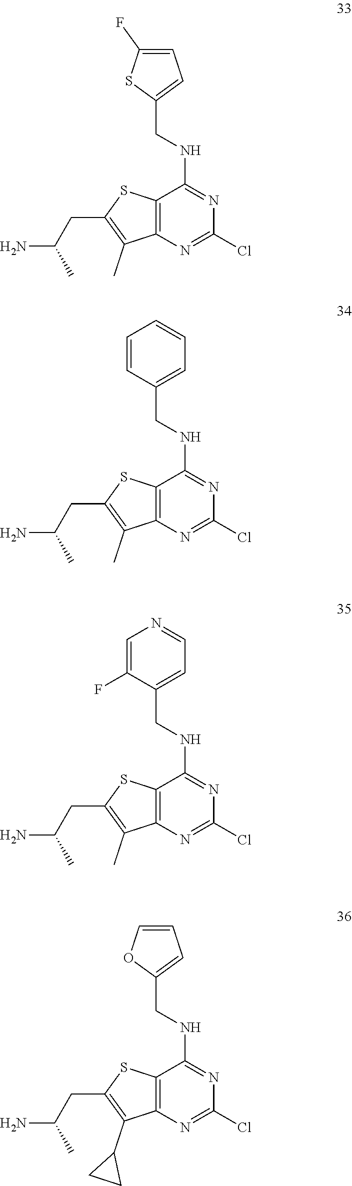 [SMILES: Cc1c(C[C@H](C)N)sc2c(NCc3ccc(F)s3)nc(Cl)nc12 ; Cc1c(C[C@H](C)N)sc2c(NCc3ccccc3)nc(Cl)nc12 ; Cc1c(C[C@H](C)N)sc2c(NCc3ccncc3F)nc(Cl)nc12 ; C[C@H](N)Cc1sc2c(NCc3ccco3)nc(Cl)nc2c1C1CC1]

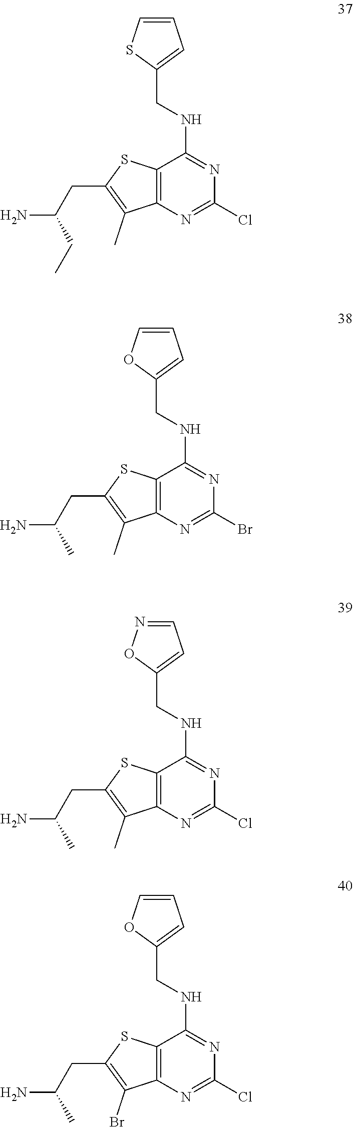 [SMILES: CC[C@H](N)Cc1sc2c(NCc3cccs3)nc(Cl)nc2c1C ; Cc1c(C[C@H](C)N)sc2c(NCc3ccco3)nc(Br)nc12 ; Cc1c(C[C@H](C)N)sc2c(NCc3ccno3)nc(Cl)nc12 ; C[C@H](N)Cc1sc2c(NCc3ccco3)nc(Cl)nc2c1Br]

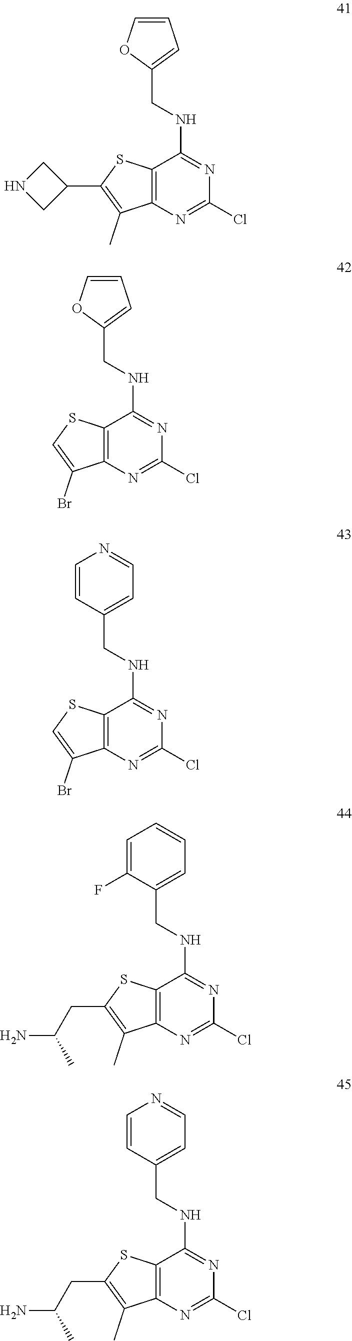

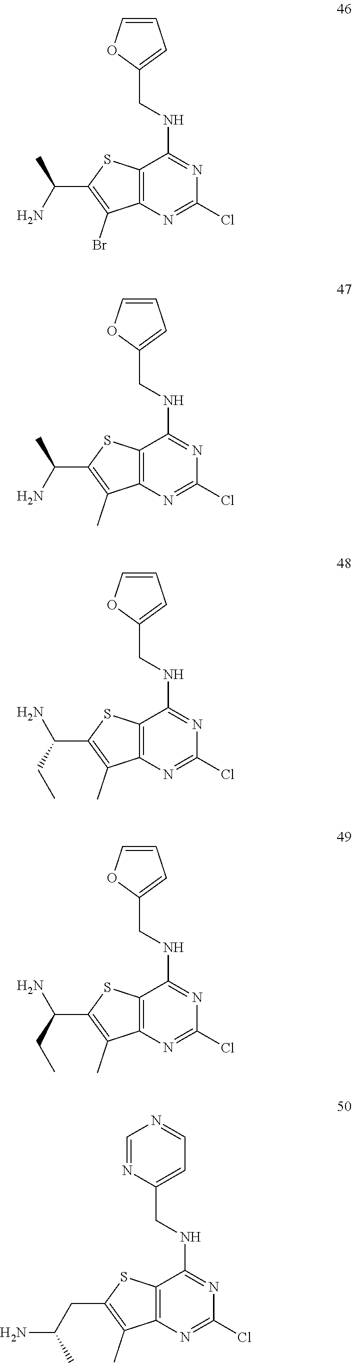 [SMILES: C[C@H](N)c1sc2c(NCc3ccco3)nc(Cl)nc2c1Br ; Cc1c([C@H](C)N)sc2c(NCc3ccco3)nc(Cl)nc12 ; CC[C@H](N)c1sc2c(NCc3ccco3)nc(Cl)nc2c1C ; CC[C@@H](N)c1sc2c(NCc3ccco3)nc(Cl)nc2c1C ; Cc1c(C[C@H](C)N)sc2c(NCc3ccncn3)nc(Cl)nc12]

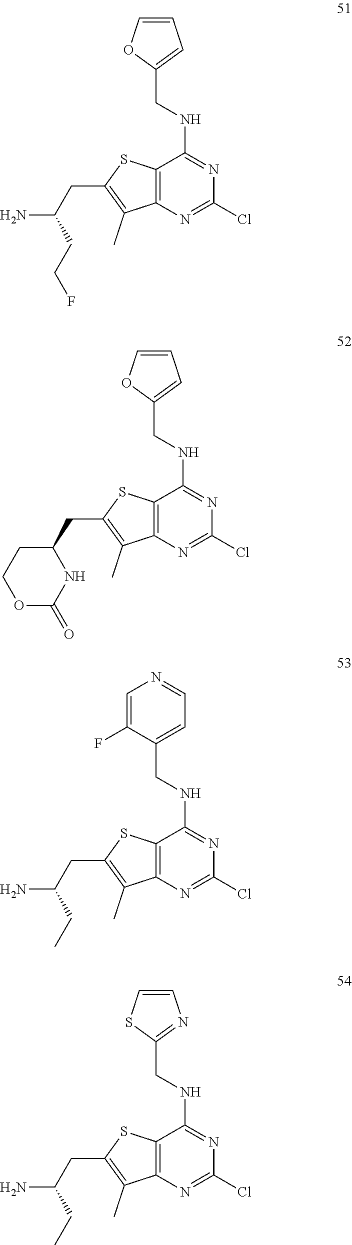 [SMILES: Cc1c(C[C@@H](N)CCF)sc2c(NCc3ccco3)nc(Cl)nc12 ; Cc1c(C[C@@H]2CCOC(=O)N2)sc2c(NCc3ccco3)nc(Cl)nc12 ; CC[C@H](N)Cc1sc2c(NCc3ccncc3F)nc(Cl)nc2c1C ; CC[C@H](N)Cc1sc2c(NCc3nccs3)nc(Cl)nc2c1C]

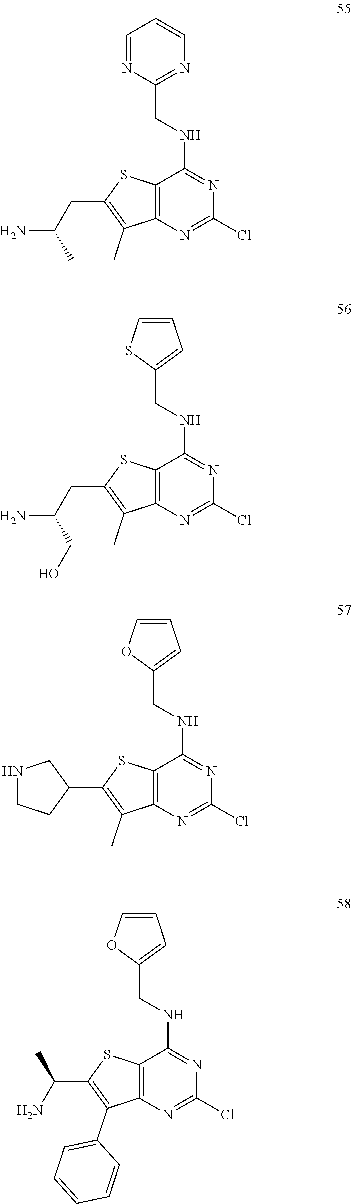 [SMILES: Cc1c(C[C@H](C)N)sc2c(NCc3ncccn3)nc(Cl)nc12 ; Cc1c(C[C@@H](N)CO)sc2c(NCc3cccs3)nc(Cl)nc12 ; Cc1c(C2CCNC2)sc2c(NCc3ccco3)nc(Cl)nc12 ; C[C@H](N)c1sc2c(NCc3ccco3)nc(Cl)nc2c1-c1ccccc1]

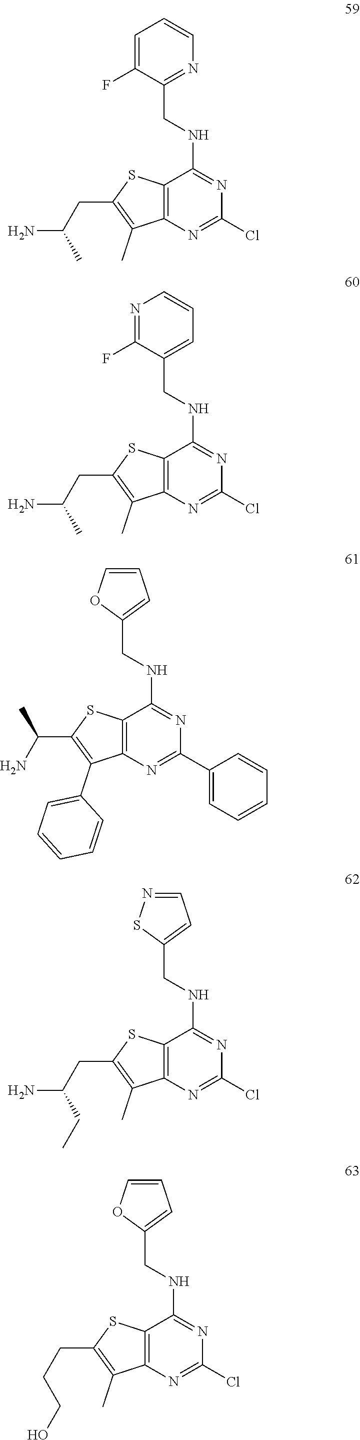

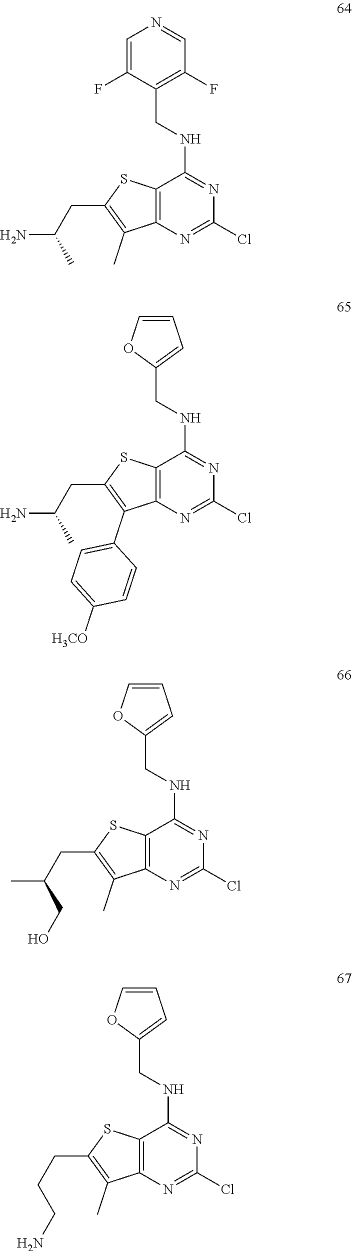 [SMILES: Cc1c(C[C@H](C)N)sc2c(NCc3c(F)cncc3F)nc(Cl)nc12 ; COc1ccc(-c2c(C[C@H](C)N)sc3c(NCc4ccco4)nc(Cl)nc23)cc1 ; Cc1c(C[C@H](C)CO)sc2c(NCc3ccco3)nc(Cl)nc12 ; Cc1c(CCCN)sc2c(NCc3ccco3)nc(Cl)nc12]

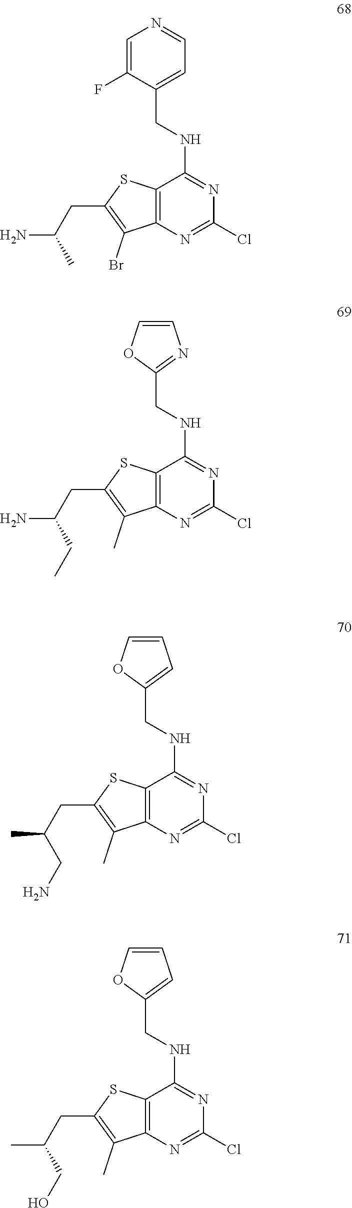 [SMILES: C[C@H](N)Cc1sc2c(NCc3ccncc3F)nc(Cl)nc2c1Br ; CC[C@H](N)Cc1sc2c(NCc3ncco3)nc(Cl)nc2c1C ; Cc1c(C[C@H](C)CN)sc2c(NCc3ccco3)nc(Cl)nc12 ; Cc1c(C[C@@H](C)CO)sc2c(NCc3ccco3)nc(Cl)nc12]

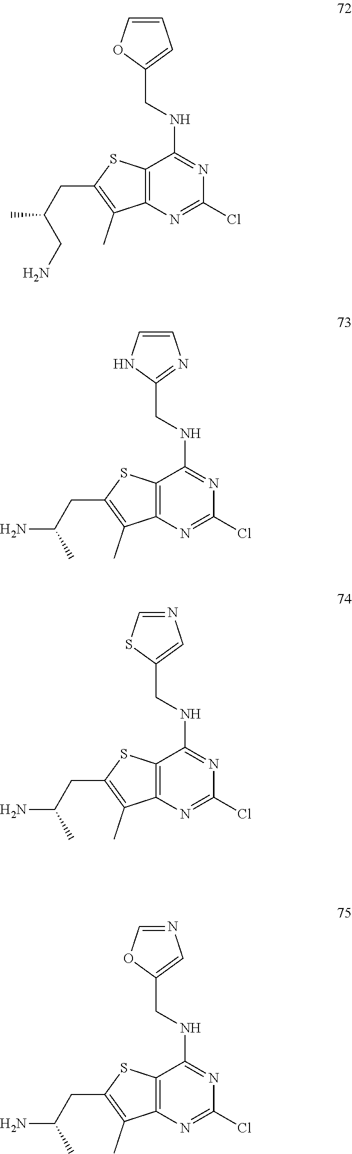 [SMILES: Cc1c(C[C@@H](C)CN)sc2c(NCc3ccco3)nc(Cl)nc12 ; Cc1c(C[C@H](C)N)sc2c(NCc3ncc[nH]3)nc(Cl)nc12 ; Cc1c(C[C@H](C)N)sc2c(NCc3cncs3)nc(Cl)nc12 ; Cc1c(C[C@H](C)N)sc2c(NCc3cnco3)nc(Cl)nc12]

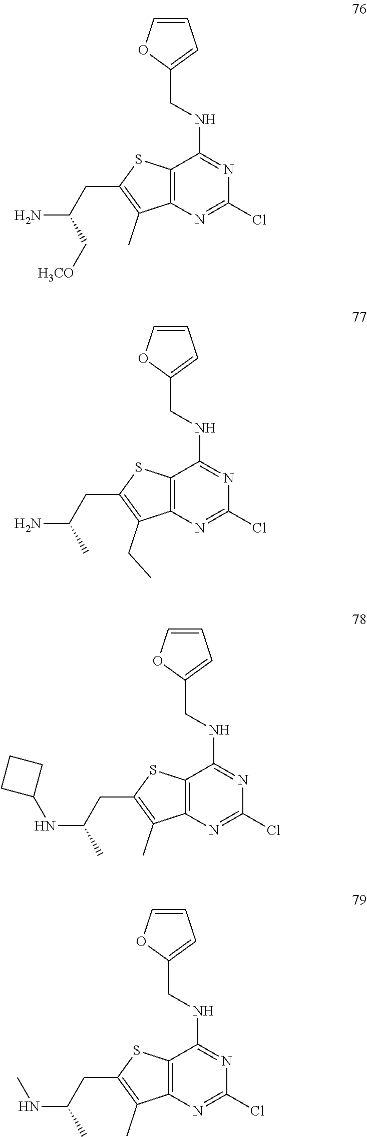 [SMILES: COC[C@H](N)Cc1sc2c(NCc3ccco3)nc(Cl)nc2c1C ; CCc1c(C[C@H](C)N)sc2c(NCc3ccco3)nc(Cl)nc12 ; Cc1c(C[C@H](C)NC2CCC2)sc2c(NCc3ccco3)nc(Cl)nc12 ; CN[C@@H](C)Cc1sc2c(NCc3ccco3)nc(Cl)nc2c1C]

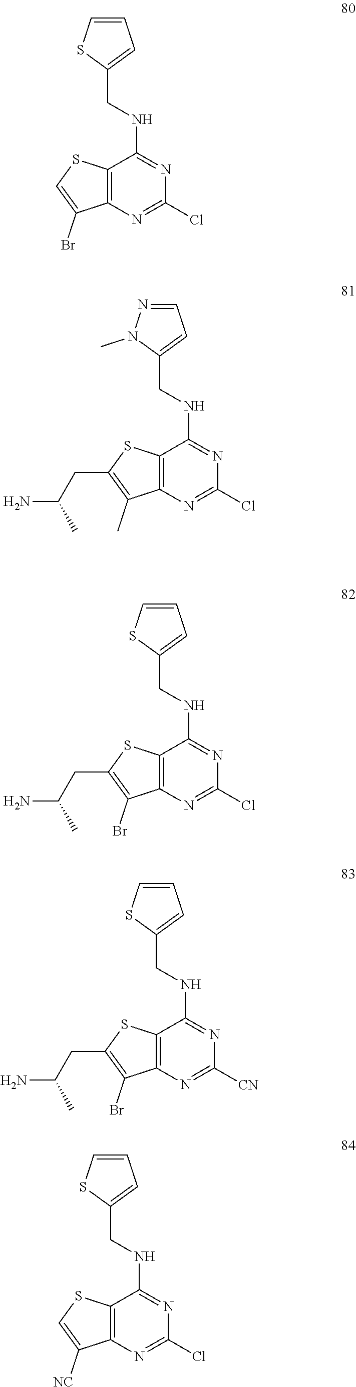 [SMILES: Clc1nc(NCc2cccs2)c2scc(Br)c2n1 ; Cc1c(C[C@H](C)N)sc2c(NCc3ccnn3C)nc(Cl)nc12 ; C[C@H](N)Cc1sc2c(NCc3cccs3)nc(Cl)nc2c1Br ; C[C@H](N)Cc1sc2c(NCc3cccs3)nc(C#N)nc2c1Br ; N#Cc1csc2c(NCc3cccs3)nc(Cl)nc12]

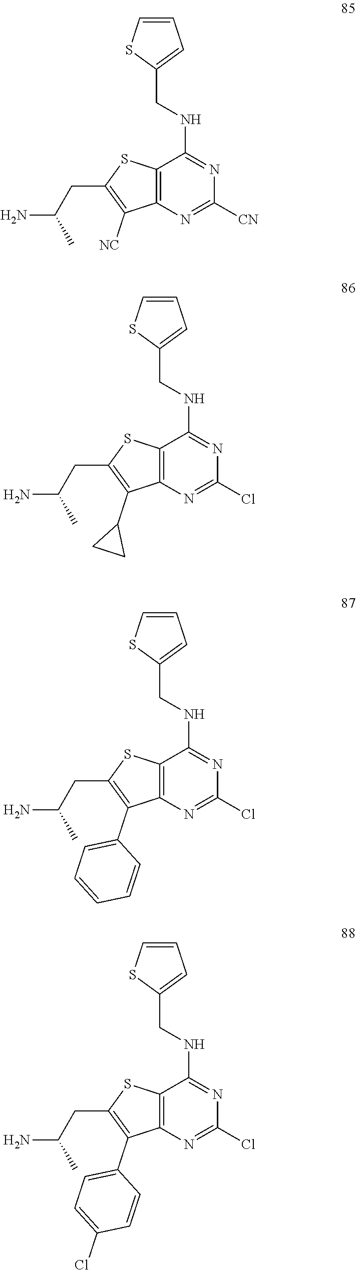 [SMILES: C[C@H](N)Cc1sc2c(NCc3cccs3)nc(C#N)nc2c1C#N ; C[C@H](N)Cc1sc2c(NCc3cccs3)nc(Cl)nc2c1C1CC1 ; C[C@H](N)Cc1sc2c(NCc3cccs3)nc(Cl)nc2c1-c1ccccc1 ; C[C@H](N)Cc1sc2c(NCc3cccs3)nc(Cl)nc2c1-c1ccc(Cl)cc1]

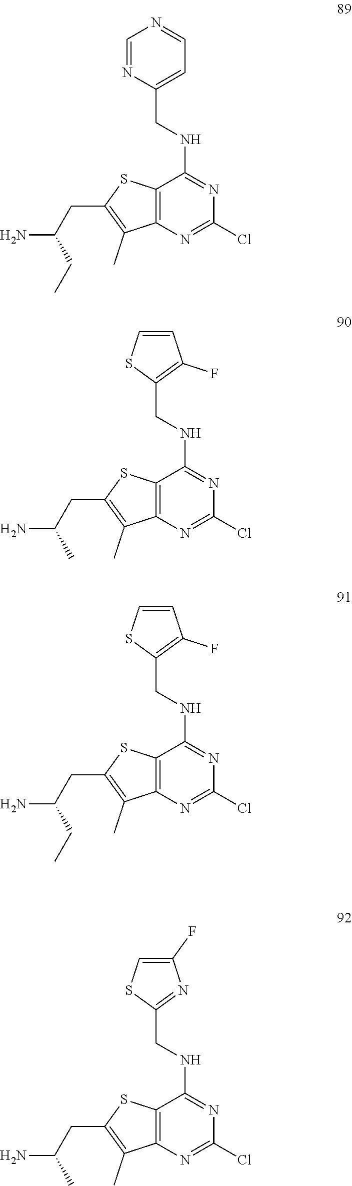 [SMILES: CC[C@H](N)Cc1sc2c(NCc3ccncn3)nc(Cl)nc2c1C ; Cc1c(C[C@H](C)N)sc2c(NCc3sccc3F)nc(Cl)nc12 ; CC[C@H](N)Cc1sc2c(NCc3sccc3F)nc(Cl)nc2c1C ; Cc1c(C[C@H](C)N)sc2c(NCc3nc(F)cs3)nc(Cl)nc12]

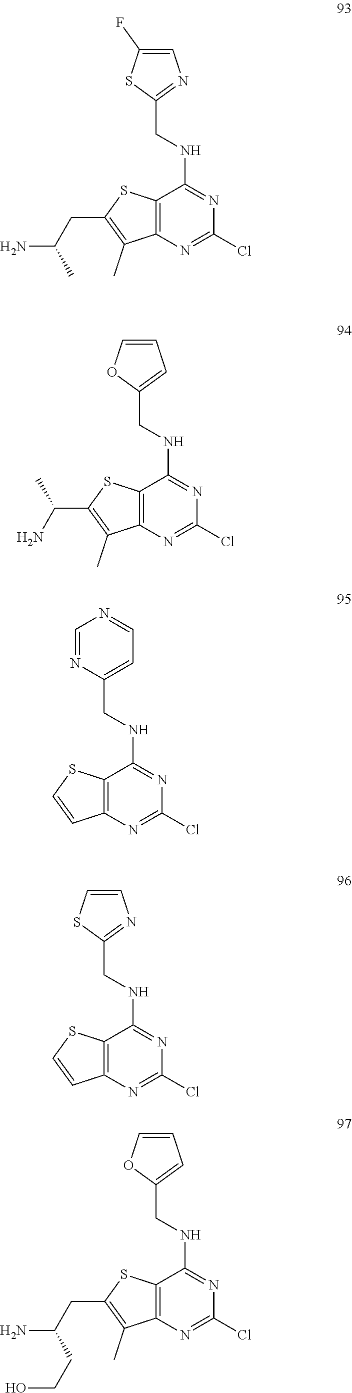

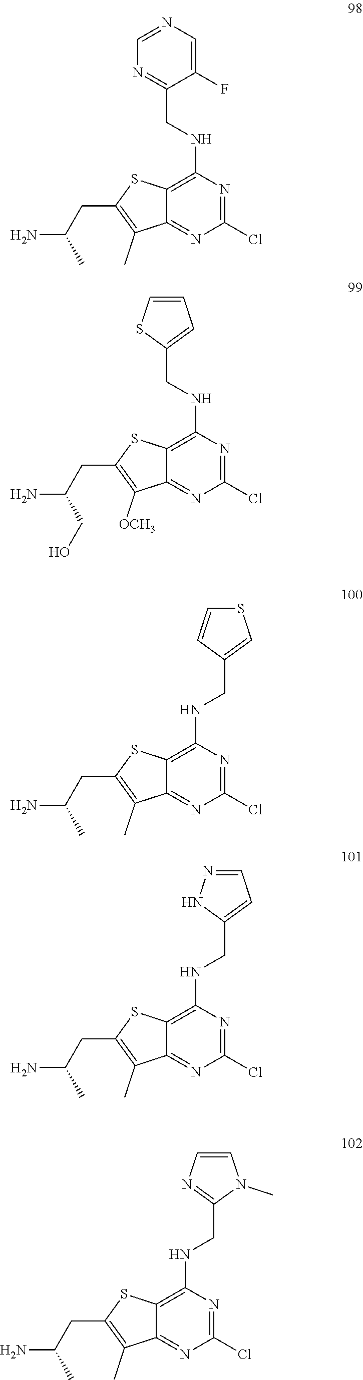

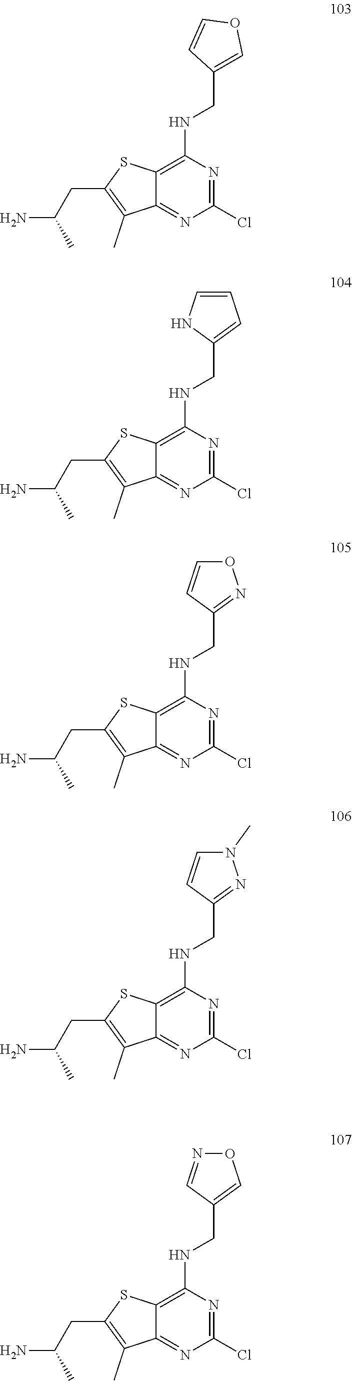 [SMILES: Cc1c(C[C@H](C)N)sc2c(NCc3ccoc3)nc(Cl)nc12 ; Cc1c(C[C@H](C)N)sc2c(NCc3ccc[nH]3)nc(Cl)nc12 ; Cc1c(C[C@H](C)N)sc2c(NCc3ccon3)nc(Cl)nc12 ; Cc1c(C[C@H](C)N)sc2c(NCc3ccn(C)n3)nc(Cl)nc12 ; Cc1c(C[C@H](C)N)sc2c(NCc3cnoc3)nc(Cl)nc12]

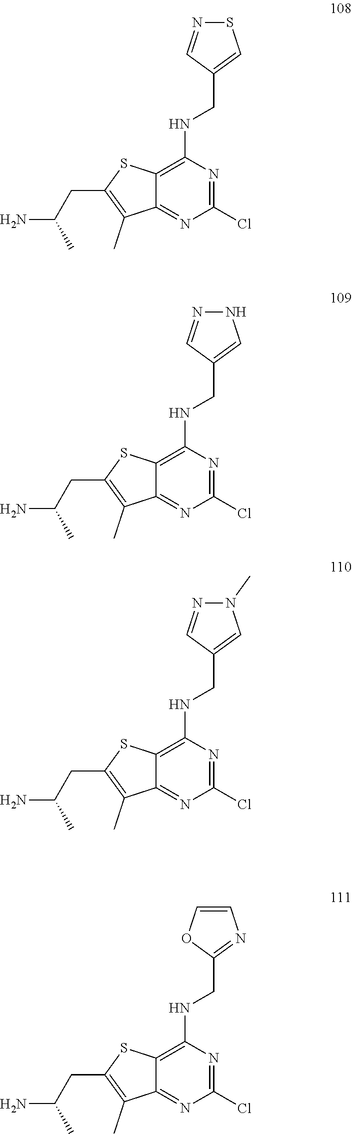 [SMILES: Cc1c(C[C@H](C)N)sc2c(NCc3cnsc3)nc(Cl)nc12 ; Cc1c(C[C@H](C)N)sc2c(NCc3cn[nH]c3)nc(Cl)nc12 ; Cc1c(C[C@H](C)N)sc2c(NCc3cnn(C)c3)nc(Cl)nc12 ; Cc1c(C[C@H](C)N)sc2c(NCc3ncco3)nc(Cl)nc12]

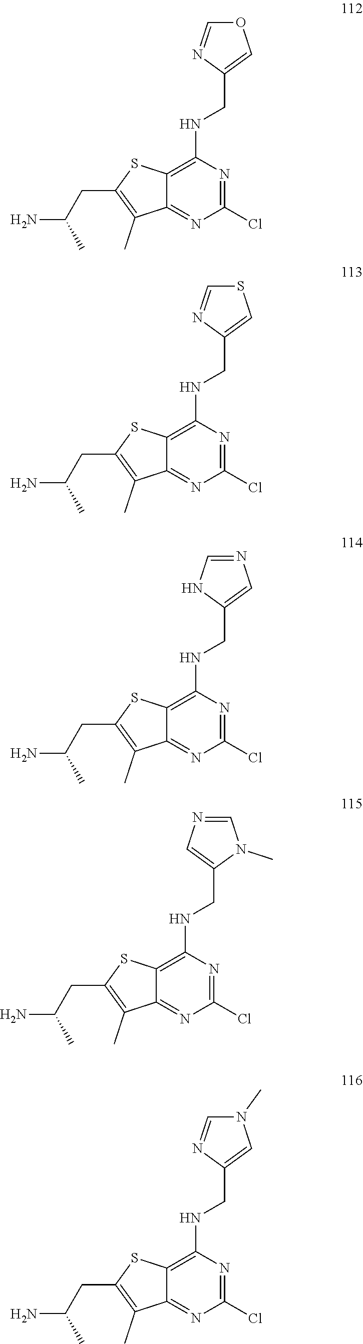 [SMILES: Cc1c(C[C@H](C)N)sc2c(NCc3cocn3)nc(Cl)nc12 ; Cc1c(C[C@H](C)N)sc2c(NCc3cscn3)nc(Cl)nc12 ; Cc1c(C[C@H](C)N)sc2c(NCc3cnc[nH]3)nc(Cl)nc12 ; Cc1c(C[C@H](C)N)sc2c(NCc3cncn3C)nc(Cl)nc12 ; Cc1c(C[C@H](C)N)sc2c(NCc3cn(C)cn3)nc(Cl)nc12]

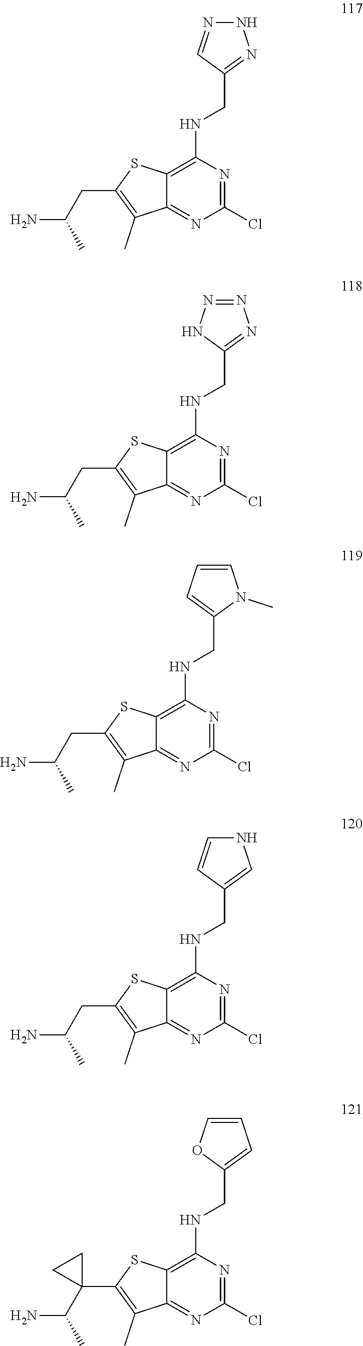 [SMILES: Cc1c(C[C@H](C)N)sc2c(NCc3cn[nH]n3)nc(Cl)nc12 ; Cc1c(C[C@H](C)N)sc2c(NCc3nnn[nH]3)nc(Cl)nc12 ; Cc1c(C[C@H](C)N)sc2c(NCc3cccn3C)nc(Cl)nc12 ; Cc1c(C[C@H](C)N)sc2c(NCc3cc[nH]c3)nc(Cl)nc12 ; Cc1c(C2([C@H](C)N)CC2)sc2c(NCc3ccco3)nc(Cl)nc12]

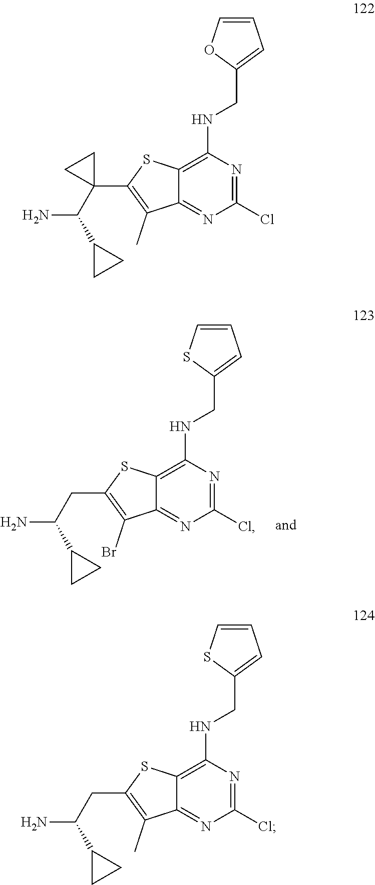 [SMILES: Cc1c(C2([C@@H](N)C3CC3)CC2)sc2c(NCc3ccco3)nc(Cl)nc12 ; N[C@H](Cc1sc2c(NCc3cccs3)nc(Cl)nc2c1Br)C1CC1 ; Cc1c(C[C@@H](N)C2CC2)sc2c(NCc3cccs3)nc(Cl)nc12]

wherein the form of the compound is selected from the group consisting of a salt, hydrate, solvate, and tautomer form thereof.

Another aspect of the method or use includes a compound of Formula (I) or a form thereof (wherein compound number (#¹) indicates that the salt form was isolated) includes a compound selected from the group consisting of:

| Cpd | Name |
|---|---|
| 1 | 2-chloro-N-[(pyridin-4-yl)methyl]thieno[3,2-d]pyrimidin-4-amine |
| 2 | 2-chloro-N-[(furan-2-yl)methyl]thieno[3,2-d]pyrimidin-4-amine |
| 3¹ | 6-[(2S)-2-aminopropyl]-2-chloro-N-[(furan-2-yl)methyl]thieno[3,2-d]pyrimidin-4-amine |
| 4¹ | 6-[(2S)-2-aminobutyl]-2-chloro-N-[(furan-2-yl)methyl]thieno[3,2-d]pyrimidin-4-amine |
| 5¹ | 6-[(2R)-2-amino-3-methylbutyl]-2-chloro-N-[(furan-2-yl)methyl]thieno[3,2-d]pyrimidin- |
| | 4-amine |
| 6¹ | 6-[(2R,3S)-2-amino-3-methylpentyl]-2-chloro-N-[(furan-2-yl)methyl]thieno[3,2- |
| | d]pyrimidin-4-amine |
| 7¹ | 6-[(2R)-2-amino-3,3-dimethylbutyl]-2-chloro-N-[(furan-2-yl)methyl]thieno[3,2- |
| | d]pyrimidin-4-amine |
| 8¹ | 6-[(2S)-2-aminopropyl]-N-[(furan-2-yl)methyl]thieno[3,2-d]pyrimidin-4-amine |
| 9¹ | 6-[(2S)-2-aminopropyl]-2-chloro-N-[(pyridin-4-yl)methyl]thieno[3,2-d]pyrimidin-4- |
| | amine |
| 10¹ | 6-[(2S)-2-aminobutyl]-2-chloro-N-[(thiophen-2-yl)methyl]thieno[3,2-d]pyrimidin-4- |
| | amine |
| 11¹ | 6-[(2S)-2-aminopropyl]-N-[(pyridin-4-yl)methyl]thieno[3,2-d]pyrimidin-4-amine |
| 12¹ | 6-[(2S)-2-aminopropyl]-2-chloro-N-[(furan-2-yl)methyl]-7-methylthieno[3,2- |
| | d]pyrimidin-4-amine |
| 13¹ | 6-[(2S)-2-aminobutyl]-2-chloro-N-[(furan-2-yl)methyl]-7-methylthieno[3,2-d]pyrimidin- |
| | 4-amine |
| 14¹ | 6-[(2S)-2-aminopropyl]-2-chloro-N-[(thiophen-2-yl)methyl]thieno[3,2-d]pyrimidin-4- |
| | amine |
| 15¹ | 6-[(2S)-2-aminopropyl]-N-[(furan-2-yl)methyl]-2,7-dimethylthieno[3,2-d]pyrimidin-4- |
| | amine |
| 16¹ | 6-[(2S)-2-aminopropyl]-2-ethyl-N-[(furan-2-yl)methyl]-7-methylthieno[3,2-d]pyrimidin- |
| | 4-amine |
| 17¹ | 6-[(2S)-2-aminopropyl]-2-cyclopropyl-N-[(furan-2-yl)methyl]-7-methylthieno[3,2- |
| | d]pyrimidin-4-amine |
| 18¹ | 2-chloro-N-[(furan-2-yl)methyl]-7-methylthieno[3,2-d]pyrimidin-4-amine |
| 19¹ | (2R)-2-amino-3-(2-chloro-4-{[(furan-2-yl)methyl]amino}-7-methylthieno[3,2- |
| | d]pyrimidin-6-yl)propan-1-ol |
| 20¹ | 6-[(2S)-2-aminopropyl]-4-{[(furan-2-yl)methyl]amino}-7-methylthieno[3,2- |
| | d]pyrimidine-2-carboxamide |
| 21¹ | 6-[(2S)-2-aminopropyl]-4-{[(furan-2-yl)methyl]amino}-7-methylthieno[3,2- |
| | d]pyrimidine-2-carbonitrile |
| 22 | (2R)-2-amino-3-(2-chloro-4-{[(furan-2-yl)methyl]amino}thieno[3,2-d]pyrimidin-6- |
| | yl)propan-1-ol |
| 23 | 2-chloro-7-methyl-N-[(pyridin-4-yl)methyl]thieno[3,2-d]pyrimidin-4-amine |
| 24¹ | 6-[(2S)-2-aminopropyl]-2-chloro-7-methyl-N-[(5-methylfuran-2-yl)methyl]thieno[3,2- |
| | d]pyrimidin-4-amine |
| 25 | N-[(furan-2-yl)methyl]-7-methyl-2-(trifluoromethyl)thieno[3,2-d]pyrimidin-4-amine |
| 26¹ | 6-[(2S)-2-aminopropyl]-N-[(furan-2-yl)methyl]-7-methyl-2-(trifluoromethyl)thieno[3,2- |
| | d]pyrimidin-4-amine |
| 27¹ | 6-[(2S)-2-aminopropyl]-2-chloro-7-methyl-N-[(4-methyl-1,3-thiazol-2- |
| | yl)methyl]thieno[3,2-d]pyrimidin-4-amine |
| 28¹ | 6-[(2S)-2-aminopropyl]-2-chloro-7-methyl-N-[(thiophen-2-yl)methyl]thieno[3,2- |
| | d]pyrimidin-4-amine |
| 29¹ | 6-[(2S)-2-aminopropyl]-2-chloro-7-methyl-N-[(1,3-thiazol-2-yl)methyl]thieno[3,2- |
| | d]pyrimidin-4-amine |
| 30¹ | 6-[(2S)-2-aminopropyl]-2-chloro-7-methyl-N-[(3-methylfuran-2-yl)methyl]thieno[3,2- |
| | d]pyrimidin-4-amine |
| 31¹ | 6-[(2S)-2-aminopropyl]-2-chloro-7-methyl-N-[(5-methyl-1,3-thiazol-2- |
| | yl)methyl]thieno[3,2-d]pyrimidin-4-amine |
| 32¹ | 6-[(2S)-2-aminopropyl]-2-chloro-7-methyl-N-[(pyrazin-2-yl)methyl]thieno[3,2- |
| | d]pyrimidin-4-amine |
| 33¹ | 6-[(2S)-2-aminopropyl]-2-chloro-N-[(5-fluorothiophen-2-yl)methyl]-7-methylthieno[3,2- |
| | d]pyrimidin-4-amine |
| 34¹ | 6-[(2S)-2-aminopropyl]-N-benzyl-2-chloro-7-methylthieno[3,2-d]pyrimidin-4-amine |
| 35¹ | 6-[(2S)-2-aminopropyl]-2-chloro-N-[(3-fluoropyridin-4-yl)methyl]-7-methylthieno[3,2- |
| | d]pyrimidin-4-amine |
| 36¹ | 6-[(2S)-2-aminopropyl]-2-chloro-7-cyclopropyl-N-[(furan-2-yl)methyl]thieno[3,2- |
| | d]pyrimidin-4-amine |
| 37¹ | 6-[(2S)-2-aminobutyl]-2-chloro-7-methyl-N-[(thiophen-2-yl)methyl]thieno[3,2- |
| | d]pyrimidin-4-amine |
| 38¹ | 6-[(2S)-2-aminopropyl]-2-bromo-N-[(furan-2-yl)methyl]-7-methylthieno[3,2- |
| | d]pyrimidin-4-amine |
| 39¹ | 6-[(2S)-2-aminopropyl]-2-chloro-7-methyl-N-[(1,2-oxazol-5-yl)methyl]thieno[3,2- |
| | d]pyrimidin-4-amine |
| 40¹ | 6-[(2S)-2-aminopropyl]-7-bromo-2-chloro-N-[(furan-2-yl)methyl]thieno[3,2-d]pyrimidin- |
| | 4-amine |
| 41¹ | 6-(azetidin-3-yl)-2-chloro-N-[(furan-2-yl)methyl]-7-methylthieno[3,2-d]pyrimidin-4- |
| | amine |
| 42 | 7-bromo-2-chloro-N-[(furan-2-yl)methyl]thieno[3,2-d]pyrimidin-4-amine |
| 43 | 7-bromo-2-chloro-N-[(pyridin-4-yl)methyl]thieno[3,2-d]pyrimidin-4-amine |
| 44¹ | 6-[(2S)-2-aminopropyl]-2-chloro-N-[(2-fluorophenyl)methyl]-7-methylthieno[3,2- |
| | d]pyrimidin-4-amine |
| 45¹ | 6-[(2S)-2-aminopropyl]-2-chloro-7-methyl-N-[(pyridin-4-yl)methyl]thieno[3,2- |
| | d]pyrimidin-4-amine |
| 46¹ | 6-[(1S)-1-aminoethyl]-7-bromo-2-chloro-N-[(furan-2-yl)methyl]thieno[3,2-d]pyrimidin- |
| | 4-amine |
| 47¹ | 6-[(1S)-1-aminoethyl]-2-chloro-N-[(furan-2-yl)methyl]-7-methylthieno[3,2-d]pyrimidin- |
| | 4-amine |
| 48¹ | 6-[(1S)-1-aminopropyl]-2-chloro-N-[(furan-2-yl)methyl]-7-methylthieno[3,2- |
| | d]pyrimidin-4-amine |
| 49¹ | 6-[(1R)-1-aminopropyl]-2-chloro-N-[(furan-2-yl)methyl]-7-methylthieno[3,2- |
| | d]pyrimidin-4-amine |
| 50¹ | 6-[(2S)-2-aminopropyl]-2-chloro-7-methyl-N-[(pyrimidin-4-yl)methyl]thieno[3,2- |
| | d]pyrimidin-4-amine |
| 51¹ | 6-[(2S)-2-amino-4-fluorobutyl]-2-chloro-N-[(furan-2-yl)methyl]-7-methylthieno[3,2- |
| | d]pyrimidin-4-amine |
| 52 | (4S)-4-[(2-chloro-4-{[(furan-2-yl)methyl]amino}-7-methylthieno[3,2-d]pyrimidin-6- |
| | yl)methyl]-1,3-oxazinan-2-one |
| 53¹ | 6-[(2S)-2-aminobutyl]-2-chloro-N-[(3-fluoropyridin-4-yl)methyl]-7-methylthieno[3,2- |
| | d]pyrimidin-4-amine |
| 54¹ | 6-[(2S)-2-aminobutyl]-2-chloro-7-methyl-N-[(1,3-thiazol-2-yl)methyl]thieno[3,2- |
| | d]pyrimidin-4-amine |
| 55¹ | 6-[(2S)-2-aminopropyl]-2-chloro-7-methyl-N-[(pyrimidin-2-yl)methyl]thieno[3,2- |
| | d]pyrimidin-4-amine |
| 56¹ | (2R)-2-amino-3-(2-chloro-7-methyl-4-{[(thiophen-2-yl)methyl]amino}thieno[3,2- |
| | d]pyrimidin-6-yl)propan-1-ol |
| 57¹ | 2-chloro-N-[(furan-2-yl)methyl]-7-methyl-6-(pyrrolidin-3-yl)thieno[3,2-d]pyrimidin-4- |
| | amine |
| 58¹ | 6-[(1S)-1-aminoethyl]-2-chloro-N-[(furan-2-yl)methyl]-7-phenylthieno[3,2-d]pyrimidin- |
| | 4-amine |
| 59¹ | 6-[(2S)-2-aminopropyl]-2-chloro-N-[(3-fluoropyridin-2-yl)methyl]-7-methylthieno[3,2- |
| | d]pyrimidin-4-amine |
| 60¹ | 6-[(2S)-2-aminopropyl]-2-chloro-N-[(2-fluoropyridin-3-yl)methyl]-7-methylthieno[3,2- |
| | d]pyrimidin-4-amine |
| 61¹ | 6-[(1S)-1-aminoethyl]-N-[(furan-2-yl)methyl]-2,7-diphenylthieno[3,2-d]pyrimidin-4- |
| | amine |
| 62¹ | 6-[(2S)-2-aminobutyl]-2-chloro-7-methyl-N-[(1,2-thiazol-5-yl)methyl]thieno[3,2- |
| | d]pyrimidin-4-amine |
| 63 | 3-(2-chloro-4-{[(furan-2-yl)methyl]amino}-7-methylthieno[3,2-d]pyrimidin-6-yl)propan- |
| | 1-ol |
| 64¹ | 6-[(2S)-2-aminopropyl]-2-chloro-N-[(3,5-difluoropyridin-4-yl)methyl]-7- |
| | methylthieno[3,2-d]pyrimidin-4-amine |
| 65¹ | 6-[(2S)-2-aminopropyl]-2-chloro-N-[(furan-2-yl)methyl]-7-(4-methoxyphenyl)thieno[3,2- |
| | d]pyrimidin-4-amine |
| 66 | (2S)-3-(2-chloro-4-{[(furan-2-yl)methyl]amino}-7-methylthieno[3,2-d]pyrimidin-6-yl)-2- |
| | methylpropan-1-ol |
| 67¹ | 6-(3-aminopropyl)-2-chloro-N-[(furan-2-yl)methyl]-7-methylthieno[3,2-d]pyrimidin-4- |
| | amine |
| 68¹ | 6-[(2S)-2-aminopropyl]-7-bromo-2-chloro-N-[(3-fluoropyridin-4-yl)methyl]thieno[3,2- |
| | d]pyrimidin-4-amine |
| 69¹ | 6-[(2S)-2-aminobutyl]-2-chloro-7-methyl-N-[(1,3-oxazol-2-yl)methyl]thieno[3,2- |
| | d]pyrimidin-4-amine |
| 70¹ | 6-[(2S)-3-amino-2-methylpropyl]-2-chloro-N-[(furan-2-yl)methyl]-7-methylthieno[3,2- |
| | d]pyrimidin-4-amine |
| 71 | (2R)-3-(2-chloro-4-{[(furan-2-yl)methyl]amino}-7-methylthieno[3,2-d]pyrimidin-6-yl)- |
| | 2-methylpropan-1-ol |
| 72¹ | 6-[(2R)-3-amino-2-methylpropyl]-2-chloro-N-[(furan-2-yl)methyl]-7-methylthieno[3,2- |
| | d]pyrimidin-4-amine |
| 73¹ | 6-[(2S)-2-aminopropyl]-2-chloro-N-[(1H-imidazol-2-yl)methyl]-7-methylthieno[3,2- |
| | d]pyrimidin-4-amine |
| 74¹ | 6-[(2S)-2-aminopropyl]-2-chloro-7-methyl-N-[(1,3-thiazol-5-yl)methyl]thieno[3,2- |
| | d]pyrimidin-4-amine |
| 75¹ | 6-[(2S)-2-aminopropyl]-2-chloro-7-methyl-N-[(1,3-oxazol-5-yl)methyl]thieno[3,2- |
| | d]pyrimidin-4-amine |
| 76¹ | 6-[(2R)-2-amino-3-methoxypropyl]-2-chloro-N-[(furan-2-yl)methyl]-7-methylthieno[3,2- |
| | d]pyrimidin-4-amine |
| 77¹ | 6-[(2S)-2-aminopropyl]-2-chloro-7-ethyl-N-[(furan-2-yl)methyl]thieno[3,2-d]pyrimidin- |
| | 4-amine |
| 78¹ | 2-chloro-6-[(2S)-2-(cyclobutylamino)propyl]-N-[(furan-2-yl)methyl]-7-methylthieno[3,2- |
| | d]pyrimidin-4-amine |
| 79¹ | 2-chloro-N-[(furan-2-yl)methyl]-7-methyl-6-[(2S)-2-(methylamino)propyl]thieno[3,2- |
| | d]pyrimidin-4-amine |
| 80 | 7-bromo-2-chloro-N-[(thiophen-2-yl)methyl]thieno[3,2-d]pyrimidin-4-amine |
| 81¹ | 6-[(2S)-2-aminopropyl]-2-chloro-7-methyl-N-[(1-methyl-1H-pyrazol-5- |
| | yl)methyl]thieno[3,2-d]pyrimidin-4-amine |
| 82¹ | 6-[(2S)-2-aminopropyl]-7-bromo-2-chloro-N-[(thiophen-2-yl)methyl]thieno[3,2- |
| | d]pyrimidin-4-amine |
| 83¹ | 6-[(2S)-2-aminopropyl]-7-bromo-4-{[(thiophen-2-yl)methyl]amino} thieno[3,2- |
| | d]pyrimidine-2-carbonitrile |
| 84 | 2-chloro-4-{[(thiophen-2-yl)methyl]amino}thieno[3,2-d]pyrimidine-7-carbonitrile |
| 85¹ | 6-[(2S)-2-aminopropyl]-4-{[(thiophen-2-yl)methyl]amino}thieno[3,2-d]pyrimidine-2,7- |
| | dicarbonitrile |
| 86¹ | 6-[(2S)-2-aminopropyl]-2-chloro-7-cyclopropyl-N-[(thiophen-2-yl)methyl]thieno[3,2- |
| | d]pyrimidin-4-amine |
| 87¹ | 6-[(2S)-2-aminopropyl]-2-chloro-7-phenyl-N-[(thiophen-2-yl)methyl]thieno[3,2- |
| | d]pyrimidin-4-amine |
| 88¹ | 6-[(2S)-2-aminopropyl]-2-chloro-7-(4-chlorophenyl)-N-[(thiophen-2- |
| | yl)methyl]thieno[3,2-d]pyrimidin-4-amine |
| 89¹ | 6-[(2S)-2-aminobutyl]-2-chloro-7-methyl-N-[(pyrimidin-4-yl)methyl]thieno[3,2- |
| | d]pyrimidin-4-amine |
| 90¹ | 6-[(2S)-2-aminopropyl]-2-chloro-N-[(3-fluorothiophen-2-yl)methyl]-7-methylthieno[3,2- |
| | d]pyrimidin-4-amine |
| 91¹ | 6-[(2S)-2-aminobutyl]-2-chloro-N-[(3-fluorothiophen-2-yl)methyl]-7-methylthieno[3,2- |
| | d]pyrimidin-4-amine |
| 92¹ | 6-[(2S)-2-aminopropyl]-2-chloro-N-[(4-fluoro-1,3-thiazol-2-yl)methyl]-7- |
| | methylthieno[3,2-d]pyrimidin-4-amine |
| 93¹ | 6-[(2S)-2-aminopropyl]-2-chloro-N-[(5-fluoro-1,3-thiazol-2-yl)methyl]-7- |
| | methylthieno[3,2-d]pyrimidin-4-amine |
| 94¹ | 6-[(1R)-1-aminoethyl]-2-chloro-N-[(furan-2-yl)methyl]-7-methylthieno[3,2-d]pyrimidin- |
| | 4-amine |
| 95 | 2-chloro-N-[(pyrimidin-4-yl)methyl]thieno[3,2-d]pyrimidin-4-amine |
| 96 | 2-chloro-N-[(1,3-thiazol-2-yl)methyl]thieno[3,2-d]pyrimidin-4-amine |
| 97¹ | (3S)-3-amino-4-(2-chloro-4-{[(furan-2-yl)methyl]amino}-7-methylthieno[3,2- |
| | d]pyrimidin-6-yl)butan-1-ol |
| 98¹ | 6-[(2S)-2-aminopropyl]-2-chloro-N-[(5-fluoropyrimidin-4-yl)methyl]-7- |
| | methylthieno[3,2-d]pyrimidin-4-amine |
| 99¹ | (2R)-2-amino-3-(2-chloro-7-methoxy-4-{[(thiophen-2-yl)methyl]amino}thieno[3,2- |
| | d]pyrimidin-6-yl)propan-1-ol |
| 100 | 6-[(2S)-2-aminopropyl]-2-chloro-7-methyl-N-[(thiophen-3-yl)methyl]thieno[3,2- |
| | d]pyrimidin-4-amine |
| 101 | 6-[(2S)-2-aminopropyl]-2-chloro-7-methyl-N-[(1H-pyrazol-5-yl)methyl]thieno[3,2- |
| | d]pyrimidin-4-amine |
| 102 | 6-[(2S)-2-aminopropyl]-2-chloro-7-methyl-N-[(1-methyl-1H-imidazol-2- |
| | yl)methyl]thieno[3,2-d]pyrimidin-4-amine |
| 103 | 6-[(2S)-2-aminopropyl]-2-chloro-N-[(furan-3-yl)methyl]-7-methylthieno[3,2- |
| | d]pyrimidin-4-amine |
| 104 | 6-[(2S)-2-aminopropyl]-2-chloro-7-methyl-N-[(1H-pyrrol-2-yl)methyl]thieno[3,2- |
| | d]pyrimidin-4-amine |
| 105 | 6-[(2S)-2-aminopropyl]-2-chloro-7-methyl-N-[(1,2-oxazol-3-yl)methyl]thieno[3,2- |
| | d]pyrimidin-4-amine |
| 106 | 6-[(2S)-2-aminopropyl]-2-chloro-7-methyl-N-[(1-methyl-1H-pyrazol-3- |
| | yl)methyl]thieno[3,2-d]pyrimidin-4-amine |
| 107 | 6-[(2S)-2-aminopropyl]-2-chloro-7-methyl-N-[(1,2-oxazol-4-yl)methyl]thieno[3,2- |
| | d]pyrimidin-4-amine |
| 108 | 6-[(2S)-2-aminopropyl]-2-chloro-7-methyl-N-[(1,2-thiazol-4-yl)methyl]thieno[3,2- |
| | d]pyrimidin-4-amine |
| 109 | 6-[(2S)-2-aminopropyl]-2-chloro-7-methyl-N-[(1H-pyrazol-4-yl)methyl]thieno[3,2- |
| | d]pyrimidin-4-amine |
| 110 | 6-[(2S)-2-aminopropyl]-2-chloro-7-methyl-N-[(1-methyl-1H-pyrazol-4- |
| | yl)methyl]thieno[3,2-d]pyrimidin-4-amine |
| 111 | 6-[(2S)-2-aminopropyl]-2-chloro-7-methyl-N-[(1,3-oxazol-2-yl)methyl]thieno[3,2- |
| | d]pyrimidin-4-amine |
| 112 | 6-[(2S)-2-aminopropyl]-2-chloro-7-methyl-N-[(1,3-oxazol-4-yl)methyl]thieno[3,2- |
| | d]pyrimidin-4-amine |
| 113 | 6-[(2S)-2-aminopropyl]-2-chloro-7-methyl-N-[(1,3-thiazol-4-yl)methyl]thieno[3,2- |
| | d]pyrimidin-4-amine |
| 114 | 6-[(2S)-2-aminopropyl]-2-chloro-N-[(1H-imidazol-5-yl)methyl]-7-methylthieno[3,2- |
| | d]pyrimidin-4-amine |
| 115 | 6-[(2S)-2-aminopropyl]-2-chloro-7-methyl-N-[(1-methyl-1H-imidazol-5- |
| | yl)methyl]thieno[3,2-d]pyrimidin-4-amine |
| 116 | 6-[(2S)-2-aminopropyl]-2-chloro-7-methyl-N-[(1-methyl-1H-imidazol-4- |
| | yl)methyl]thieno[3,2-d]pyrimidin-4-amine |
| 117 | 6-[(2S)-2-aminopropyl]-2-chloro-7-methyl-N-[(2H-1,2,3-triazol-4-yl)methyl]thieno[3,2- |
| | d]pyrimidin-4-amine |
| 118 | 6-[(2S)-2-aminopropyl]-2-chloro-7-methyl-N-[(1H-tetrazol-5-yl)methyl]thieno[3,2- |
| | d]pyrimidin-4-amine |
| 119 | 6-[(2S)-2-aminopropyl]-2-chloro-7-methyl-N-[(1-methyl-1H-pyrrol-2- |
| | yl)methyl]thieno[3,2-d]pyrimidin-4-amine |
| 120¹ | 6-[(2S)-2-aminopropyl]-2-chloro-7-methyl-N-[(1H-pyrrol-3-yl)methyl]thieno[3,2- |
| | d]pyrimidin-4-amine |
| 121¹ | 6-[(S)-(1-(1-aminoethyl)cyclopropyl)]-2-chloro-N-[(furan-2-yl)methyl]-7- |
| | methylthieno[3,2-d]pyrimidin-4-amine |
| 122¹ | 6-[(S)-(1-(amino(cyclopropyl)methyl)cyclopropyl)]-2-chloro-N-[(furan-2-yl)methyl]-7- |
| | methylthieno[3,2-d]pyrimidin-4-amine |
| 123¹ | 6-[(2R)-2-amino-2-cyclopropylethyl]-7-bromo-2-chloro-N-[(thiophen-2- |
| | yl)methyl]thieno[3,2-d]pyrimidin-4-amine, and |
| 124¹ | 6-[(2R)-2-amino-2-cyclopropylethyl]-2-chloro-7-methyl-N-[(thiophen-2- |
| | yl)methyl]thieno[3,2-d]pyrimidin-4-amine; |

wherein the form of the compound is selected from the group consisting of a salt, hydrate, solvate, and tautomer form thereof.

Another aspect of the method or use includes a compound of Formula (I) or a form thereof is a compound salt selected from the group consisting of:

| Cpd | Name |
|---|---|
| 3 | 6-[(2S)-2-aminopropyl]-2-chloro-N-[(furan-2-yl)methyl]thieno[3,2-d]pyrimidin-4-amine |
| | dihydrochloride |
| 4 | 6-[(2S)-2-aminobutyl]-2-chloro-N-[(furan-2-yl)methyl]thieno[3,2-d]pyrimidin-4-amine |
| | dihydrochloride |
| 5 | 6-[(2R)-2-amino-3-methylbutyl]-2-chloro-N-[(furan-2-yl)methyl]thieno[3,2- |
| | d]pyrimidin-4-amine dihydrochloride |
| 6 | 6-[(2R,3S)-2-amino-3-methylpentyl]-2-chloro-N-[(furan-2-yl)methyl]thieno[3,2- |
| | d]pyrimidin-4-amine dihydrochloride |
| 7 | 6-[(2R)-2-amino-3,3-dimethylbutyl]-2-chloro-N-[(furan-2-yl)methyl]thieno[3,2- |
| | d]pyrimidin-4-amine dihydrochloride |
| 8 | 6-[(2S)-2-aminopropyl]-N-[(furan-2-yl)methyl]thieno[3,2-d]pyrimidin-4-amine |
| | dihydrochloride |
| 9 | 6-[(2S)-2-aminopropyl]-2-chloro-N-[(pyridin-4-yl)methyl]thieno[3,2-d]pyrimidin-4- |
| | amine dihydrochloride |
| 10 | 6-[(2S)-2-aminobutyl]-2-chloro-N-[(thiophen-2-yl)methyl]thieno[3,2-d]pyrimidin-4- |
| | amine dihydrochloride |
| 11 | 6-[(2S)-2-aminopropyl]-N-[(pyridin-4-yl)methyl]thieno[3,2-d]pyrimidin-4-amine |
| | dihydrochloride |
| 12 | 6-[(2S)-2-aminopropyl]-2-chloro-N-[(furan-2-yl)methyl]-7-methylthieno[3,2- |
| | d]pyrimidin-4-amine dihydrochloride |
| 13 | 6-[(2S)-2-aminobutyl]-2-chloro-N-[(furan-2-yl)methyl]-7-methylthieno[3,2- |
| | d]pyrimidin-4-amine dihydrochloride |
| 14 | 6-[(2S)-2-aminopropyl]-2-chloro-N-[(thiophen-2-yl)methyl]thieno[3,2-d]pyrimidin-4- |
| | amine dihydrochloride |
| 15 | 6-[(2S)-2-aminopropyl]-N-[(furan-2-yl)methyl]-2,7-dimethylthieno[3,2-d]pyrimidin-4- |
| | amine dihydrochloride |
| 16 | 6-[(2S)-2-aminopropyl]-2-ethyl-N-[(furan-2-yl)methyl]-7-methylthieno[3,2- |
| | d]pyrimidin-4-amine dihydrochloride |
| 17 | 6-[(2S)-2-aminopropyl]-2-cyclopropyl-N-[(furan-2-yl)methyl]-7-methylthieno[3,2- |
| | d]pyrimidin-4-amine dihydrochloride |
| 18 | 2-chloro-N-[(furan-2-yl)methyl]-7-methylthieno[3,2-d]pyrimidin-4-amine |
| | hydrochloride |
| 19 | (2R)-2-amino-3-(2-chloro-4-{[(furan-2-yl)methyl]amino}-7-methylthieno[3,2- |
| | d]pyrimidin-6-yl)propan-1-ol dihydrochloride |
| 20 | 6-[(2S)-2-aminopropyl]-4-{[(furan-2-yl)methyl]amino}-7-methylthieno[3,2- |
| | d]pyrimidine-2-carboxamide trifluoroacetate |
| 21 | 6-[(2S)-2-aminopropyl]-4-{[(furan-2-yl)methyl]amino}-7-methylthieno[3,2- |
| | d]pyrimidine-2-carbonitrile trifluoroacetate |
| 24 | 6-[(2S)-2-aminopropyl]-2-chloro-7-methyl-N-[(5-methylfuran-2-yl)methyl]thieno[3,2- |
| | d]pyrimidin-4-amine dihydrochloride |
| 26 | 6-[(2S)-2-aminopropyl]-N-[(furan-2-yl)methyl]-7-methyl-2-(trifluoromethyl)thieno[3,2- |
| | d]pyrimidin-4-amine dihydrochloride |
| 27 | 6-[(2S)-2-aminopropyl]-2-chloro-7-methyl-N-[(4-methyl-1,3-thiazol-2- |
| | yl)methyl]thieno[3,2-d]pyrimidin-4-amine dihydrochloride |
| 28 | 6-[(2S)-2-aminopropyl]-2-chloro-7-methyl-N-[(thiophen-2-yl)methyl]thieno[3,2- |
| | d]pyrimidin-4-amine dihydrochloride |
| 29 | 6-[(2S)-2-aminopropyl]-2-chloro-7-methyl-N-[(1,3-thiazol-2-yl)methyl]thieno[3,2- |
| | d]pyrimidin-4-amine trifluoroacetate |
| 30 | 6-[(2S)-2-aminopropyl]-2-chloro-7-methyl-N-[(3-methylfuran-2-yl)methyl]thieno[3,2- |
| | d]pyrimidin-4-amine dihydrochloride |
| 31 | 6-[(2S)-2-aminopropyl]-2-chloro-7-methyl-N-[(5-methyl-1,3-thiazol-2- |
| | yl)methyl]thieno[3,2-d]pyrimidin-4-amine dihydrochloride |
| 32 | 6-[(2S)-2-aminopropyl]-2-chloro-7-methyl-N-[(pyrazin-2-yl)methyl]thieno[3,2- |
| | d]pyrimidin-4-amine dihydrochloride |
| 33 | 6-[(2S)-2-aminopropyl]-2-chloro-N-[(5-fluorothiophen-2-yl)methyl]-7- |
| | methylthieno[3,2-d]pyrimidin-4-amine dihydrochloride |
| 34 | 6-[(2S)-2-aminopropyl]-N-benzyl-2-chloro-7-methylthieno[3,2-d]pyrimidin-4-amine |
| | dihydrochloride |
| 35 | 6-[(2S)-2-aminopropyl]-2-chloro-N-[(3-fluoropyridin-4-yl)methyl]-7-methylthieno[3,2- |
| | d]pyrimidin-4-amine dihydrochloride |
| 36 | 6-[(2S)-2-aminopropyl]-2-chloro-7-cyclopropyl-N-[(furan-2-yl)methyl]thieno[3,2- |
| | d]pyrimidin-4-amine trifluoroacetate |
| 37 | 6-[(2S)-2-aminobutyl]-2-chloro-7-methyl-N-[(thiophen-2-yl)methyl]thieno[3,2- |
| | d]pyrimidin-4-amine dihydrochloride |
| 38 | 6-[(2S)-2-aminopropyl]-2-bromo-N-[(furan-2-yl)methyl]-7-methylthieno[3,2- |
| | d]pyrimidin-4-amine trifluoroacetate |
| 39 | 6-[(2S)-2-aminopropyl]-2-chloro-7-methyl-N-[(1,2-oxazol-5-yl)methyl]thieno[3,2- |
| | d]pyrimidin-4-amine hydrochloride |
| 40 | 6-[(2S)-2-aminopropyl]-7-bromo-2-chloro-N-[(furan-2-yl)methyl]thieno[3,2- |
| | d]pyrimidin-4-amine dihydrochloride |
| 41 | 6-(azetidin-3-yl)-2-chloro-N-[(furan-2-yl)methyl]-7-methylthieno[3,2-d]pyrimidin-4- |
| | amine trifluoroacetate |
| 44 | 6-[(2S)-2-aminopropyl]-2-chloro-N-[(2-fluorophenyl)methyl]-7-methylthieno[3,2- |
| | d]pyrimidin-4-amine dihydrochloride |
| 45 | 6-[(2S)-2-aminopropyl]-2-chloro-7-methyl-N-[(pyridin-4-yl)methyl]thieno[3,2- |
| | d]pyrimidin-4-amine dihydrochloride |
| 46 | 6-[(1S)-1-aminoethyl]-7-bromo-2-chloro-N-[(furan-2-yl)methyl]thieno[3,2-d]pyrimidin- |
| | 4-amine hydrochloride |
| 47 | 6-[(1S)-1-aminoethyl]-2-chloro-N-[(furan-2-yl)methyl]-7-methylthieno[3,2- |
| | d]pyrimidin-4-amine hydrochloride |
| 48 | 6-[(1S)-1-aminopropyl]-2-chloro-N-[(furan-2-yl)methyl]-7-methylthieno[3,2- |
| | d]pyrimidin-4-amine hydrochloride |
| 49 | 6-[(1R)-1-aminopropyl]-2-chloro-N-[(furan-2-yl)methyl]-7-methylthieno[3,2- |
| | d]pyrimidin-4-amine hydrochloride |
| 50 | 6-[(2S)-2-aminopropyl]-2-chloro-7-methyl-N-[(pyrimidin-4-yl)methyl]thieno[3,2- |
| | d]pyrimidin-4-amine dihydrochloride |
| 51 | 6-[(2S)-2-amino-4-fluorobutyl]-2-chloro-N-[(furan-2-yl)methyl]-7-methylthieno[3,2- |
| | d]pyrimidin-4-amine dihydrochloride |
| 53 | 6-[(2S)-2-aminobutyl]-2-chloro-N-[(3-fluoropyridin-4-yl)methyl]-7-methylthieno[3,2- |
| | d]pyrimidin-4-amine dihydrochloride |
| 54 | 6-[(2S)-2-aminobutyl]-2-chloro-7-methyl-N-[(1,3-thiazol-2-yl)methyl]thieno[3,2- |
| | d]pyrimidin-4-amine dihydrochloride |
| 55 | 6-[(2S)-2-aminopropyl]-2-chloro-7-methyl-N-[(pyrimidin-2-yl)methyl]thieno[3,2- |
| | d]pyrimidin-4-amine dihydrochloride |
| 56 | (2R)-2-amino-3-(2-chloro-7-methyl-4-{[(thiophen-2-yl)methyl]amino}thieno[3,2- |
| | d]pyrimidin-6-yl)propan-1-ol dihydrochloride |
| 57 | 2-chloro-N-[(furan-2-yl)methyl]-7-methyl-6-(pyrrolidin-3-yl)thieno[3,2-d]pyrimidin-4- |
| | amine formate |
| 58 | 6-[(1S)-1-aminoethyl]-2-chloro-N-[(furan-2-yl)methyl]-7-phenylthieno[3,2-d]pyrimidin- |
| | 4-amine formate |
| 59 | 6-[(2S)-2-aminopropyl]-2-chloro-N-[(3-fluoropyridin-2-yl)methyl]-7-methylthieno[3,2- |
| | d]pyrimidin-4-amine dihydrochloride |
| 60 | 6-[(2S)-2-aminopropyl]-2-chloro-N-[(2-fluoropyridin-3-yl)methyl]-7-methylthieno[3,2- |
| | d]pyrimidin-4-amine dihydrochloride |
| 61 | 6-[(1S)-1-aminoethyl]-N-[(furan-2-yl)methyl]-2,7-diphenylthieno[3,2-d]pyrimidin-4- |
| | amine formate |
| 62 | 6-[(2S)-2-aminobutyl]-2-chloro-7-methyl-N-[(1,2-thiazol-5-yl)methyl]thieno[3,2- |
| | d]pyrimidin-4-amine formate |
| 64 | 6-[(2S)-2-aminopropyl]-2-chloro-N-[(3,5-difluoropyridin-4-yl)methyl]-7- |
| | methylthieno[3,2-d]pyrimidin-4-amine dihydrochloride |
| 65 | 6-[(2S)-2-aminopropyl]-2-chloro-N-[(furan-2-yl)methyl]-7-(4- |
| | methoxyphenyl)thieno[3,2-d]pyrimidin-4-amine formate |
| 67 | 6-(3-aminopropyl)-2-chloro-N-[(furan-2-yl)methyl]-7-methylthieno[3,2-d]pyrimidin-4- |
| | amine hydrochloride |
| 68 | 6-[(2S)-2-aminopropyl]-7-bromo-2-chloro-N-[(3-fluoropyridin-4-yl)methyl]thieno[3,2- |
| | d]pyrimidin-4-amine dihydrochloride |
| 69 | 6-[(2S)-2-aminobutyl]-2-chloro-7-methyl-N-[(1,3-oxazol-2-yl)methyl]thieno[3,2- |
| | d]pyrimidin-4-amine dihydrochloride |
| 70 | 6-[(2S)-3-amino-2-methylpropyl]-2-chloro-N-[(furan-2-yl)methyl]-7-methylthieno[3,2- |
| | d]pyrimidin-4-amine hydrochloride |
| 72 | 6-[(2R)-3-amino-2-methylpropyl]-2-chloro-N-[(furan-2-yl)methyl]-7-methylthieno[3,2- |
| | d]pyrimidin-4-amine hydrochloride |
| 73 | 6-[(2S)-2-aminopropyl]-2-chloro-N-[(1H-imidazol-2-yl)methyl]-7-methylthieno[3,2- |
| | d]pyrimidin-4-amine dihydrochloride |
| 74 | 6-[(2S)-2-aminopropyl]-2-chloro-7-methyl-N-[(1,3-thiazol-5-yl)methyl]thieno[3,2- |
| | d]pyrimidin-4-amine dihydrochloride |
| 75 | 6-[(2S)-2-aminopropyl]-2-chloro-7-methyl-N-[(1,3-oxazol-5-yl)methyl]thieno[3,2- |
| | d]pyrimidin-4-amine dihydrochloride |
| 76 | 6-[(2R)-2-amino-3-methoxypropyl]-2-chloro-N-[(furan-2-yl)methyl]-7- |
| | methylthieno[3,2-d]pyrimidin-4-amine dihydrochloride |
| 77 | 6-[(2S)-2-aminopropyl]-2-chloro-7-ethyl-N-[(furan-2-yl)methyl]thieno[3,2-d]pyrimidin- |
| | 4-amine hydrochloride |
| 78 | 2-chloro-6-[(2S)-2-(cyclobutylamino)propyl]-N-[(furan-2-yl)methyl]-7- |
| | methylthieno[3,2-d]pyrimidin-4-amine formate |
| 79 | 2-chloro-N-[(furan-2-yl)methyl]-7-methyl-6-[(2S)-2-(methylamino)propyl]thieno[3,2- |
| | d]pyrimidin-4-amine hydrochloride |
| 81 | 6-[(2S)-2-aminopropyl]-2-chloro-7-methyl-N-[(1-methyl-1H-pyrazol-5- |
| | yl)methyl]thieno[3,2-d]pyrimidin-4-amine hydrochloride |
| 82 | 6-[(2S)-2-aminopropyl]-7-bromo-2-chloro-N-[(thiophen-2-yl)methyl]thieno[3,2- |
| | d]pyrimidin-4-amine hydrochloride |
| 83 | 6-[(2S)-2-aminopropyl]-7-bromo-4-{[(thiophen-2-yl)methyl]amino}thieno[3,2- |
| | d]pyrimidine-2-carbonitrile formate |
| 85 | 6-[(2S)-2-aminopropyl]-4-{[(thiophen-2-yl)methyl]amino}thieno[3,2-d]pyrimidine-2,7- |
| | dicarbonitrile hydrochloride |
| 86 | 6-[(2S)-2-aminopropyl]-2-chloro-7-cyclopropyl-N-[(thiophen-2-yl)methyl]thieno[3,2- |
| | d]pyrimidin-4-amine formate |
| 87 | 6-[(2S)-2-aminopropyl]-2-chloro-7-phenyl-N-[(thiophen-2-yl)methyl]thieno[3,2- |
| | d]pyrimidin-4-amine hydrochloride |
| 88 | 6-[(2S)-2-aminopropyl]-2-chloro-7-(4-chlorophenyl)-N-[(thiophen-2- |
| | yl)methyl]thieno[3,2-d]pyrimidin-4-amine hydrochloride |
| 89 | 6-[(2S)-2-aminobutyl]-2-chloro-7-methyl-N-[(pyrimidin-4-yl)methyl]thieno[3,2- |
| | d]pyrimidin-4-amine dihydrochloride |
| 90 | 6-[(2S)-2-aminopropyl]-2-chloro-N-[(3-fluorothiophen-2-yl)methyl]-7- |
| | methylthieno[3,2-d]pyrimidin-4-amine dihydrochloride |
| 91 | 6-[(2S)-2-aminobutyl]-2-chloro-N-[(3-fluorothiophen-2-yl)methyl]-7-methylthieno[3,2- |
| | d]pyrimidin-4-amine dihydrochloride |
| 92 | 6-[(2S)-2-aminopropyl]-2-chloro-N-[(4-fluoro-1,3-thiazol-2-yl)methyl]-7- |
| | methylthieno[3,2-d]pyrimidin-4-amine dihydrochloride |
| 93 | 6-[(2S)-2-aminopropyl]-2-chloro-N-[(5-fluoro-1,3-thiazol-2-yl)methyl]-7- |
| | methylthieno[3,2-d]pyrimidin-4-amine dihydrochloride |
| 94 | 6-[(1R)-1-aminoethyl]-2-chloro-N-[(furan-2-yl)methyl]-7-methylthieno[3,2- |
| | d]pyrimidin-4-amine hydrochloride |
| 97 | (3S)-3-amino-4-(2-chloro-4-{[(furan-2-yl)methyl]amino}-7-methylthieno[3,2- |
| | d]pyrimidin-6-yl)butan-1-ol dihydrochloride |
| 98 | 6-[(2S)-2-aminopropyl]-2-chloro-N-[(5-fluoropyrimidin-4-yl)methyl]-7- |
| | methylthieno[3,2-d]pyrimidin-4-amine formate |
| 99 | (2R)-2-amino-3-(2-chloro-7-methoxy-4-{[(thiophen-2-yl)methyl]amino}thieno[3,2- |
| | d]pyrimidin-6-yl)propan-1-ol dihydrochloride |
| 121 | 6-[(S)-(1-(1-aminoethyl)cyclopropyl)]-2-chloro-N-[(furan-2-yl)methyl]-7- |
| | methylthieno[3,2-d]pyrimidin-4-amine formate |
| 122 | 6-[(S)-(1-(amino(cyclopropyl)methyl)cyclopropyl)]-2-chloro-N-[(furan-2-yl)methyl]-7- |
| | methylthieno[3,2-d]pyrimidin-4-amine formate |
| 123 | 6-[(2R)-2-amino-2-cyclopropylethyl]-7-bromo-2-chloro-N-[(thiophen-2- |
| | yl)methyl]thieno[3,2-d]pyrimidin-4-amine dihydrochloride, and |
| 124 | 6-[(2R)-2-amino-2-cyclopropylethyl]-2-chloro-7-methyl-N-[(thiophen-2- |
| | yl)methyl]thieno[3,2-d]pyrimidin-4-amine dihydrochloride; |

wherein the form of the compound is selected from the group consisting of hydrate, solvate, and tautomer form thereof.

One aspect of the method or use includes a compound of Formula (I) or a form thereof for the treatment of a neurodegenerative disease in a subject characterized by the accumulation and aggregation of aberrant forms of MAPT.

One aspect of the method or use includes a compound of Formula (I) or a form thereof for the treatment of a neurodegenerative disease in a subject characterized by the formation of neurofibrillary tangles and paired helical filaments in neurons and glia of the affected brain regions.

One aspect of the method or use includes the compound of Formula (I) or a form thereof, wherein the neurogenerative disease is selected from the group consisting of Alzheimer's disease, dementia pugilistica, Guam Amyotrophic lateral sclerosis-Parkinsonism-Dementia (Guam ALS/PD), Pick Disease, Argyrophilic grain dementia, Nieman-Pick type C, Subacute sclerosing panencephalitis (SSPE), Progressive supranuclear palsy (PSP), multisystem atrophy (MSA), Corticobasoganlionic degeneration, Frontotemporal dementia with parkinsonism-17 (FTDP-17), Postencephalitic Parkinsonism (PEP), Autosomal recessive Parkinsonism, frontotemporal dementia, and progressive supranuclear palsy.

One aspect includes a method for inducing exon 10 skipping in the MAPT pre-mRNA, comprising contacting a human cell with a compound of Formula (I) or a form thereof.

One aspect includes a method for producing MAPT ΔE4 mRNA, comprising contacting a human cell with a compound of Formula (I) or a form thereof.

One aspect includes a method for lowering MAPT4R protein, comprising contacting a human cell with a compound of Formula (I) or a form thereof.

One aspect of the present description relates to a pharmaceutical composition comprising a compound of Formula (I) or a form thereof and at least one pharmaceutically acceptable excipient for administering to a subject for the treatment of a neurodegenerative disease characterized by the accumulation and aggregation of aberrant forms of MAPT.

One aspect of the present description relates to the manufacture of a medicament for the treatment of a neurodegenerative disease characterized by the accumulation and aggregation of aberrant forms of MAPT, in a subject comprising a compound of Formula (I) or a form thereof and at least one pharmaceutically acceptable excipient.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used.

The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Chemical Definitions

The chemical terms used above and throughout the description herein, unless specifically defined otherwise, shall be understood by one of ordinary skill in the art to have the following indicated meanings.

As used herein, the term “C₁₋₆alkyl” generally refers to saturated hydrocarbon radicals having from one to eight carbon atoms in a straight or branched chain configuration, including, but not limited to, methyl, ethyl, n-propyl (also referred to as propyl or propanyl), isopropyl, n-butyl (also referred to as butyl or butanyl), isobutyl, sec-butyl, tert-butyl, n-pentyl (also referred to as pentyl or pentanyl), n-hexyl (also referred to as hexyl or hexanyl), and the like. In certain aspects, C₁₋₆alkyl includes, but is not limited to, C₁₋₆alkyl, C₁₋₄alkyl and the like. A C₁₋₆alkyl radical is optionally substituted with substituent species as described herein where allowed by available valences.

As used herein, the term “hetero-C₁₋₆alkyl” generally refers to saturated hydrocarbon radicals having from one to six carbon atoms in a straight or branched chain configuration, in which one or more heteroatoms, such as an O, S or N atom, are members in the chain, including, but not limited to, but not limited to, hetero-methyl, hetero-ethyl, hetero-propyl, hetero-butyl, hetero-pentyl, hetero-hexyl and the like. In certain aspects, hetero-C₁₋₆alkyl includes, but is not limited to, hetero-C₂₋₆alkyl, hetero-C₁₋₄alkyl, hetero-C₂₋₄alkyl and the like. A hetero-C₁₋₆alkyl radical is optionally substituted with substituent species as described herein where allowed by available valences.

As used herein, the term “C₂₋₆alkenyl” generally refers to partially unsaturated hydrocarbon radicals having from two to eight carbon atoms in a straight or branched chain configuration and one or more carbon-carbon double bonds therein, including, but not limited to, ethenyl (also referred to as vinyl), allyl, propenyl and the like. In certain aspects, C₂₋₆alkenyl includes, but is not limited to, C₂₋₆alkenyl, C₂₋₄alkenyl and the like. A C₂₋₆alkenyl radical is optionally substituted with substituent species as described herein where allowed by available valences.

As used herein, the term “C₂₋₆alkynyl” generally refers to partially unsaturated hydrocarbon radicals having from two to eight carbon atoms in a straight or branched chain configuration and one or more carbon-carbon triple bonds therein, including, but not limited to, ethynyl (also referred to as acetylenyl), propynyl, butynyl and the like. In certain aspects, C₂₋₆alkynyl includes, but is not limited to, C₂₋₆alkynyl, C₂₋₄alkynyl and the like. A C₂₋₆alkynyl radical is optionally substituted with substituent species as described herein where allowed by available valences.

As used herein, the term “C₁₋₆alkoxy” generally refers to saturated hydrocarbon radicals having from one to eight carbon atoms in a straight or branched chain configuration of the formula: —O—C₁₋₆alkyl, including, but not limited to, methoxy, ethoxy, n-propoxy, isopropoxy, n-butoxy, isobutoxy, sec-butoxy, tert-butoxy, n-pentoxy, n-hexoxy and the like. In certain aspects, C₁₋₆alkoxy includes, but is not limited to, C₁₋₆alkoxy, C₁₋₄alkoxy and the like. A C₁₋₆alkoxy radical is optionally substituted with substituent species as described herein where allowed by available valences.

As used herein, the term “oxo” refers to a radical of the formula: ═O.

As used herein, the term “carboxyl” refers to a radical of the formula: —COOH, —C(O)OH or —CO₂H.

As used herein, the term “carbamoyl” refers to a radical of the formula: —C(O)NH₂.

As used herein, the term “C₃₋₁₀cycloalkyl” generally refers to a saturated or partially unsaturated monocyclic, bicyclic or polycyclic hydrocarbon radical, including, but not limited to, cyclopropyl, cyclobutyl, cyclopentyl, cyclohexyl, cyclohexenyl, cycloheptyl, cyclooctyl, 1H-indanyl, indenyl, tetrahydro-naphthalenyl and the like. In certain aspects, C₃₋₁₀cycloalkyl includes, but is not limited to, C₃₋₈cycloalkyl, C₅₋₈cycloalkyl, C₃₋₁₀cycloalkyl and the like. A C₃₋₁₀cycloalkyl radical is optionally substituted with substituent species as described herein where allowed by available valences.

As used herein, the term “aryl” generally refers to a monocyclic, bicyclic or polycyclic aromatic carbon atom ring structure radical, including, but not limited to, phenyl, naphthyl, anthracenyl, fluorenyl, azulenyl, phenanthrenyl and the like. An aryl radical is optionally substituted with substituent species as described herein where allowed by available valences.

As used herein, the term “heteroaryl” generally refers to a monocyclic, bicyclic or polycyclic aromatic carbon atom ring structure radical in which one or more carbon atom ring members have been replaced, where allowed by structural stability, with one or more heteroatoms, such as an O, S or N atom, including, but not limited to, furanyl, thiophenyl, pyrrolyl, pyrazolyl, imidazolyl, isoxazolyl, isothiazolyl, oxazolyl, 1,3-thiazolyl, triazolyl, oxadiazolyl, thiadiazolyl, tetrazolyl, pyridinyl, pyrimidinyl, pyrazinyl, pyridazinyl, triazinyl, indolyl, indazolyl, indolizinyl, isoindolyl, benzofuranyl, benzothiophenyl, benzoimidazolyl, 1,3-benzothiazolyl, 1,3-benzoxazolyl, purinyl, quinolinyl, isoquinolinyl, quinazolinyl, quinoxalinyl and the like. A heteroaryl radical is optionally substituted on a carbon or nitrogen atom ring member with substituent species as described herein where allowed by available valences.

In certain aspects, the nomenclature for a heteroaryl radical may differ, such as in non-limiting examples where furanyl may also be referred to as furyl, thiophenyl may also be referred to as thienyl, pyridinyl may also be referred to as pyridyl, benzothiphenyl may also be referred to as benzothienyl and 1,3-benzoxazolyl may also be referred to as 1,3-benzooxazolyl.

In certain other aspects, the term for a heteroaryl radical may also include other regioisomers, such as in non-limiting examples where the term pyrrolyl may also include 2H-pyrrolyl, 3H-pyrrolyl and the like, the term pyrazolyl may also include 1H-pyrazolyl and the like, the term imidazolyl may also include 1H-imidazolyl and the like, the term triazolyl may also include 1H-1,2,3-triazolyl and the like, the term oxadiazolyl may also include 1,2,4-oxadiazolyl, 1,3,4-oxadiazolyl and the like, the term tetrazolyl may also include 1H-tetrazolyl, 2H-tetrazolyl and the like, the term indolyl may also include 1H-indolyl and the like, the term indazolyl may also include 1H-indazolyl, 2H-indazolyl and the like, the term benzoimidazolyl may also include 1H-benzoimidazolyl and the term purinyl may also include 9H-purinyl and the like.

As used herein, the term “heterocyclyl” generally refers to a saturated or partially unsaturated monocyclic, bicyclic or polycyclic carbon atom ring structure radical in which one or more carbon atom ring members have been replaced, where allowed by structural stability, with a heteroatom, such as an O, S or N atom, including, but not limited to, oxiranyl, oxetanyl, azetidinyl, tetrahydrofuranyl, pyrrolinyl, pyrrolidinyl, pyrazolinyl, pyrazolidinyl, imidazolinyl, imidazolidinyl, isoxazolinyl, isoxazolidinyl, isothiazolinyl, isothiazolidinyl, oxazolinyl, oxazolidinyl, thiazolinyl, thiazolidinyl, triazolinyl, triazolidinyl, oxadiazolinyl, oxadiazolidinyl, thiadiazolinyl, thiadiazolidinyl, tetrazolinyl, tetrazolidinyl, pyranyl, dihydro-2H-pyranyl, tetrahydropyranyl, thiopyranyl, 1,3-dioxanyl, 1,3-oxazinanyl, 1,2,5,6-tetrahydropyridinyl, 1,2,3,6-tetrahydropyridinyl, piperidinyl, piperazinyl, morpholinyl, thiomorpholinyl, 1,4-diazepanyl, 1,3-benzodioxolyl, 1,4-benzodioxanyl and the like. A heterocyclyl radical is optionally substituted on a carbon or nitrogen atom ring member with substituent species as described herein where allowed by available valences.

As used herein, the term “cyano” refers to a radical of the formula: —CN.

As used herein, the term “amino” refers to a radical of the formula: —NH₂.

As used herein, the term “C₁₋₆alkyl-amino” refers to a radical of the formula: —NH—C₁₋₆alkyl.

As used herein, the term “halo-C₁₋₆alkyl-amino” refers to a radical of the formula: —NH—C₁₋₆alkyl, wherein C₁₋₆alkyl is partially or completely substituted with one or more halogen atoms where allowed by available valences.

As used herein, the term “deutero-C₁₋₆alkyl-amino” refers to a radical of the formula: —NH—C₁₋₆alkyl, wherein C₁₋₆alkyl is partially or completely substituted with one or more deuterium atoms where allowed by available valences.

As used herein, the term “(C₁₋₆alkyl)₂-amino” refers to a radical of the formula: —N(C₁₋₆alkyl)₂.

As used herein, the term “phenyl-amino” refers to a radical of the formula: —NH-phenyl.

As used herein, the term “heterocyclyl-amino” refers to a radical of the formula: —NH-heterocyclyl.

As used herein, the term “heteroaryl-amino” refers to a radical of the formula: —NH-heteroaryl.

As used herein, the term “C₁₋₆alkyl-thio” refers to a radical of the formula: —S—C₁₋₆alkyl.

As used herein, the term “C₁₋₆alkyl-sulfonyl” refers to a radical of the formula: —SO₂—C₁₋₆alkyl.

As used herein, the term “halo” or “halogen” generally refers to a halogen atom radical, including fluoro, chloro, bromo and iodo.

As used herein, the term “halo-C₁₋₆alkoxy” refers to a radical of the formula: —O—C₁₋₆alkyl-halo, wherein C₁₋₆alkyl is partially or completely substituted with one or more halogen atoms where allowed by available valences.

As used herein, the term “halo-C₁₋₆alkyl” refers to a radical of the formula: —C₁₋₆alkyl-halo, wherein C₁₋₆alkyl is partially or completely substituted with one or more halogen atoms where allowed by available valences.

As used herein, the term “deutero-C₁₋₆alkyl” refers to a radical of the formula: —C₁₋₆alkyl-deutero, wherein C₁₋₆alkyl is partially or completely substituted with one or more deuterium atoms where allowed by available valences.

As used herein, the term “hydroxy” refers to a radical of the formula: —OH.

As used herein, the term “hydroxy-C₁₋₆alkyl” refers to a radical of the formula: —C₁₋₆alkyl-OH, wherein C₁₋₆alkyl is partially or completely substituted with one or more hydroxy radicals where allowed by available valences.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances, one or more substituents having a double bond (e.g., “oxo” or “═O”) as the point of attachment may be described, shown or listed herein within a substituent group, wherein the structure may only show a single bond as the point of attachment to the core structure of Formula (I). A person of ordinary skill in the art would understand that, while only a single bond is shown, a double bond is intended for those substituents.

As used herein, the term “and the like,” with reference to the definitions of chemical terms provided herein, means that variations in chemical structures that could be expected by one skilled in the art include, without limitation, isomers (including chain, branching or positional structural isomers), hydration of ring systems (including saturation or partial unsaturation of monocyclic, bicyclic or polycyclic ring structures) and all other variations where allowed by available valences which result in a stable compound.

For the purposes of this description, where one or more substituent variables for a compound of Formula (I) or a form thereof encompass functionalities incorporated into a compound of Formula (I), each functionality appearing at any location within the disclosed compound may be independently selected, and as appropriate, independently and/or optionally substituted.

As used herein, the terms “independently selected,” or “each selected” refer to functional variables in a substituent list that may occur more than once on the structure of Formula (I), the pattern of substitution at each occurrence is independent of the pattern at any other occurrence.

Further, the use of a generic substituent variable on any formula or structure for a compound described herein is understood to include the replacement of the generic substituent with species substituents that are included within the particular genus, e.g., aryl may be replaced with phenyl or naphthalenyl and the like, and that the resulting compound is to be included within the scope of the compounds described herein.

As used herein, the terms “each instance of” or “in each instance, when present,” when used preceding a phrase such as “ . . . C₃₋₁₀cycloalkyl, C₃₋₁₀cycloalkyl-C₁₋₄alkyl, aryl, aryl-C₁₋₄alkyl, heteroaryl, heteroaryl-C₁₋₄alkyl, heterocyclyl and heterocyclyl-C₁₋₄alkyl,” are intended to refer to the C₃₋₁₀cycloalkyl, aryl, heteroaryl and heterocyclyl ring systems when each are present either alone or as a substituent.

As used herein, the term “optionally substituted” means optional substitution with the specified substituent variables, groups, radicals or moieties.

### Compound Forms

As used herein, the term “form” means a compound of Formula (I) having a form selected from the group consisting of a free acid, free base, prodrug, salt, hydrate, solvate, clathrate, isotopologue, racemate, enantiomer, diastereomer, stereoisomer, polymorph and tautomer form thereof.

In certain aspects described herein, the form of the compound of Formula (I) is a free acid, free base or salt thereof.

In certain aspects described herein, the form of the compound of Formula (I) is a salt thereof.

In certain aspects described herein, the form of the compound of Formula (I) is an isotopologue thereof.

In certain aspects described herein, the form of the compound of Formula (I) is a stereoisomer, racemate, enantiomer or diastereomer thereof.

In certain aspects described herein, the form of the compound of Formula (I) is a tautomer thereof.

In certain aspects described herein, the form of the compound of Formula (I) is a pharmaceutically acceptable form.

In certain aspects described herein, the compound of Formula (I) or a form thereof is isolated for use.

As used herein, the term “isolated” means the physical state of a compound of Formula (I) or a form thereof after being isolated and/or purified from a synthetic process (e.g., from a reaction mixture) or natural source or combination thereof according to an isolation or purification process or processes described herein or which are well known to the skilled artisan (e.g., chromatography, recrystallization and the like) in sufficient purity to be characterized by standard analytical techniques described herein or well known to the skilled artisan.

As used herein, the term “protected” means that a functional group in a compound of Formula (I) or a form thereof is in a form modified to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York. Such functional groups include hydroxy, phenol, amino and carboxylic acid. Suitable protecting groups for hydroxy or phenol include trialkylsilyl or diarylalkylsilyl (e.g., t-butyldimethylsilyl, t-butyldiphenylsilyl or trimethylsilyl), tetrahydropyranyl, benzyl, substituted benzyl, methyl, methoxymethanol, and the like. Suitable protecting groups for amino, amidino and guanidino include t-butoxycarbonyl, benzyloxycarbonyl, and the like. Suitable protecting groups for carboxylic acid include alkyl, aryl or arylalkyl esters. In certain instances, the protecting group may also be a polymer resin, such as a Wang resin or a 2-chlorotrityl-chloride resin. Protecting groups may be added or removed in accordance with standard techniques, which are well-known to those skilled in the art and as described herein. It will also be appreciated by those skilled in the art, although such protected derivatives of compounds described herein may not possess pharmacological activity as such, they may be administered to a subject and thereafter metabolized in the body to form compounds described herein which are pharmacologically active. Such derivatives may therefore be described as “prodrugs”. All prodrugs of compounds described herein are included within the scope of the use described herein.

As used herein, the term “prodrug” means a form of an instant compound (e.g., a drug precursor) that is transformed in vivo to yield an active compound of Formula (I) or a form thereof. The transformation may occur by various mechanisms (e.g., by metabolic and/or non-metabolic chemical processes), such as, for example, by hydrolysis and/or metabolism in blood, liver and/or other organs and tissues. A discussion of the use of prodrugs is provided by T. Higuchi and W. Stella, “Pro-drugs as Novel Delivery Systems,” Vol. 14-of the A.C.S. Symposium Series, and in Bioreversible Carriers in Drug Design, ed. Edward B. Roche, American Pharmaceutical Association and Pergamon Press, 1987.

In one example, when a compound of Formula (I) or a form thereof contains a carboxylic acid functional group, a prodrug can comprise an ester formed by the replacement of the hydrogen atom of the acid group with a functional group such as alkyl and the like. In another example, when a compound of Formula (I) or a form thereof contains a hydroxyl functional group, a prodrug form can be prepared by replacing the hydrogen atom of the hydroxyl with another functional group such as alkyl, alkylcarbonyl or a phosphonate ester and the like. In another example, when a compound of Formula (I) or a form thereof contains an amine functional group, a prodrug form can be prepared by replacing one or more amine hydrogen atoms with a functional group such as alkyl or substituted carbonyl. Pharmaceutically acceptable prodrugs of compounds of Formula (I) or a form thereof include those compounds substituted with one or more of the following groups: carboxylic acid esters, sulfonate esters, amino acid esters, phosphonate esters and mono-, di- or triphosphate esters or alkyl substituents, where appropriate. As described herein, it is understood by a person of ordinary skill in the art that one or more of such substituents may be used to provide a compound of Formula (I) or a form thereof as a prodrug.

One or more compounds described herein may exist in unsolvated as well as solvated forms with pharmaceutically acceptable solvents such as water, ethanol, and the like, and the description herein is intended to embrace both solvated and unsolvated forms.

As used herein, the term “solvate” means a physical association of a compound described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. As used herein, “solvate” encompasses both solution-phase and isolatable solvates. Non-limiting examples of suitable solvates include ethanolates, methanolates, and the like.

As used herein, the term “hydrate” means a solvate wherein the solvent molecule is water.

The compounds of Formula (I) can form salts, which are intended to be included within the scope of this description. Reference to a compound of Formula (I) or a form thereof herein is understood to include reference to salt forms thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) or a form thereof contains both a basic moiety, such as, without limitation an amine moiety, and an acidic moiety, such as, but not limited to a carboxylic acid, zwitterions (“inner salts”) may be formed and are included within the term “salt(s)” as used herein.

The term “pharmaceutically acceptable salt(s)”, as used herein, means those salts of compounds described herein that are safe and effective (i.e., non-toxic, physiologically acceptable) for use in mammals and that possess biological activity, although other salts are also useful. Salts of the compounds of the Formula (I) may be formed, for example, by reacting a compound of Formula (I) or a form thereof with an amount of acid or base, such as an equivalent amount, in a medium such as one in which the salt precipitates or in an aqueous medium followed by lyophilization.

Pharmaceutically acceptable salts include one or more salts of acidic or basic groups present in compounds described herein. Particular aspects of acid addition salts include, and are not limited to, acetate, ascorbate, benzoate, benzenesulfonate, bisulfate, bitartrate, borate, bromide, butyrate, chloride, citrate, camphorate, camphorsulfonate, ethanesulfonate, formate, fumarate, gentisinate, gluconate, glucaronate, glutamate, iodide, isonicotinate, lactate, maleate, methanesulfonate, naphthalenesulfonate, nitrate, oxalate, pamoate, pantothenate, phosphate, propionate, saccharate, salicylate, succinate, sulfate, tartrate, thiocyanate, toluenesulfonate (also known as tosylate), trifluoroacetate salts and the like. Certain particular aspects of acid addition salts include chloride or dichloride.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33, 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Suitable basic salts include, but are not limited to, aluminum, ammonium, calcium, lithium, magnesium, potassium, sodium and zinc salts.

All such acid salts and base salts are intended to be included within the scope of pharmaceutically acceptable salts as described herein. In addition, all such acid and base salts are considered equivalent to the free forms of the corresponding compounds for purposes of this description.

Compounds of Formula (I) and forms thereof, may further exist in a tautomeric form. All such tautomeric forms are contemplated and intended to be included within the scope of the compounds of Formula (I) or a form thereof as described herein.

The compounds of Formula (I) or a form thereof may contain asymmetric or chiral centers, and, therefore, exist in different stereoisomeric forms. The present description is intended to include all stereoisomeric forms of the compounds of Formula (I) as well as mixtures thereof, including racemic mixtures.

The compounds described herein may include one or more chiral centers, and as such may exist as racemic mixtures (R S) or as substantially pure enantiomers and diastereomers. The compounds may also exist as substantially pure (R) or (S) enantiomers (when one chiral center is present). In one particular aspect, the compounds described herein are (S) isomers and may exist as enantiomerically pure compositions substantially comprising only the (S) isomer. In another particular aspect, the compounds described herein are (R) isomers and may exist as enantiomerically pure compositions substantially comprising only the (R) isomer. As one of skill in the art will recognize, when more than one chiral center is present, the compounds described herein may also exist as a (R,R), (R,S), (S,R) or (S,S) isomer, as defined by IUPAC Nomenclature Recommendations.

As used herein, the term “chiral” refers to a carbon atom bonded to four nonidentical substituents. Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. In describing an optically active compound, the prefixes D and L, or R and S, are used to denote the absolute configuration of the molecule about its chiral center(s). The substituents attached to the chiral center under consideration are ranked in accordance with the Sequence Rule of Cahn, Ingold and Prelog. (Cahn et al. Angew. Chem. Inter. Edit. 1966, 5, 385; errata 511).

As used herein, the term “substantially pure” refers to compounds consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of the single isomer.

In one aspect of the description, a compound of Formula (I) or a form thereof is a substantially pure (S) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

In one aspect of the description, a compound of Formula (I) or a form thereof is a substantially pure (R) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

As used herein, a “racemate” is any mixture of isometric forms that are not “enantiomerically pure”, including mixtures such as, without limitation, in a ratio of about 50/50, about 60/40, about 70/30, or about 80/20.

In addition, the present description embraces all geometric and positional isomers. For example, if a compound of Formula (I) or a form thereof incorporates a double bond or a fused ring, both the cis- and trans-forms, as well as mixtures, are embraced within the scope of the description. Diastereomeric mixtures can be separated into their individual diastereomers on the basis of their physical chemical differences by methods well known to those skilled in the art, such as, for example, by chromatography and/or fractional crystallization. Enantiomers can be separated by use of chiral HPLC column or other chromatographic methods known to those skilled in the art. Enantiomers can also be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this description.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this description, as are positional isomers (such as, for example, 4-pyridyl and 3-pyridyl). Individual stereoisomers of the compounds described herein may, for example, be substantially free of other isomers, or may be present in a racemic mixture, as described supra.

### Compound Uses

Provided herein are methods of treating a disease in a subject in need thereof. As used herein, the terms “subject” or “patient” refer to any animal, including mammals. For example, mice, rats, other rodents, rabbits, dogs, cats, swine, cattle, sheep, horses, primates, and humans. In some aspects, the subject is a human.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician. In some aspects, the dosage of the compound, or a pharmaceutically acceptable salt thereof, administered to a subject or individual is about 1 mg to about 2 g, about 1 mg to about 1000 mg, about 1 mg to about 500 mg, about 1 mg to about 100 mg, about 1 mg to 50 mg, or about 50 mg to about 500 mg.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

The present application provides a method of treating a neurodegenerative disease characterized by the accumulation and aggregation of aberrant forms MAPT in a subject in need thereof, comprising administering to the subject a therapeutically effective amount of a compound provided herein (i.e., a compound of Formula (I)).

Also provided herein is a method of treating the formation of neurofibrillary tangles and paired helical filaments in neurons and glia in a subject in need thereof, comprising administering to the subject a therapeutically effective amount of a compound provided herein (i.e., a compound of Formula (I)).

Also provided herein are methods of lowering MAPT4R protein in a subject, comprising administering to the subject a therapeutically effective amount of a compound provided herein (i.e., a compound of Formula (I)).

In some aspects of the methods provided herein, the compound is selected from the group of compounds of Formula (I) or a pharmaceutically acceptable salt thereof.

Also provided herein are methods of inducing exon 10 skipping in MAPT pre-mRNA in a subject, comprising administering to a subject an effective amount of a compound of Formula (I) or form thereof.

Also provided herein are methods of inducing exon 10 skipping in MAPT pre-mRNA in a cell, comprising contacting a cell (e.g. ex vivo or in vivo) with a compound of Formula (I) or form thereof.

Also provided herein are methods of inducing exon 10 skipping in MAPT pre-mRNA in a gene comprising contacting the gene (e.g., in a cell or subject expressing the gene) with a compound a compound of Formula (I) or a form thereof.

Also provided therein are methods of producing MAPT ΔE4 mRNA in a subject in need thereof, the method comprising administering an effective amount of a compound Formula (I) or a form thereof to the subject.

Also provided therein are methods of producing MAPT ΔE4 mRNA in a cell, the method comprising contacting the cell (e.g. ex vivo or in vivo) with a compound Formula (I) or a form thereof to the subject.

Also provided herein are methods of producing MAPT ΔE4 mRNA in a gene comprising contacting the gene (e.g., in a cell or subject expressing the gene) with a compound a compound of Formula (I) or a form thereof.

Also provided herein are methods for decreasing MAPT 4R mRNA in a subject in need thereof, the method comprising administering an effective amount of a compound of Formula (I) or a form thereof to the subject. For example, such methods include decreasing MAPT 4R mRNA concentration in serum samples from the subject.

In some aspects, MAPT 4R mRNA can be measured in the serum, for example, in blood samples obtained from the subject prior to administration of a compound of Formula (I) or form thereof and in blood samples obtained from the subject following administration of a compound as provided herein. In some aspects, the blood samples obtained from the subject following administration are obtained after one day, two days, three days, four days, five days, six days, seven days, eight days, nine days, ten days, fourteen days, twenty-one days, twenty-eight days, and/or thirty days of administration of the compound as provided herein. See, for example, F. B. Axelrod et al., Pediatr Res (2011) 70(5): 480-483; and R. S. Shetty et al., Human Molecular Genetics (2011) 20(21): 4093-4101, both of which are incorporated by reference in their entirety.

Further provided herein is a method for decreasing MAPT 4R mRNA in a cell, the method comprising contacting the cell (e.g. ex vivo or in vivo) with a therapeutically effective amount of a compound of Formula (I) or a form salt thereof. The amount of MAPT 4R mRNA in the treated cell is decreased relative to a cell in a subject in the absence of a compound provided herein. The method for decreasing the amount of MAPT 4R mRNA in a cell may be performed by contacting the cell with a compound of Formula (I) or a form thereof in vitro, thereby decreasing the amount of MAPT 4R mRNA of a cell in vitro. Uses of such an in vitro method of decreasing the amount of MAPT 4R mRNA include, but are not limited to, use in a screening assay (for example, wherein a compound of Formula (I) or a form thereof is used as a positive control or standard compared to a compound or compounds of unknown activity or potency in decreasing the amount MAPT 4R mRNA).

In some aspects, the amount of MAPT 4R mRNA is decreased in a central nervous system cell. In some aspects thereof, the amount of MAPT 4R mRNA is decreased in the plasma.

The method of decreasing mutant MAPT 4R mRNA in a central nervous system cell may be performed, for example, by contacting a cell with a compound of Formula (I) or a form thereof in vivo, thereby decreasing the amount of MAPT 4R mRNA in a subject in vivo. The contacting is achieved by causing a compound of Formula (I) or a form thereof to be present in a subject in an amount effective to achieve a decrease in the amount of MAPT 4R mRNA. This may be achieved, for example, by administering an effective amount of a compound of Formula (I) or a form thereof to a subject. Uses of such an in vivo method of decreasing the amount of MAPT 4R mRNA include, but are not limited to, use in methods of treating a disease or condition, wherein a decrease in the amount of MAPT 4R mRNA is beneficial.

In some aspects thereof, the amount of MAPT 4R mRNA is decreased in a central nervous system cell in a subject suffering from a neurodegenerative disease characterized by the accumulation and aggregation of aberrant forms of MAPT. The method is preferably performed by administering an effective amount of a compound of Formula (I) or a form thereof to a subject who is suffering from a neurodegenerative disease characterized by the accumulation and aggregation of aberrant forms of MAPT.

Also provided herein are methods for decreasing MAPT4R protein expression in a subject in need thereof, the method comprising administering an effective amount of a compound of Formula (I) or a pharmaceutically acceptable salt thereof to the subject. For example, such methods include decreasing MAPT4R protein expression in serum samples from the subject. Further provided herein are methods for decreasing the mean percentage of MAPT4R protein expression in a subject in need thereof, the method comprising administering an effective amount of a compound of Formula (I) or a form thereof to the subject.

Also provided herein are methods for decreasing MAPT4R protein level in a subject in need thereof, the method comprising administering an effective amount of a compound of Formula (I) or a form thereof to the subject. Further provided herein are methods for decreasing the mean percentage of MAPT4R protein level in a subject in need thereof, the method comprising administering an effective amount of a compound of Formula (I) or a form thereof, to the subject.

Also provided herein are methods for decreasing MAPT4R protein level in a cell (e.g., ex vivo or in vivo), the method comprising contacting the cell with a therapeutically effective amount of a compound of Formula (I) or a form thereof.

In some aspects, the method is an in vitro method. In some aspects, the method is an in vivo method. In some aspects, the amount of MAPT4R protein level is decreased in a cell. In some aspects, the cell is a central nervous system cell. In some aspects, one or more of the compounds of Formula (I) or form thereof may be administered to a subject in need thereof in combination with at least one additional pharmaceutical agent.

Additional examples of suitable additional pharmaceutical agents for use in combination with the compounds of the present application for treatment of the diseases provided herein include, but are not limited to, antioxidants, anti-inflammatory agents, steroids, immunosuppressants, or other agents such as therapeutic antibodies. In some aspects, the compounds of Formula (I) or a form thereof may be administered to a subject in need thereof in combination with at least one additional pharmaceutical agent for the treatment of a neurodegenerative disease characterized by the accumulation and aggregation of aberrant forms of MAPT.

When employed as a therapeutic agent, the compounds provided herein can be administered in the form of a pharmaceutical composition; thus, the methods described herein can include administering a pharmaceutical composition. These compositions can be prepared as described herein or elsewhere, and can be administered by a variety of routes, depending upon whether local or systemic treatment is desired and upon the area to be treated. Administration may be pulmonary (e.g., by inhalation or insufflation of powders or aerosols, including by nebulizer; intratracheal or intranasal), oral, or parenteral. Parenteral administration may include, but is not limited to intravenous, intraarterial, subcutaneous, intraperitoneal, intramuscular injection or infusion; or intracranial, (e.g., intrathecal, intraocular, or intraventricular) administration. Parenteral administration can be in the form of a single bolus dose, or may be, for example, by a continuous perfusion pump. Conventional pharmaceutical carriers, aqueous, powder or oily bases, thickeners and the like may be necessary or desirable. In some aspects, the compounds provided herein are suitable for oral and parenteral administration. In some aspects, the compounds provided herein are suitable for oral administration. In some aspects, the compounds provided herein are suitable for parenteral administration. In some aspects, the compounds provided herein are suitable for intravenous administration. In some aspects, the compounds provided herein are suitable for transdermal administration (e.g., administration using a patch or microneedle). Pharmaceutical compositions for topical administration may include transdermal patches (e.g., normal or electrostimulated), ointments, lotions, creams, gels, drops, suppositories, sprays, liquids and powders. Conventional pharmaceutical carriers, aqueous, powder or oily bases, thickeners and the like may be necessary or desirable.

Also provided are pharmaceutical compositions which contain, as the active ingredient, a compound of Formula (I) or a form thereof in combination with one or more pharmaceutically acceptable carriers (excipients). In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil; wetting agents; emulsifying and suspending agents; preserving agents such as methyl-and propylhydroxy-benzoates; sweetening agents; flavoring agents, or combinations thereof.

The active compound can be effective over a wide dosage range and is generally administered in a pharmaceutically effective amount. It will be understood that the amount of compound to be administered and the schedule of administration will usually be determined by a physician, according to the relevant circumstances, including the condition to be treated, the chosen route of administration, the actual compound administered, the age, weight, and response of the individual subject, the severity of the subject's symptoms, and the like.

In another aspect, the concentration-biological effect relationship observed with regard to a compound of Formula (I) or a form thereof indicate a target plasma concentration ranging from approximately 0.001 μg·hr/mL to approximately 50 μg·hr/mL, from approximately 0.01 μg·hr/mL to approximately 20 μg·hr/mL, from approximately 0.05 μg·hr/mL to approximately 10 μg·hr/mL, or from approximately 0.1 μg·hr/mL to approximately 5 μg·hr/mL. To achieve such plasma concentrations, the compounds described herein may be administered at doses that vary, such as, for example, without limitation, from 1.0 ng to 10,000 mg.

In one aspect, the dose administered to achieve an effective target plasma concentration may be administered based upon subject or patient specific factors, wherein the doses administered on a weight basis may be in the range of from about 0.001 mg/kg/day to about 3500 mg/kg/day, or about 0.001 mg/kg/day to about 3000 mg/kg/day, or about 0.001 mg/kg/day to about 2500 mg/kg/day, or about 0.001 mg/kg/day to about 2000 mg/kg/day, or about 0.001 mg/kg/day to about 1500 mg/kg/day, or about 0.001 mg/kg/day to about 1000 mg/kg/day, or about 0.001 mg/kg/day to about 500 mg/kg/day, or about 0.001 mg/kg/day to about 250 mg/kg/day, or about 0.001 mg/kg/day to about 200 mg/kg/day, or about 0.001 mg/kg/day to about 150 mg/kg/day, or about 0.001 mg/kg/day to about 100 mg/kg/day, or about 0.001 mg/kg/day to about 75 mg/kg/day, or about 0.001 mg/kg/day to about 50 mg/kg/day, or about 0.001 mg/kg/day to about 25 mg/kg/day, or about 0.001 mg/kg/day to about 10 mg/kg/day, or about 0.001 mg/kg/day to about 5 mg/kg/day, or about 0.001 mg/kg/day to about 1 mg/kg/day, or about 0.001 mg/kg/day to about 0.5 mg/kg/day, or about 0.001 mg/kg/day to about 0.1 mg/kg/day, or from about 0.01 mg/kg/day to about 3500 mg/kg/day, or about 0.01 mg/kg/day to about 3000 mg/kg/day, or about 0.01 mg/kg/day to about 2500 mg/kg/day, or about 0.01 mg/kg/day to about 2000 mg/kg/day, or about 0.01 mg/kg/day to about 1500 mg/kg/day, or about 0.01 mg/kg/day to about 1000 mg/kg/day, or about 0.01 mg/kg/day to about 500 mg/kg/day, or about 0.01 mg/kg/day to about 250 mg/kg/day, or about 0.01 mg/kg/day to about 200 mg/kg/day, or about 0.01 mg/kg/day to about 150 mg/kg/day, or about 0.01 mg/kg/day to about 100 mg/kg/day, or about 0.01 mg/kg/day to about 75 mg/kg/day, or about 0.01 mg/kg/day to about 50 mg/kg/day, or about 0.01 mg/kg/day to about 25 mg/kg/day, or about 0.01 mg/kg/day to about 10 mg/kg/day, or about 0.01 mg/kg/day to about 5 mg/kg/day, or about 0.01 mg/kg/day to about 1 mg/kg/day, or about 0.01 mg/kg/day to about 0.5 mg/kg/day, or about 0.01 mg/kg/day to about 0.1 mg/kg/day, or from about 0.1 mg/kg/day to about 3500 mg/kg/day, or about 0.1 mg/kg/day to about 3000 mg/kg/day, or about 0.1 mg/kg/day to about 2500 mg/kg/day, or about 0.1 mg/kg/day to about 2000 mg/kg/day, or about 0.1 mg/kg/day to about 1500 mg/kg/day, or about 0.1 mg/kg/day to about 1000 mg/kg/day, or about 0.1 mg/kg/day to about 500 mg/kg/day, or about 0.1 mg/kg/day to about 250 mg/kg/day, or about 0.1 mg/kg/day to about 200 mg/kg/day, or about 0.1 mg/kg/day to about 150 mg/kg/day, or about 0.1 mg/kg/day to about 100 mg/kg/day, or about 0.1 mg/kg/day to about 75 mg/kg/day, or about 0.1 mg/kg/day to about 50 mg/kg/day, or about 0.1 mg/kg/day to about 25 mg/kg/day, or about 0.1 mg/kg/day to about 10 mg/kg/day, or about 0.1 mg/kg/day to about 5 mg/kg/day, or about 0.1 mg/kg/day to about 1 mg/kg/day, or about 0.1 mg/kg/day to about 0.5 mg/kg/day.

Effective amounts for a given subject may be determined by routine experimentation that is within the skill and judgment of a clinician or a practitioner skilled in the art in light of factors related to the subject. Dosage and administration may be adjusted to provide sufficient levels of the active agent(s) or to maintain the desired effect. Factors which may be taken into account include genetic screening, severity of the disease state, status of disease progression, general health of the subject, ethnicity, age, weight, gender, diet, time of day and frequency of administration, drug combination(s), reaction sensitivities, experience with other therapies, and tolerance/response to therapy.

The dose administered to achieve an effective target plasma concentration may be orally administered once (once in approximately a 24 hour period; i.e., “q.d.”), twice (once in approximately a 12 hour period; i.e., “b.i.d.” or “q.12 h”), thrice (once in approximately an 8 hour period; i.e., “t.i.d.” or “q.8 h”), or four times (once in approximately a 6 hour period; i.e., “q.d.s.”, “q.i.d.” or “q.6 h”) daily.

In certain aspects, the dose administered to achieve an effective target plasma concentration may also be administered in a single, divided, or continuous dose for a patient or subject having a weight in a range of between about 40 to about 200 kg (which dose may be adjusted for patients or subjects above or below this range, particularly children under 40 kg). The typical adult subject is expected to have a median weight in a range of about 70 kg. Long-acting pharmaceutical compositions may be administered every 2, 3 or 4 days, once every week, or once every two weeks depending on half-life and clearance rate of the particular formulation.

The compounds and compositions described herein may be administered to the subject via any drug delivery route known in the art. Nonlimiting examples include oral, ocular, rectal, buccal, topical, nasal, sublingual, transdermal, subcutaneous, intramuscular, intraveneous (bolus and infusion), intracerebral, and pulmonary routes of administration.

In another aspect, the dose administered may be adjusted based upon a dosage form described herein formulated for delivery at about 0.02, 0.025, 0.03, 0.05, 0.06, 0.075, 0.08, 0.09, 0.10, 0.20, 0.25, 0.30, 0.50, 0.60, 0.75, 0.80, 0.90, 1.0, 1.10, 1.20, 1.25, 1.50, 1.75, 2.0, 3.0, 5.0, 10, 20, 30, 40, 50, 100, 150, 200, 250, 300, 400, 500, 1000, 1500, 2000, 2500, 3000 or 4000 mg/day.

For any compound, the effective amount can be estimated initially either in cell culture assays or in relevant animal models, such as a mouse, guinea pig, chimpanzee, marmoset or tamarin animal model. Relevant animal models may also be used to determine the appropriate concentration range and route of administration. Such information can then be used to determine useful doses and routes for administration in humans. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is therapeutic index, and can be expressed as the ratio, LD₅₀/ED₅₀. In certain aspects, the effective amount is such that a large therapeutic index is achieved. In further particular aspects, the dosage is within a range of circulating concentrations that include an ED₅₀ with little or no toxicity. The dosage may vary within this range depending upon the dosage form employed, sensitivity of the patient, and the route of administration.

Another aspect included within the scope of the present description are the use of in vivo metabolic products of the compounds described herein. Such products may result, for example, from the oxidation, reduction, hydrolysis, amidation, esterification and the like of the administered compound, primarily due to enzymatic processes. Accordingly, the description includes the use of compounds produced by a process comprising contacting a compound described herein with a mammalian tissue or a mammal for a period of time sufficient to yield a metabolic product thereof.

Such products typically are identified by preparing a radio-labeled (e.g., ¹⁴C or ³H) compound of Formula (I), administering the radio-labeled compound in a detectable dose (e.g., greater than about 0.5 mg/kg) to a mammal such as a rat, mouse, guinea pig, dog, monkey or human, allowing sufficient time for metabolism to occur (typically about 30 seconds to about 30 hours), and identifying the metabolic conversion products from urine, bile, blood or other biological samples. The conversion products are easily isolated since they are “radiolabeled” by virtue of being isotopically-enriched (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds described herein even if they possess no biological activity of their own.

### Preparation of Compounds

Compounds of Formula (I) can be prepared using reagents and methods known in the art, including the methods provided in International Application No. PCT/US2020/063612, the entire contents of which are incorporated herein by reference.

### Biological Examples

The following in vitro biological examples demonstrate the usefulness of the compounds of the present description for treating neurodegenerative diseases characterized by the accumulation of aberrant forms of MAPT.

To describe in more detail and assist in understanding the present description, the following non-limiting biological examples are offered to more fully illustrate the scope of the description and are not to be construed as specifically limiting the scope thereof. Such variations of the present description that may be now known or later developed, which would be within the purview of one skilled in the art to ascertain, are considered to fall within the scope of the present description and as hereinafter claimed.

### Example 1

RT-qPCR Assay to Quantify MAPT 3R mRNA in Cells

Test compounds were serially diluted 3.16-fold in 100% DMSO to generate a 7-point concentration curve. Aliquots of 0.5 μL of diluted compounds were transferred to a 96-well flat bottom plate by a liquid handler. An aliquot of 0.5 μL DMSO was also transferred to separate wells and used as controls. Duplicate samples were set up for each compound concentration and for the DMSO control.

Cells were thawed and incubated in cell culture media (DMEM, 1 FBS, and No antibiotic cocktail) for 72 h. Cells were trypsinized, counted, and re-suspended to a concentration of 200,000 cells/mL in cell culture media. A 100 μL aliquot of the cell suspensions were plated at 20,000 cells per well in the compound containing 96 well microtiter plate and incubated for in a cell culture incubator (37° C., 5% C₂, 100% relative humidity).

After 24 h, media was aspirated from the cells and 20 μL of the RCL2 lysis buffer (10 mM Tris-HCL pH 7.4, 150 mM NaCl, 0.3300 IGEPAL® CA-630) was added to each well and incubated at RT for 1 mi. Chilled nuclease free water (140 μL per well) was added and the plates were immediately transferred on ice. After 1 min on ice, plates were frozen at −80° C. overnight.

### Preparation of RT-PCR Reaction Mixture:

| | Volume | Supplier and |
|---|---|---|
| Reagent | (μL) | Catalogue No. |
| RT-PCR buffer (2×) | 5.0 | Thermo Fisher, |
| | | 4387391 |
| RT-PCR enzyme | 0.4 | Thermo Fisher, |
| mixture (25×) | | 4387391 |
| in house MAPT 3R | 0.5 | |
| Primer/Probe (20×) | | |
| In house GAPDH | 0.5 | |
| assay (20×) | | |
| H₂O | 1.94 | |

Abbreviations:
GAPDH, glyceraldehyde 3-phosphate dehydrogenase
Target: Microtubule associated protein Tau
In house GAPDH assay:
Forward primer-5′ caacggatttggtcgtattgg 3′
Reverse primer-5′ tgatggcaacaatatccactttacc 3′
Probe (VIC-TAMRA)-5′ cgcctggtcaccagggctgct 3′
In house MAPT minigene 3R assay:
Forward primer-5′ AGGCGGGAAGGTGCAAATA 3′
Reverse primer-5′ CTGGTTTATGATGGATGTTGCCT 3′
Probe (FAM-MGB)-5′ TCTACAAACCAGTTGACCTGAGCAAGGTGACC 3′

An aliquot of 4 μL/well of the cell lysates was transferred using the liquid handler to the Armadillo 384-Well PCR plate containing 6 μL/well of the RT-qPCR reaction mixture that was prepared as detailed above. The plates were then sealed with MicroAmp™ Optical Adhesive Film followed by spinning down for 1 min and placed in the CFX384 thermocycler (BioRad).

The RT-qPCR was carried out at the following temperatures for the indicated time:
Step 1: 48° C. (30 min)
Step 2: 95° C. (10 min)
Step 3: 95° C. (15 sec)
Step 4: 60° C. (1 min);
   then, repeated Steps 3 and 4 for a total of 40 cycles.

The percent exon 4 skipping was calculated for each dose of compound treatment using Equations 1 and 2.

$$Realtive gene expression = \frac{{2}^{- Ct ( target )}}{{1.9}^{- Ct ( GAPDH )}} \qquad Equation 1$$

$$Equation 2$$

$$Percent exon 4 skipping ( \% ) = 1 - [ \frac{Realtive gene expression , Compound}{Realtive gene expression , DMSO} ] \times 100$$

Data were fit to a dose response curve and the EC₂ₓ was interpolated using XLfit® statistical and curve fitting package. The resulting EC₂ₓ values (μM) for the representative compounds tested are shown in Table 1.

An EC₂ₓ value≥3000 nM is indicated by “inactive.” An EC₂ₓ value between >1500 nM and ≤3000 nM is indicated by one star (*). An EC₂ₓ value between >1000 nM and ≤1500 nM is indicated by two stars (**). An EC₂ₓ value between >500 nM and ≤1000 nM is indicated by three stars (***). An EC₂ₓ value between >50 nM and ≤500 nM is indicated by four stars (****). An EC₂ₓ value≤50 nM is indicated by five stars (*****).

TABLE 1

| Cpd | EC_{2X} |
|---|---|
| 5 | **** |
| 12 | ***** |
| 15 | **** |
| 23 | **** |
| 24 | **** |
| 28 | ***** |
| 29 | ***** |
| 31 | * |
| 33 | **** |
| 38 | *** |
| 39 | **** |
| 51 | ***** |
| 56 | ***** |
| 59 | *** |
| 60 | * |
| 62 | **** |
| 64 | inactive |
| 72 | * |
| 74 | ***** |
| 75 | *** |
| 77 | **** |
| 78 | * |
| 82 | ***** |
| 86 | **** |
| 90 | **** |
| 92 | **** |
| 93 | **** |
| 97 | ***** |

Without regard to whether a document cited herein was specifically and individually indicated as being incorporated by reference, all documents referred to herein are incorporated by reference into the present application for any and all purposes to the same extent as if each individual reference was fully set forth herein.

Having now fully described the subject matter of the claims, it will be understood by those having ordinary skill in the art that the same can be performed within a wide range of equivalents without affecting the scope of the subject matter or particular aspects described herein. It is intended that the appended claims be interpreted to include all such equivalents.

1. A method for treating a neurodegenerative disease characterized by the accumulation of aberrant forms of the microtubule associated protein Tau (MAPT) in a subject in need thereof, comprising administering to said subject an effective amount of a compound of Formula (I): 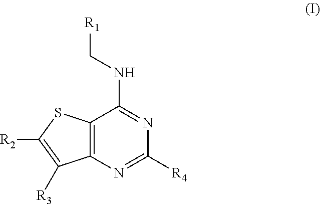
or a form thereof, wherein
R₁ is selected from the group consisting of phenyl and heteroaryl,
wherein heteroaryl is a 5-8 membered monocyclic or bicyclic aromatic carbon atom ring structure radical containing 1-3 heteroatoms selected from N, O, and S, and
wherein phenyl or heteroaryl is optionally substituted with one, two, three, or four, independently selected R₁ₐ substituents;
R₁ₐ is independently selected from the group consisting of cyano, halo, hydroxy, C₁₋₆alkyl, halo-C₁₋₆alkyl, deutero-C₁₋₆alkyl, and C₁₋₆alkoxy;
R₂ is selected from the group consisting of hydrogen, C₁₋₆alkyl, C₂₋₆alkenyl, C₂₋₆alkynyl, C₃₋₁₀cycloalkyl, phenyl, heterocyclyl, and heteroaryl,
wherein heterocyclyl is a 3-7 membered monocyclic carbon atom ring structure radical containing 1-3 heteroatoms selected from N, O, and S,
wherein heteroaryl is a 5-8 membered monocyclic or bicyclic aromatic carbon atom ring structure radical containing 1-3 heteroatoms selected from N, O, and S,
wherein C₁₋₆alkyl, C₂₋₆alkenyl, and C₂₋₆alkynyl optionally contain a chiral carbon having an (R) or (S) configuration, and
wherein C₁₋₆alkyl, C₂₋₆alkenyl, C₂₋₆alkynyl, C₃₋₁₀cycloalkyl, phenyl, heterocyclyl, or heteroaryl are optionally substituted with one, two, three, or four independently selected R₂ₐ substituents;
R₂ₐ is independently selected from the group consisting of cyano, halo, hydroxy, oxo, C₁₋₆alkyl, halo-C₁₋₆alkyl, deutero-C₁₋₆alkyl, C₂₋₆alkenyl, C₂₋₆alkynyl, C₁₋₆alkoxy, halo-C₁₋₆alkoxy, carboxyl, amino, C₁₋₆alkyl-amino, halo-C₁₋₆alkyl-amino, deutero-C₁₋₆alkyl-amino, (C₁₋₆alkyl)₂-amino, C₃₋₁₀cycloalkyl-amino, phenyl-amino, heterocyclyl-amino, heteroaryl-amino, C₁₋₆alkyl-thio, C₁₋₆alkyl-sulfonyl, C₃₋₁₀cycloalkyl, phenyl, heterocyclyl, and heteroaryl,
wherein heterocyclyl is a 3-7 membered monocyclic carbon atom ring structure radical containing 1-3 heteroatoms selected from N, O, and S,
wherein heteroaryl is a 5-8 membered monocyclic or bicyclic aromatic carbon atom ring structure radical containing 1-3 heteroatoms selected from N, O, and S, and
wherein each instance of C₃₋₁₀cycloalkyl, phenyl, heterocyclyl or heteroaryl is optionally substituted with one, two, three or four independently selected R_{2a′} substituents;
R_{2a′} is independently selected from the group consisting of cyano, halo, hydroxy, oxo, C₁₋₆alkyl, halo-C₁₋₆alkyl, deutero-C₁₋₆alkyl and C₁₋₆alkoxy;
R₃ is selected from the group consisting of hydrogen, cyano, halo, hydroxy, C₁₋₆alkyl, halo-C₁₋₆alkyl, C₁₋₆alkoxy, amino, C₁₋₆alkyl-amino, (C₁₋₆alkyl)₂-amino, C₃₋₁₀cycloalkyl, phenyl, heterocyclyl, and heteroaryl,
wherein heterocyclyl is a 3-7 membered monocyclic carbon atom ring structure radical containing 1-3 heteroatoms selected from N, O, and S, and
wherein heteroaryl is a 5-8 membered monocyclic or bicyclic aromatic carbon atom ring structure radical containing 1-3 heteroatoms selected from N, O, and S, and
wherein C₁₋₆alkyl, C₃₋₁₀cycloalkyl, phenyl, heterocyclyl, or heteroaryl is optionally substituted with one, two, three, or four independently selected R₃ₐ substituents;
R₃ₐ is independently selected from the group consisting of cyano, halo, hydroxy, C₁₋₆alkyl, halo-C₁₋₆alkyl, and C₁₋₆alkoxy;
R₄ is selected from the group consisting of hydrogen, cyano, halo, hydroxy, C₁₋₆alkyl, halo-C₁₋₆alkyl, C₁₋₆alkoxy, carbamoyl, C₃₋₁₀cycloalkyl, phenyl, and heterocyclyl, and
wherein heterocyclyl is a 3-7 membered monocyclic carbon atom ring structure radical containing 1-3 heteroatoms selected from N, O, and S; and
wherein the form of the compound is selected from the group consisting of a salt, hydrate, solvate, and tautomer form thereof.

2. The compound of claim 1, wherein R₁ is phenyl optionally substituted with one, two, three, or four independently selected R₁ₐ substituents.

3. The compound of claim 1, wherein R₁ is heteroaryl selected from the group consisting of furanyl, thiophenyl, 1H-pyrazolyl, 1H-imidazolyl, isoxazolyl, 1,2-thiazolyl, 1,3-thiazolyl, 1,2-oxazolyl, 1,3-oxazolyl, tetrazolyl, 1,2,3-triazolyl, 1,2,4-oxadiazolyl, 1,3,4-oxadiazolyl, 1,2,3-thiadiazolyl, pyridinyl, pyrimidinyl, pyrazinyl, pyridazinyl, benzofuranyl, and quinolinyl, wherein heteroaryl is optionally substituted with one, two, three, or four independently selected R₁ substituents.

4. The method of claim 1, wherein R₁ is heteroaryl selected from the group consisting of furanyl, thiophenyl, 1H-pyrazolyl, 1H-imidazolyl, 1,2-thiazolyl, 1,3-thiazolyl, 1,2-oxazolyl, 1,3-oxazolyl, pyridinyl, pyrimidinyl, and pyrazinyl.

5. The method of claim 1, wherein R₁ₐ is selected from the group consisting of halo and C₁₋₆alkyl.

6. The method of claim 1, wherein R₂ is selected from the group consisting of hydrogen, C₁₋₆alkyl, and heterocyclyl.

7. The method of claim 1, wherein R₂ is C₁₋₆alkyl selected from the group consisting of methyl, ethyl, propyl, butyl, and pentyl.

8. The method of claim 1, wherein R₂ is C₁₋₆alkyl, and wherein C₁₋₆alkyl contains a chiral carbon having the (R) configuration.

9. The method of claim 1, wherein R₂ is C₁₋₆alkyl, and wherein C₁₋₆alkyl contains a chiral carbon having the (S) configuration.

10. A method for treating a neurodegenerative disease characterized by the accumulation of aberrant forms of the microtubule associated protein Tau (MAPT) in a subject in need thereof, comprising administering to said subject an effective amount of a compound or form thereof selected form the group consisting of:
2-chloro-N-[(pyridin-4-yl)methyl]thieno[3,2-d]pyrimidin-4-amine;
2-chloro-N-[(furan-2-yl)methyl]thieno[3,2-d]pyrimidin-4-amine;
6-[(2S)-2-aminopropyl]-2-chloro-N-[(furan-2-yl)methyl]thieno[3,2-d]pyrimidin-4-amine;
6-[(2S)-2-aminobutyl]-2-chloro-N-[(furan-2-yl)methyl]thieno[3,2-d]pyrimidin-4-amine;
6-[(2R)-2-amino-3-methylbutyl]-2-choro-N-[(furan-2-yl)methyl]thieno[3,2-d]pyrimidin-4-amine;
6-[(2R,3S)-2-amino-3-methylpentyl]-2-chloro-N-[(furan-2-yl)methyl]thieno[3,2-d]pyrimidin-4-amine;
6-[(2R)-2-amino-3,3-dimethylbutyl]-2-chloro-N-[(furan-2-yl)methyl]thieno[3,2-d]pyrimidin-4-amine;
6-[(2S)-2-aminopropyl]-N-[(furan-2-yl)methyl]thieno[3,2-d]pyrimidin-4-amine;
6-[(2S)-2-aminopropyl]-2-chloro-N-[(pyridin-4-yl)methyl]thieno[3,2-d]pyrimidin-4-amine;
6-[(2S)-2-aminobutyl]-2-chloro-N-[(thiophen-2-yl)methyl]thieno[3,2-d]pyrimidin-4-amine;
6-[(2S)-2-aminopropyl]-N-[(pyridin-4-yl)methyl]thieno[3,2-d]pyrimidin-4-amine;
6-[(2S)-2-aminopropyl]-2-chloro-N-[(furan-2-yl)methyl]-7-methylthieno[3,2-d]pyrimidin-4-amine;
6-[(2S)-2-aminobutyl]-2-chloro-N-[(furan-2-yl)methyl]-7-methylthieno[3,2-d]pyrimidin-4-amine;
6-[(2S)-2-aminopropyl]-2-chloro-N-[(thiophen-2-yl)methyl]thieno[3,2-d]pyrimidin-4-amine;
6-[(2)-2-aminopropyl]-N-[(furan-2-yl)methyl]-2,7-dimethylthieno[3,2-d]pyrimidin-4-amine;
6-[(2S)-2-aminopropyl]-2-ethyl-N-[(furan-2-yl)methyl]-7-methylthieno[3,2-d]pyrimidin-4-amine;
6-[(2S)-2-aminopropyl]-2-cyclopropyl-N-[(furan-2-yl)methyl]-7-methylthieno[3,2-d]pyrimidin-4-amine;
2-chloro-N-[(furan-2-yl)methyl]-7-methylthieno[3,2-d]pyrimidin-4-amine;
(2R)-2-amino-3-(2-chloro-4-{[(furan-2-yl)methyl]amino}-7-methylthieno[3,2-d]pyrimidin-6-yl)propan-1-ol;
6-[(2S)-2-aminopropyl]-4-{[(furan-2-yl)methyl]amino}-7-methylthieno[3,2-d]pyrimidine-2-carboxamide;
6-[(2S)-2-aminopropyl]-4-{[(furan-2-yl)methyl]amino}-7-methylthieno[3,2-d]pyrimidine-2-carbonitrile;
(2R)-2-amino-3-(2-chloro-4-{[(furan-2-yl)methyl]amino}thieno[3,2-d]pyrimidin-6-yl)propan-1-ol;
2-chloro-7-methyl-N-[(pyridin-4-yl)methyl]thieno[3,2-d]pyrimidin-4-amine;
6-[(2S)-2-aminopropyl]-2-chloro-7-methyl-N-[(5-methylfuran-2-yl)methyl]thieno[3,2-d]pyrimidin-4-amine;
N-[(furan-2-yl)methyl]-7-methyl-2-(trifluoromethyl)thieno[3,2-d]pyrimidin-4-amine;
6-[(2S)-2-aminopropyl]-N-[(furan-2-yl)methyl]-7-methyl-2-(trifluoromethyl)thieno[3,2-d]pyrimidin-4-amine;
6-[(2S)-2-aminopropyl]-2-chloro-7-methyl-N-[(4-methyl-1,3-thiazol-2-yl)methyl]thieno[3,2-d]pyrimidin-4-amine;
6-[(2S)-2-aminopropyl]-2-chloro-7-methyl-N-[(thiophen-2-yl)methyl]thieno[3,2-d]pyrimidin-4-amine;
6-[(2S)-2-aminopropyl]-2-chloro-7-methyl-N-[(1,3-thiazol-2-yl)methyl]thieno[3,2-d]pyrimidin-4-amine;
6-[(2S)-2-aminopropyl]-2-chloro-7-methyl-N-[(3-methylfuran-2-yl)methyl]thieno[3,2-d]pyrimidin-4-amine;
6-[(2S)-2-aminopropyl]-2-chloro-7-methyl-N-[(5-methyl-1,3-thiazol-2-yl)methyl]thieno[3,2-d]pyrimidin-4-amine;
6-[(2S)-2-aminopropyl]-2-chloro-7-methyl-N-[(pyrazin-2-yl)methyl]thieno[3,2-d]pyrimidin-4-amine;
6-[(2S)-2-aminopropyl]-2-chloro-N-[(5-fluorothiophen-2-yl)methyl]-7-methylthieno[3,2-d]pyrimidin-4-amine
6-[(2S)-2-aminopropyl]-N-benzyl-2-chloro-7-methylthieno[3,2-d]pyrimidin-4-amine;
6-[(2S)-2-aminopropyl]-2-chloro-N-[(3-fluoropyridin-4-yl)methyl]-7-methylthieno[3,2-d]pyrimidin-4-amine;
6-[(2S)-2-aminopropyl]-2-chloro-7-cyclopropyl-N-[(furan-2-yl)methyl]thieno[3,2-d]pyrimidin-4-amine;
6-[(2S)-2-aminobutyl]-2-chloro-7-methyl-N-[(thiophen-2-yl)methyl]thieno[3,2-d]pyrimidin-4-amine;
6-[(2S)-2-aminopropyl]-2-bromo-N-[(furan-2-yl)methyl]-7-methylthieno[3,2-d]pyrimidin-4-amine;
6-[(2S)-2-aminopropyl]-2-chloro-7-methyl-N-[(1,2-oxazol-5-yl)methyl]thieno[3,2-d]pyrimidin-4-amine;
6-[(2S)-2-aminopropyl]-7-bromo-2-chloro-N-[(furan-2-yl)methyl]thieno[3,2-d]pyrimidin-4-amine;
6-(azetidin-3-yl)-2-chloro-N-[(furan-2-yl)methyl]-7-methylthieno[3,2-d]pyrimidin-4-amine;
7-bromo-2-chloro-N-[(furan-2-yl)methyl]thieno[3,2-d]pyrimidin-4-amine;
7-bromo-2-chloro-N-[(pyridin-4-yl)methyl]thieno[3,2-d]pyrimidin-4-amine;
6-[(2S)-2-aminopropyl]-2-chloro-N-[(2-fluorophenyl)methyl]-7-methylthieno[3,2-d]pyrimidin-4-amine;
6-[(2,S)-2-aminopropyl]-2-chloro-7-methyl-N-[(pyridin-4-yl)methyl]thieno[3,2-d]pyrimidin-4-amine;
6-[(1S)-1-aminoethyl]-7-bromo-2-chloro-N-[(furan-2-yl)methyl]thieno[3,2-d]pyrimidin-4-amine;
6-[(1S)-aminoethyl]-2-chloro-N-[(furan-2-yl)methyl]-7-methylthieno[3,2-d]pyrimidin-4-amine;
6-[(1S)-1-aminopropyl]-2-chloro-N-[(furan-2-yl)methyl]-7-methylthieno[3,2-d]pyrimidin-4-amine;
6-[(1R)-1-aminopropyl]-2-chloro-N-[(furan-2-yl)methyl]-7-methylthieno[3,2-d]pyrimidin-4-amine;
6-[(2S)-2-aminopropyl]-2-chloro-7-methyl-N-[(pyrimidin-4-yl)methyl]thieno[3,2 -d]pyrimidin-4-amine;
6-[(2S)-2-amino-4-fluorobutyl]-2-chloro-N-[(furan-2-yl)methyl]-7-methylthieno[3,2-d]pyrimidin-4-amine;
(4S)-4-[(2-chloro-4-{[(furan-2-yl)methyl]amino}-7-methylthieno[3,2-d]pyrimidin-6-yl)methyl]-1,3-oxazinan-2-one;
6-[(2S)-2-aminobutyl]-2-chloro-N-[(3-fluoropyridin-4-yl)methyl]-7-methylthieno[3,2-d]pyrimidin-4-amine;
6-[(2S)-2-aminobutyl]-2-chloro-7-methyl-N-[(1,3-thiazol-2-yl)methyl]thieno[3,2-d]pyrimidin-4-amine;
6-[(2S)-2-aminopropyl]-2-chloro-7-methyl-N-[(pyrimidin-2-yl)methyl]thieno[3,2-d]pyrimidin-4-amine;
(2R)-2′-amino-3-(2-chloro-7-methyl-4-{[(thiophen-2-yl)methyl]amino}thieno[3,2-d]pyrimidin-6-yl)propan-1-ol;
2-chloro-N-[(furan-2-yl)methyl]-7-methyl-6-(pyrrolidin-3-yl)thieno[3,2-d]pyrimidin-4-amine;
6-[(1S)-1-aminoethyl]-2-chloro-N-[(furan-2-yl)methyl]-7-phenylthieno[3,2-d]pyrimidin-4-amine;
6-[(2S)-2-aminopropyl]-2-chloro-N-[(3-fluoropyridin-2-yl)methyl]-7-methylthieno[3,2-d]pyrimidin-4-amine;
6-[(2S)-2-aminopropyl]-2-chloro-N-[2-fluoropyridin-3-yl)methyl]-7-methylthieno[3,2-d]pyrimidin-4-amine;
6-[(1S)-1-aminoethyl]-N-[(furan-2-yl)methyl]-2,7-diphenylthieno[3,2-d]pyrimidin-4-amine;
6-[(2S)-2-aminobutyl]-2-chloro-7-methyl-N-[(1,2-thiazol-5-yl)methyl]thieno[3,2-d]pyrimidin-4-amine;
3-(2-chloro-4-{[(furan-2-yl))methyl]amino}-7-methylthieno[3,2-d]pyrimidin-6-yl)propan-1-ol;
6-[(2S)-2-aminopropyl]-2-chloro-N-[(3,5-difluoropyridin-4-yl)methyl]-7-methylthieno[3,2-d]pyrimidin-4-amine;
6-[(2S)-2-aminopropyl]-2-chloro-N-[(furan-2-yl)methyl]-7-(4-methoxyphenyl)thieno[3,2-d]pyrimidin-4-amine;
(2S)-3-(2-chloro-4-{[(furan-2-yl)methyl]amino}-7-methylthieno[3,2-d]pyrimidin-6-yl)-2-methylpropan-1-ol;
6-(3-aminopropyl)-2-chloro-N-[(furan-2-yl)methyl]-7-methylthieno[3,2-d]pyrimidin-4-amine;
6-[(2S)-2-aminopropyl]-7-bromo-2-chloro-N-[(3-fluoropyridin-4-yl)methyl]thieno[3,2-d]pyrimidin-4-amine;
6-[(2S)-2-aminobutyl]-2-chloro-7-methyl-N-[(1,3-oxazol-2-yl)methyl]thieno[3,2-d]pyrimidin-4-amine;
6-[(2S)-3-amino-2-methylpropyl]-2-chloro-N-[(furan-2-yl)methyl]-7-methylthieno[3,2-d]pyrimidin-4-amine;
(2R)-3-(2-chloro-4-{[(furan-2-yl)methyl]amino}-7-methylthieno[3,2-d]pyrimidin-6-yl)-2-methylpropan-1-ol;
6-[(2R)-3-amino-2-methylpropyl]-2-chloro-N-[(furan-2-yl)methyl]-7-methylthieno[3,2-d]pyrimidin-4-amine;
6-[(2S)-2-aminopropyl]-2-chloro-N-[(1H-imidazol-2-yl)methyl]-7-methylthieno[3,2-d]pyrimidin-4-amine;
6-[(2S)-2-aminopropyl]-2-chloro-7-methyl-N-[(1,3-thiazol-5-yl)methyl]thieno[3,2-d]pyrimidin-4-amine;
6-[(2S)-2-aminopropyl]-2-chloro-7-methyl-N-[(1,3-oxazol-5-yl)methyl]thieno[3,2-d]pyrimidin-4-amine;
6-[(2R)-2-amino-3-methoxypropyl]-2-chloro-N-[(furan-2-yl)methyl]-7-methylthieno[3,2-d]pyrimidin-4-amine;
6-[(2S)-2-aminopropyl]-2-chloro-7-ethyl-N-[(furan-2-yl)methyl]thieno[3,2-d]pyrimidin-4-amine;
2-chloro-6-[(2S)-2-(cyclobutylamino)propyl]-N-[(furan-2-yl)methyl]-7-methylthieno[3,2-d]-pyrimidin-4-amine;
2-chloro-N-[(furan-2-yl)methyl]-7-methyl-6-[(2S)-2-(methylamino)propyl]thieno[3,2-d]pyrimidin-4-amine;
7-bromo-2-chloro-N-[(thiophen-2-yl)methyl]thieno[3,2-d]pyrimidin-4-amine;
6-[(2S)-2-aminopropyl]-2-chloro-7-methyl-N-[(1-methyl-1H-pyrazol-5-yl)methyl]thieno[3,2-d]pyrimidin-4-amine;
6-[(2S)-2-aminopropyl]-7-bromo-2-chloro-N-[(thiophen-2-yl)methyl]thieno[3,2-d]pyrimidin-4-amine;
6-[(2S)-2-aminopropyl]-7-bromo-4-{[(thiophen-2-yl)methyl]amino}thieno[3,2-d]pyrimidine-2-carbonitrile;
2-chloro-4-{[(thiophen-2-yl)methyl]amino}thieno[3,2-d]pyrimidine-7-carbonitrile;
6-[(2S)-2-aminopropyl]-4-{[(thiophen-2-yl)methyl]amino}thieno[3,2-d]pyrimidine-2,7-dicarbonitrile;
6-[(2S)-2-aminopropyl]-2-chloro-7-cyclopropyl-N-[(thiophen-2-yl)methyl]thieno[3,2-d]pyrimidin-4-amine;
6-[(2S)-2-aminopropyl]-2-chloro-7-phenyl-N-[(thiophen-2-yl)methyl]thieno[3,2-d]pyrimidin-4-amine;
6-[(2S)-2-aminopropyl]-2-chloro-7-(4-chlorophenyl)-N-[(thiophen-2-yl)methyl]thieno[3,2-d]pyrimidin-4-amine;
6-[(2S)-2-aminobutyl]-2-chloro-7-methyl-N-[(pyrimidin-4-yl)methyl]thieno[3,2-d]pyrimidin-4-amine;
6-[(2S)-2-aminopropyl]-2-chloro-N-[(3-fluorothiophen-2-yl)methyl]-7-methylthieno[3,2-d]pyrimidin-4-amine;
6-[(2S)-2-aminobutyl]-2-chloro-N-[(3-fluorothiophen-2-yl)methyl]-7-methylthieno[3,2-d]pyrimidin-4-amine;
6-[(2S)-2-aminopropyl]-2-chloro-N-[(4-fluoro-1,3-thiazol-2-yl)methyl]-7-methylthieno[3,2-d]pyrimidin-4-amine;
6-[(2S)-2-aminopropyl]-2-chloro-N-[(5-fluoro-1,3-thiazol-2-yl)methyl]-7-methylthieno[3,2-d]pyrimidin-4-amine;
6-[(1R)-1-aminoethyl]-2-chloro-N-[(furan-2-yl)methyl]-7-methylthieno[3,2-d]pyrimidin-4-amine;
2-chloro-N-[(pyrimidin-4-yl)methyl]thieno[3,2-d]pyrimidin-4-amine;
2-chloro-N-[(1,3-thiazol-2-yl)methyl]thieno[3,2-d]pyrimidin-4-amine;
(3S)-3-amino-4-(2-chloro-4-{[(furan-2-yl)methyl]amino}-7-methylthieno[3,2-d]pyrimidin-6-yl)butan-1-ol;
6-[(2S)-2-aminopropyl]-2-chloro-[(5-fluoropyrimidin-4-yl)methyl]-7-methylthieno[3,2-d]pyrimidin-4-amine, and
(2R)-2-amino-3-(2-chloro-7-methoxy-4-{[(thiophen-2-yl)methyl]amino}thieno[3,2-d]pyrimidin-6-yl)propan-1-ol;
wherein the form of the compound is selected from the group consisting of a salt, hydrate, solvate, and tautomer form thereof.

11. The method of claim 10, wherein the compound or form thereof is selected from the group consisting of:
6-[(2R)-2-amino-3-methylbutyl]-2-chloro-N-[(furan-2-yl)methyl]thieno[3,2-d]pyrimidin-4-amine;
6-[(2S)-2-aminopropyl]-2-chloro-N-[(furan-2-yl)methyl]-7-methylthieno[3,2-d]pyrimidin-4-amine;
6-[(2S)-2-aminopropyl]-N-[(furan-2-yl)methyl]-2,7-dimethylthieno[3,2-d]pyrimidin-4-amine;
6-[(2S)-2-aminopropyl]-2-chloro-7-methyl-N-[(5-methylfuran-2-yl)methyl]thieno[3,2-d]pyrimidin-4-amine;
6-[(2S)-2-aminopropyl]-2-chloro-7-methyl-N-[(thiophen-2-yl)methyl]thieno[3,2-d]pyrimidin-4-amine;
6-[(2S)-2-aminopropyl]-2-chloro-7-methyl-N-[(1,3-thiazol-2-yl)methyl]thieno[3,2-d]pyrimidin-4-amine;
6-[(2S)-2-aminopropyl]-2-chloro-7-methyl-N-[(5-methyl-1,3-thiazol-2-yl)methyl]thieno[3,2-d]pyrimidin-4-amine;
6-[(2S)-2-aminopropyl]-2-chloro-N-[(5-fluorothiophen-2-yl)methyl]-7-methylthieno[3,2-d]pyrimidin-4-amine;
6-[(2S)-2-aminopropyl]-2-bromo-N-[(furan-2-yl)methyl]-7-methylthieno[3,2-d]pyrimidin-4-amine;
6-[(2S)-2-aminopropyl]-2-chloro-7-methyl-N-[(1,2-oxazol-5-yl)methyl]thieno[3,2-d]pyrimidin-4-amine;
6-[(2S)-2-amino-4-fluorobutyl]-2-chloro-N-[(furan-2-yl))methyl]7-methylthieno[3,2-d]pyrimidin-4-amine;
(2R)-2-amino-3-(2-chloro-7-methyl-4-{[(thiophen-2-yl)methyl]amino}thieno[3,2-d]pyrimidin-6-yl)propan-1-ol;
6-[(2S)-2-aminopropyl]-2-chloro-N-[(3-fluoropyridin-2-yl)methyl]-7-methylthieno[3,2-d]pyrimidin-4-amine;
6-[(2S)-2-aminopropyl]-2-chloro-N-[(2-fluoropyridin-3-yl)methyl]-7-methylthieno[3,2-d]pyrimidin-4-amine;
6-[2S)-2-aminobutyl]-2-chloro-7-methyl-N-[(1,2-thiazol-5-yl)methyl]thieno[3,2-d]pyrimidin-4-amine;
6-[(2R)-3-amino-2-methylpropyl]-2-chloro-N-[(furan-2-yl)methyl]-7-methylthieno[3,2-d]pyrimidin-4-amine;
6-[(2S)-2-aminopropyl]-2-chloro-7-methyl-N-[(1,3-thiazol-5-yl)methyl]thieno[3,2-d]pyrimidin-4-amine;
6-[(2S)-2-aminopropyl]-2-chloro-7-methyl-N-[(1,3-oxazol-5-yl)methyl]thieno[3,2-d]pyrimidin-4-amine;
6-[(2S)-2-aminopropyl]-2-chloro-7-ethyl-N-[(furan-2-yl)methyl]thieno[3,2-d]pyrimidin-4-amine;
2-chloro-6-[(2S)-2-(cyclobutylamino)propyl]N-[(furan-2-yl)methyl]-7-methylthieno[3,2-d]pyrimidin-4-amine;
6-[(2S)-2-aminopropyl]-7-bromo-2-chloro-N-[(thiophen-2-yl)methyl]thieno[3,2-d]pyrimidin-4-amine;
6-[(2S)-2-aminopropyl]-2-chloro-7-cyclopropyl-N-[(thiophen-2-yl)methyl]thieno[3,2-d]pyrimidin-4-amine;
6-[(2S)-2-aminopropyl]-2-chloro-N-[(3-fluorothiophen-2-yl)methyl]-7-methylthieno[3,2-d]pyrimidin-4-amine;
6-[(2S)-2-aminopropyl]-2-chloro-N-[(4-fluoro-1,3-thiazol-2-yl)methyl]-7-methylthieno[3,2-d]pyrimidin-4-amine;
6-[(2′)-2-aminopropyl]-2-chloro-N-[(5-fluoro-1,3-thiazol-2-yl)methyl]-7-methylthieno[3,2-d]pyrimidin-4-amine; and
(3S)-3-amino-4-(2-chloro-4-{[(furan-2-yl)methyl]amino}-7-methylthieno[3,2-d]pyrimidin-6-yl)butan-1-ol;
wherein the form of the compound is selected from the group consisting of a salt, hydrate, solvate, and tautomer form thereof.

12. A method for treating a neurodegenerative disease characterized by the accumulation of aberrant forms of the microtubule associated protein Tau (MAPT) in a subject in need thereof, comprising administering to said subject an effective amount of a compound salt or form thereof selected form the group consisting of:
6-[(2S)-2-aminopropyl]-2-chloro-N-[(furan-2-yl)methyl]thieno[3,2-d]pyrimidin-4-amine dihydrochloride;
6-[(2S)-2-aminobutyl]-2-chloro-N-[(furan-2-yl)methyl]thieno[3,2-d]pyrimidin-4-amine dihydrochloride;
6-[(2R)-2-amino-3-methylbutyl]-2-chloro-N-[(furan-2-yl)methyl]thieno[3,2-d]pyrimidin-4-amine dihydrochloride;
6-[(2R,3S)-2-amino-3-methylpentyl]-2-chloro-N-[(furan-2-yl)methyl]thieno[3,2-d]pyrimidin-4-amine dihydrochloride;
6-[(2R)-2-amino-3,3-dimethylbutyl]-2-chloro-N-[(furan-2-yl)methyl]thieno[3,2-d]pyrimidin-4-amine dihydrochloride;
6-[(2S)-2-aminopropyl]-N-[(furan-2-yl)methyl]thieno[3,2-d]pyrimidin-4-amine dihydrochloride;
6-[(2S)-2-aminopropyl]-2-chloro-N-[(pyridin-4-yl)methyl]thieno[3,2-d]pyrimidin-4-amine dihydrochloride;
6-[(2S)-2-aminobutyl]-2-chloro-N-[(thiophen-2-yl)methyl]thieno[3,2-d]pyrimidin-4-amine dihydrochloride;
6-[(2S)-2-aminopropyl]-N-[(pyrimidin-4-yl)methyl]thieno[3,2-d]pyrimidin-4-amine dihydrochloride;
6-[(2S)-2-aminopropyl]-2-chloro-N-[(furan-2-yl)methyl]-7-methylthieno[3,2-d]pyrimidin-4-amine dihydrochloride;
6-[(2S)-2-aminobutyl]-2-chloro-N-[(furan-2-yl)methyl]-7-methylthieno[3,2-d]pyrimidin-4-amine dihydrochloride;
6-[(2S)-2-aminopropyl]-2-chloro-N-[(thiophen-2-yl)methyl]thieno[3,2-d]pyrimidin-4-amine dihydrochloride;
6-[(2S)-2-aminopropyl]-N-[(furan-2-yl)methyl]-2,7-dimethylthieno[3,2-d]pyrimidin-4-amine dihydrochloride;
6-[(2S)-2-aminopropyl]-2-ethyl-N-[(furan-2-yl)methyl]-7-methylthieno[3,2-d]pyrimidin-4-amine dihydrochloride;
6-[(2S)-2-aminopropyl]-2-cyclopropyl-N-[(furan-2-yl)methyl]-7-methylthieno[3,2-d]pyrimidin-4-amine dihydrochloride;
2-chloro-N-[(furan-2-yl)methyl]-7-methylthieno[3,2-d]pyrimidin-4-amine hydrochloride;
(2R)-2-amino-3-(2-chloro-4-{[(furan-2-yl)methyl]amino}-7-methylthieno[3,2-d]pyrimidin-6-yl)propan-1-ol dihydrochloride;
6-[(2S)-2-aminopropyl]-4-{[(furan-2-yl)methyl]amino}-7-methylthieno[3,2-d]pyrimidine-2-carboxamide trifluoroacetate;
6-[(2S)-2-aminopropyl]-4-{[(furan-2-yl)methyl]amino}-7-methylthieno[3,2-d]pyrimidine-2-carbonitrile trifluoroacetate;
6-[(2S)-2-aminopropyl]-2-chloro-7-methyl-N-[(5-methylfuran-2-yl)methyl]thieno[3,2-d]pyrimidin-4-amine dihydrochloride;
6-[(2S)-2-aminopropyl]-N-[(furan-2-yl)methyl]-7-methyl-2-(trifluoromethyl)thieno[3,2-d]pyrimidin-4-amine dihydrochloride;
6-[(2S)-2-aminopropyl]-2-chloro-7-methyl-N-[(4-methyl-1,3-thiazol-2-yl)methyl]thieno[3,2-d]pyrimidin-4-amine dihydrochloride;
6-[(2S)-2-aminopropyl]-2-chloro-7-methyl-N-[(thiophen-2-yl)methyl]thieno[3,2-d]pyrimidin-4-amine dihydrochloride;
6-[(2S)-2-aminopropyl]-2-chloro-7-methyl-N-[(1,3-thiazol-2-yl)methyl]thieno[3,2-d]pyrimidin-4-amine trifluoroacetate;
6-[(2S)-2-aminopropyl]-2-chloro-7-methyl-N-[(3-methylfuran-2-yl)methyl]thieno[3,2-d]pyrimidin-4-amine dihydrochloride;
6-[(2S)-2-aminopropyl]-2-chloro-7-methyl-N-[(5-methyl-1,3-thiazol-2-yl)methyl]thieno[3,2-d]pyrimidin-4-amine dihydrochloride;
6-[(2S)-2-aminopropyl]-2-chloro-7-methyl-N-[(pyrazin-2-yl)methyl]thieno[3,2-d]pyrimidin-4-amine dihydrochloride;
6-[(2S)-2-aminopropyl]-2-chloro-N-[(5-fluorothiophen-2-yl)methyl]-7-methylthieno[3,2-d]pyrimidin-4-amine dihydrochloride;
6-[(2S)-2-aminopropyl]-N-benzyl-2-chloro-7-methylthieno[3,2-d]pyrimidin-4-amine dihydrochloride;
6-[(2S)-2-aminopropyl]-2-chloro-N-[(3-fluoropyridin-4-yl)methyl]-7-methylthieno[3,2-d]pyrimidin-4-amine dihydrochloride;
6-[(2S)-2-aminopropyl]-2-chloro-7-cyclopropyl-N-[(furan-2-yl)methyl]thieno[3,2-d]pyrimidin-4-amine trifluoroacetate;
6-[(2′)-2-aminobutyl]-2-chloro-7-methyl-N-[(thiophen-2-yl)methyl]thieno[3,2-d]pyrimidin-4-amine dihydrochloride;
6-[(2S)-2-aminopropyl]-2-bromo-N-[(furan-2-yl)methyl]-7-methylthieno[3,2-d]pyrimidin-4-amine trifluoroacetate;
6-[(2S)-2-aminopropyl]-2-chloro-7-methyl-N-[(1,2-oxazol-5-yl)methyl]thieno[3,2-d]pyrimidin-4-amine hydrochloride;
6-[(2S)-2-aminopropyl]-7-bromo-2-chloro-N-[(furan-2-yl)methyl]thieno[3,2-d]pyrimidin-4-amine dihydrochloride;
6-(azetidin-3-yl)-2-chloro-N-[(furan-2-yl)methyl]-7-methylthieno[3,2-d]pyrimidin-4-amine trifluoroacetate;
6-[(2S)-2-aminopropyl]-2-chloro-N-[(2-fluorophenyl)methyl]-7-methylthieno[3,2-d]pyrimidin-4-amine dihydrochloride;
6-[(2S)-2-aminopropyl]-2-chloro-7-methyl-N-[(pyridin-4-yl)methyl]thieno[3,2-d]pyrimidin-4-amine dihydrochloride;
6-[(1S)-N-aminoethyl]-7-bromo-2-chloro-N-[(furan-2-yl)methyl]thieno[3,2-d]pyrimidin-4-amine hydrochloride;
6-[(1,S)-1-aminoethyl]-2-chloro-N-[(furan-2-yl)methyl]-7-methylthieno[3,2-d]pyrimidin-4-amine hydrochloride;
6-[(1S)-1-aminopropyl]-2-chloro-N-[(furan-2-yl)methyl]-7-methylthieno[3,2-d]pyrimidin-4-amine hydrochloride;
6-[(R)-1-aminopropyl]-2-chloro-N-[(furan-2-yl)methyl]-7-methylthieno[3,2-d]pyrimidin-4-amine hydrochloride;
6-[(2S)-2-aminopropyl]-2-chloro-7-methyl-N-[(pyrimidin-4-yl)methyl]thieno[3,2-d]pyrimidin-4-amine dihydrochloride;
6-[(2S)-2-amino-4-fluorobutyl]-2-chloro-N-[(furan-2-yl)methyl]-7-methylthieno[3,2-d]pyrimidin-4-amine dihydrochloride;
6-[(2S)-2-aminobutyl]-2-chloro-N-[(3-fluoropyridin-4-yl)methyl]-7-methylthieno[3,2-d]pyrimidin-4-amine dihydrochloride;
6-[(2S)-2-aminobutyl]-2-chloro-7-methyl-N-[(1,3-thiazol-2-yl)methyl]thieno[3,2-d]pyrimidin-4-amine dihydrochloride;
6-[(2S′)-2-aminopropyl]-2-chloro-7-methyl-N-[(pyrimidin-1-yl)methyl]thieno[3,2-d]pyrimidin-4-amine dihydrochloride;
(2R)-2-amino-3-(2-chloro-7-methyl-4-{[(thiophen-2-yl)methyl]amino}thieno[3,2-d]pyrimidin-6-yl)propan-1-ol dihydrochloride;
2-chloro-N-[(furan-2-yl)methyl]-7-methyl-6-(pyrrolidin-3-yl)thieno[3,2-d]pyrimidin-4-amine formate;
6-[(1S)-1-aminoethyl]-2-chloro-N-[(furan-2-yl)methyl]-7-phenylthieno[3,2-d]pyrimidin-4-amine formate;
6-[(2S)-2-aminopropyl]-2-chloro-N-[(3-fluoropyridin-2-yl)methyl]-7-methylthieno[3,2-d]pyrimidin-4-amine dihydrochloride;
6-[(2S)-2-aminopropyl]-2-chloro-N-[(2-fluoropyridin-3-yl)methyl]-7-methylthieno[3,2-d]pyrimidin-4-amine dihydrochloride;
6-[(1S)-N-aminoethyl]-N-[(furan-2-yl)methyl]-2,7-diphenylthieno[3,2-d]pyrimidin-4-amine formate;
6-[(2S)-2-aminobutyl]-2-chloro-7-methyl-N-[(1,2-thiazol-5-yl)methyl]thieno[3,2-d]pyrimidin-4-amine formate;
6-[(2S)-2-aminopropyl]-2-chloro-N-[(3,5-difluoropyridin-4-yl)methyl]-7-methylthieno[3,2-d]pyrimidin-4-amine dihydrochloride;
6-[(2S)-2-aminopropyl]-2-chloro-N-[(furan-2-yl)methyl]-7-(4-methoxyphenyl)thieno[3,2-d]pyrimidin-4-amine formate;
6-(3-aminopropyl)-2-chloro-N-[(furan-2-yl)methyl]-7-methylthieno[3,2-d]pyrimidin-4-amine hydrochloride;
6-[(2'S)-2-aminopropyl]-7-bromo-2-chloro-N-[(3-fluoropyridin-4-yl)methyl]thieno[3,2-d]pyrimidin-4-amine dihydrochloride;
6-[(2S)-2-aminobutyl]-2-chloro-7-methyl-N-[(1,3-oxazol-2-yl)methyl]thieno[3,2-d]pyrimidin-4-amine dihydrochloride;
6-[(2S)-3-amino-2-methylpropyl]-2-chloro-N-[(furan-2-yl)methyl]-7-methylthieno[3,2-d]pyrimidin-4-amine hydrochloride;
6-[(2R)-3-amino-2-methylpropyl]-2-chloro-N-[(furan-2-yl)methyl]-7-methylthieno[3,2-d]pyrimidin-4-amine hydrochloride;
6-[(2S)-2-aminopropyl]-2-chloro-N-[(1H-imidazol-2-yl)methyl]-7-methylthieno[3,2-d]pyrimidin-4-amine dihydrochloride;
6-[(2S)-2-aminopropyl]-2-chloro-7-methyl-N-[(1,3-thiazol-5-yl)methyl]thieno[3,2-d]pyrimidin-4-amine dihydrochloride;
6-[(2S)-2-aminopropyl]-2-chloro-7-methyl-N-[(1,3-oxazol-5-yl)methyl]thieno[3,2-d]pyrimidin-4-amine dihydrochloride;
6-[(2R)-2-amino-3-methoxypropyl]-2-chloro-N-[(furan-2-yl)methyl]-7-methylthieno[3,2-d]pyrimidin-4-amine dihydrochloride;
6-[(2S)-2-aminopropyl]-2-chloro-7-ethyl-N-[(furan-2-yl)methyl]thieno[3,2-d]pyrimidin-4-amine hydrochloride;
2-chloro-6-[(2S)-2-(cyclobutylamino)propyl]-N-[(furan-2-yl)methyl]-7-methylthieno[3,2-d]pyrimidin-4-amine formate;
2-chloro-N-[(furan-2-yl)methyl]-7-methyl-6-[(2S)-2-(methylamino)propyl]thieno[3,2-d]pyrimidin-4-amine hydrochloride;
6-[(2S)-2-aminopropyl]-2-chloro-7-methyl-N-[(1-methyl-11-pyrazol-5-yl)methyl]thieno[3,2-d]pyrimidin-4-amine hydrochloride;
6-[(2S)-2-aminopropyl]-7-bromo-2-chloro-N-[(thiophen-2-yl)methyl]thieno[3,2-d]pyrimidin-4-amine hydrochloride;
6-[(2S)-2-aminopropyl]-7-bromo-4-{[(thiophen-2-yl)methyl]amino}thieno[3,2-d]pyrimidine-2-carbonitrile formate;
6-[(2S)-2-aminopropyl]-4-{[(thiophen-2-yl)methyl]amino}thieno[3,2-d]pyrimidine-2,7-dicarbonitrile hydrochloride;
6-[(2S)-2-aminopropyl]-2-chloro-7-cyclopropyl-N-[(thiophen-2-yl)methyl]thieno[3,2-d]pyrimidin-4-amine formate;
6-[(2S)-2-aminopropyl]-2-chloro-7-phenyl-[(thiophen-2-yl)methyl]thieno[3,2-d]pyrimidin-4-amine hydrochloride;
6-[(2S)-2-aminopropyl]-2-chloro-7-(4-chlorophenyl)-N-[(thiophen-2-yl)methyl]thieno[3,2-d]pyrimidin-4-amine hydrochloride;
6-[(2S)-2-aminobutyl]-2-chloro-7-methyl-N-[(pyrimidin-4-yl)methyl]thieno[3,2-d]pyrimidin-4-amine dihydrochloride;
6-[(2S)-2-aminopropyl]-2-chloro-N-[(3-fluorothiophen-2-yl)methyl]-7-methylthieno[3,2-d]pyrimidin-4-amine dihydrochloride;
6-[(2S′)-2-aminobutyl]-2-chloro-N-[(3-fluorothiophen-2-yl)methyl]-7-methylthieno[3,2-d]pyrimidin-4-amine dihydrochloride;
6-[(2S)-2-aminopropyl]-2-chloro-N-[(4-fluoro-1,3-thiazol-2-yl)methyl]-7-methylthieno[3,2-d]pyrimidin-4-amine dihydrochloride;
6-[(2S)-2-aminopropyl]-2-chloro-N-[(5-fluoro-1,3-thiazol-2-yl)methyl]-methylthieno[3,2-d]pyrimidin-4-amine dihydrochloride;
6-[(1R)-1-aminoethyl]-2-chloro-N-[(furan-2-yl)methyl]-7-methylthieno[3,2-d]pyrimidin-4-amine hydrochloride;
(3S)-3-amino-4-(2-chloro-4-{[(furan-2-yl)methyl]amino}-7-methylthieno[3,2-d]pyrimidin-6-yl)butan-1-ol dihydrochloride;
6-[(2S)-2-aminopropyl]-2-chloro-N-[(5-fluoropyrimidin-4-yl)methyl]-7-methylthieno[3,2-d]pyrimidin-4-amine formate, and
(2R)-2-amino-3-(2-chloro-7-methoxy-4-{[(thiophen-2-yl)methyl]amino}thieno[3,2-d]pyrimidin-6-yl)propan-1-ol dihydrochloride;
wherein the form of the compound salt is selected from the group consisting of a salt, hydrate, solvate, and tautomer form thereof.

13. The method of claim 12, wherein the compound or form thereof is selected from the group consisting of:
6-[(2R)-2-amino-3-methylbutyl]-2-chloro-N-[(furan-2-yl)methyl]thieno[3,2-d]pyrimidin-4-amine dihydrochloride;
6-[(2S)-2-aminopropyl]-2-chloro-N-[(furan-2-yl)methyl]-7-methylthieno[3,2-d]pyrimidin-4-amine dihydrochloride;
6-[(2S)-2-aminopropyl]-N-[(furan-2-yl)methyl]-2,7-dimethylthieno[3,2-d]pyrimidin-4-amine dihydrochloride;
6-[(2S)-2-aminopropyl]-2-chloro-7-methyl-N-[(5-methylfuran-2-yl)methyl]thieno[3,2-d]pyrimidin-4-amine dihydrochloride;
6-[(2S)-2-aminopropyl]-2-chloro-7-methyl-N-[(thiophen-2-yl)methyl]thieno[3,2-d]pyrimidin-4-amine dihydrochloride;
6-[(2S)-2-aminopropyl]-2-chloro-7-methyl-N-[(1,3-thiazol-2-yl)methyl]thieno[3,2-d]pyrimidin-4-amine trifluoroacetate;
6-[(2S)-2-aminopropyl]-2-chloro-7-methyl-N-[(5-methyl-1,3-thiazol-2-yl)methyl]thieno[3,2-d]pyrimidin-4-amine dihydrochloride;
6-[(2S)-2-aminopropyl]-2-chloro-N-[(5-fluorothiophen-2-yl)methyl]-7-methylthieno[3,2-d]pyrimidin-4-amine dihydrochloride;
6-[(2S)-2-aminopropyl]-2-bromo-NV-[(furan-2-yl)methyl]-7-methylthieno[3,2-d]pyrimidin-4-amine trifluoroacetate;
6-[(2S)-2-aminopropyl]-2-chloro-7-methyl-N-[(1,2-oxazol-5-yl)methyl]thieno[3,2-d]pyrimidin-4-amine hydrochloride;
6-[(2S)-2-amino-4-fluorobutyl]-2-chloro-N-[(furan-2-yl)methyl]-7-methylthieno[3,2-d]pyrimidin-4-amine dihydrochloride;
(2R)-2-amino-3-(2-chloro-7-methyl-4-{[(thiophen-2-yl)methyl]amino}thieno[3,2-d]pyrimidin-6-yl)propan-1-ol dihydrochloride;
6-[(2S)-2-aminopropyl]-2-chloro-N-[(3-fluoropyridin-2-yl)methyl]-7-methylthieno[3,2-d]pyrimidin-4-amine dihydrochloride;
6-[(2S)-2-aminopropyl]-2-chloro-N-[2-fluoropyridin-3-yl)methyl]-7-methylthieno[3,2-d]pyrimidin-4-amine dihydrochloride;
6-[(S′)-2-aminobutyl]-2-chloro-7-methyl-N-[(1,2-thiazol-5-yl)methyl]thieno[3,2-d]pyrimidin-4-amine formate;
6-[(2S)-2-aminopropyl]-2-chloro-N-[(3,5-difluoropyridin-4-yl)methyl]-7-methylthieno[3,2-d]pyrimidin-4-amine dihydrochloride;
6-[(2R)-3-amino-2-methylpropyl]-2-chloro-N-[(furan-2-yl)methyl]-7-methylthieno[3,2-d]pyrimidin-4-amine hydrochloride;
6-[(2S)-2-aminopropyl]-2-chloro-7-methyl-N-[(1,3-thiazol-5-yl)methyl]thieno[3,2-d]pyrimidin-4-amine dihydrochloride;
6-[(2S)-2-aminopropyl]-2-chloro-7-methyl-N-[(1,3-oxazol-5-yl)methyl]thieno[3,2-d]pyrimidin-4-amine dihydrochloride;
6-[(2S)-2-aminopropyl]-2-chloro-7-ethyl-7-[(furan-2-yl)methyl]thieno[3,2-d]pyrimidin-4-amine hydrochloride;
2-chloro-6-[(2S)-2-(cyclobutylamino)propyl]-N-[(furan-2-yl)methyl]-7-methylthieno[3,2-d]pyrimidin-4-amine formate;
6-[(2S)-2-aminopropyl]-7-bromo-2-chloro-N-[(thiophen-2-yl)methyl]thieno[3,2-d]pyrimidin-4-amine hydrochloride;
6-[(2S)-2-aminopropyl]-2-chloro-7-cyclopropyl-N-[(thiophen-2-yl)methyl]thieno[3,2-d]pyrimidin-4-amine formate;
6-[(2S)-2-aminopropyl]-2-chloro-N-[(3-fluorothiophen-2-yl)methyl]-7-methylthieno[3,2-d]pyrimidin-4-amine dihydrochloride;
6-[(2S)-2-aminopropyl]-2-chloro-N-[(4-fluoro-1,3-thiazol-2-yl)methyl]-7-methylthieno[3,2-d]pyrimidin-4-amine dihydrochloride;
6-[(2S)-2-aminopropyl]-2-chloro-N-[(5-fluoro-1,3-thiazol-2-yl)methyl]-7-methylthieno[3,2-d]pyrimidin-4-amine dihydrochloride; and
(3)-3-amino-4-(2-chloro-4-{[(furan-2-yl)methyl]amino}-7-methylthieno[3,2-d]pyrimidin-6-yl)butan-1-ol dihydrochloride;
wherein the form of the compound salt is selected from the group consisting of a hydrate, solvate, and tautomer form thereof.

14. The method of claim 1, wherein the effective amount of the compound or form thereof induces exon 10 skipping in MAPT mRNA in the subject.

15. The method of claim 1, wherein the effective amount of the compound or form thereof lowers MAPT4R protein in the subject.

16. The method of claim 1, wherein the neurogenerative disease is selected from the group consisting of Alzheimer's disease, dementia pugilistica, Guam Amyotrophic lateral sclerosis-Parkinsonism-Dementia (Guam ALS/PD), Pick Disease, Argyrophilic grain dementia, Nieman-Pick type C, Subacute sclerosing panencephalitis (SSPE), Progressive supranuclear palsy (PSP), multisystem atrophy (MSA), Corticobasoganlionic degeneration, Frontotemporal dementia with parkinsonism-17 (FTDP-17), Postencephalitic Parkinsonism (PEP), Autosomal recessive Parkinsonism, frontotemporal dementia, and progressive supranuclear palsy.

17. The method of claim 1, wherein the effective amount of the compound or form thereof is in an admixture with one or more pharmaceutically acceptable excipient(s).


SEQUENCE LISTING (6 sequences)

SEQ ID NO: 1 - DNA, 21 bp, Homo sapiens
  source 1..21: mol_type = other DNA
caacggatttggtcgtattgg

SEQ ID NO: 2 - DNA, 25 bp, Homo sapiens
  source 1..25: mol_type = other DNA
tgatggcaacaatatccactttacc

SEQ ID NO: 3 - DNA, 21 bp, Homo sapiens
  source 1..21: mol_type = other DNA
cgcctggtcaccagggctgct

SEQ ID NO: 4 - DNA, 19 bp, Homo sapiens
  source 1..19: mol_type = other DNA
aggcgggaaggtgcaaata

SEQ ID NO: 5 - DNA, 23 bp, Homo sapiens
  source 1..23: mol_type = other DNA
ctggtttatgatggatgttgcct

SEQ ID NO: 6 - DNA, 32 bp, Homo sapiens
  source 1..32: mol_type = other DNA
tctacaaaccagttgacctgagcaaggtgacc